(19) United States
(12) Patent Application Publication
(10) Pub. No.: US 2020/0061167 A1
(43) Pub. Date: Feb. 27, 2020

(54) COMBINATION OF LISTERIA-BASED VACCINE WITH ANTI-CTLA-4 OR ANTI-CD137 ANTIBODIES

(71) Applicant: ADVAXIS, INC., Princeton, NJ (US)

(72) Inventors: Sandra M. HAYES, Hightstown, NJ (US); Rachelle KOSOFF, Newtown, PA (US); Jun ZOU, Cranbury, NJ (US)

(73) Assignee: ADVAXIS, INC., Princeton, NJ (US)

(21) Appl. No.: 16/346,855

(22) PCT Filed: Nov. 7, 2017
(86) PCT No.: PCT/US2017/060444

§ 371 Date: May 1, 2019

Related U.S. Application Data
(60) Provisional application No. 62/418,690, filed on Nov. 7, 2016.

(51) Int. Cl. A61K 39/00; C07K 16/28; A61K 39/395; A61K 39/12; A61P 35/00

(52) U.S. Cl. CPC A61K 39/0011; C07K 16/2878; A61K 39/3955; A61K 39/12; A61K 2039/585; A61K 2039/545; A61K 2039/523; C12N 2710/20034; A61P 35/00

(57) ABSTRACT

The subject matter described herein is directed to methods of treating, protecting against, and inducing an immune response against a human papillomavirus-associated tumor or cancer, comprising the step of administering to a subject a recombinant Listeria strain expressing a construct comprising at least one human papillomavirus antigen in combination with one or more other therapeutic agents to treat a tumor or metastatic cancer.

### FIELD OF INVENTION

The present invention provides methods of treating, protecting against, and inducing an immune response against a human papillomavirus-associated tumor or cancer, comprising the step of administering to a subject a recombinant Listeria strain expressing a construct comprising at least one human papillomavirus antigen in combination with one or more other therapeutic agents to treat a tumor or metastatic cancer.

### BACKGROUND OF THE INVENTION

Human papillomaviruses (HPVs) are the causes of many cancers, including cervical, anal, vulvar, vaginal, penile and oropharyngeal (Zhang G L, et al., “HPVdb: a data mining system for knowledge discovery in human papillomavirus with applications in T cell immunology & vaccinology”. Database, 2014, 1-12). The prevalence of human papillomavirus (HPV)-associated oropharyngeal cancer (HPVOPC) is increasing in the USA (225% from 1988 to 2004). HPVOPC patients tend to be younger and have a favorable prognosis, with a 69% reduction in the risk of death compared with HPV-negative patients. However most HPVOPC patients present with advanced stage, and standard chemoradiation regimens can be associated with significant toxicity. Thus the patients who have a good prognosis are paradoxically at greater risk of therapy-related long-term poor quality-of-life outcomes. Immunotherapy has the potential to reduce toxicity through de-escalation of chemoradiation regimens, and potentially enhance long-term disease control.

The HR-HPV E6 and E7 proteins are consistently expressed in dysplasias and carcinomas, disrupting the cell cycle regulatory proteins p53 and pRb, respectively. The obligatory expression of E6 and E7 by both dysplastic and invasive malignant lesions, as well as the viral origin of these proteins, make them excellent targets for HPV therapeutic vaccines.

Listeria monocytogenes (Lm) is a food-borne Gram-positive bacterium that can occasionally cause disease in humans, in particular the elderly, newborns, pregnant women and immunocompromised individuals. In addition to strongly activating innate immunity and inducing a cytokine response that enhances antigen-presenting cell (APC) function, Lm has the ability to replicate in the cytosol of APCs after escaping from the phagosome, mainly through the action of the listeriolysin O (LLO) protein. This unique intracellular life cycle allows antigens secreted by Lm to be processed and presented in the context of both MHC class I and II molecules, resulting in potent cytotoxic CD8+ and Th1 CD4⁺ T-cell-mediated immune responses.

The present invention addresses the need for improved treatment modalities in patients having human papillomavirus (HPV)-associated cancer by providing a Listeria monocytogenes-based immunotherapy comprising at least one HPV antigen in combination with one or more other therapeutic agents to treat a tumor or metastatic cancer and use of the same for preventing and treating HPV-related cancers.

### SUMMARY OF THE INVENTION

In one aspect, the present invention relates to a method of promoting an antigen-specific memory T cell population comprising, administering to a subject an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CTLA-4 antibody, an anti-CD137 antibody. or a functional fragment thereof.

In another aspect, the present invention relates to a method for preventing reoccurrence of a tumor in a subject in need thereof. the method comprising administering to the subject an effective amount of a combination comprising, i. an immunogenic composition comprising a recombinant Listeria strain comprising a nucleic acid molecule, the nucleic acid molecule comprising a first open reading frame encoding a fusion polypeptide. wherein the fusion polypeptide comprises a truncated listeriolysin O (tLLO) protein, a truncated ActA protein, or a PEST amino acid sequence fused to a heterologous antigen or fragment thereof. and ii. an effective amount of a composition comprising an anti-CTLA-4 antibody, an anti-CD137 antibody, or a functional fragment thereof.

In another aspect, the present invention relates to a method for treating metastatic cancer in a subject in need thereof, the method comprising administering to the subject an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CTLA-4 antibody, an anti-CD137 antibody, or a functional fragment thereof.

In another aspect. the present invention relates to a method for preventing metastasis in a cancer patient at risk for metastasis, the method comprising administering to the patient an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CTLA-4 antibody, an anti-CD137 antibody, or a functional fragment thereof.

In another aspect, the present invention relates to the methods described above wherein a first dose of the composition comprising a CTLA-4 antibody, an anti-CD137 antibody, or a functional fragment thereof is administered about 24-72 hours after the administration of a first dose of the immunogenic composition comprising a recombinant Listeria strain.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1 shows Lm-E7 and Lm-LLO-E7 use different expression systems to express and secrete E7. Lm-E7 was generated by introducing a gene cassette into the orfZ domain of the L. monocytogenes genome (A). The hly promoter drives expression of the hly signal sequence and the first five amino acids (AA) of LLO followed by HPV-16 E7. B), Lm-LLO-E7 was generated by transforming the prfA-strain XFL-7 with the plasmid pGG-55. pGG-55 has the hly promoter driving expression of a nonhemolytic fusion of LLO-E7. pGG-55 also contains the prfA gene to select for retention of the plasmid by XFL-7 in vivo.

FIG. 2 shows Lm-E7 and Lm-LLO-E7 secrete E7. Lm-Gag (lane 1), Lm-E7 (lane 2), Lm-LLO-NP (lane 3), Lm-LLO-E7 (lane 4), XFL-7 (lane 5), and 10403S (lane 6) were grown overnight at 37° C. in Luria-Bertoni broth. Equivalent numbers of bacteria, as determined by OD at 600 nm absorbance, were pelleted and 18 ml of each supernatant was TCA precipitated. E7 expression was analyzed by Western blot. The blot was probed with an anti-E7 mAb, followed by HRP-conjugated anti-mouse (Amersham), then developed using ECL detection reagents.

FIG. 3 shows tumor immunotherapeutic efficacy of LLO-E7 fusions. Tumor size in millimeters in mice is shown at 7, 14, 21, 28 and 56 days post tumor-inoculation. Naive mice: open-circles; Lm-LLO-E7: filled circles; Lm-E7: squares; Lm-Gag: open diamonds; and Lm-LLO-NP: filled triangles.

FIG. 4 shows splenocytes from Lm-LLO-E7-immunized mice proliferate when exposed to TC-1 cells. C57BL/6 mice were immunized and boosted with Lm-LLO-E7, Lm-E7, or control rLm strains. Splenocytes were harvested 6 days after the boost and plated with irradiated TC-1 cells at the ratios shown. The cells were pulsed with ³H thymidine and harvested. Cpm is defined as (experimental cpm)—(no-TC-1 control).

FIG. 5 shows (A) induction of E7-specific IFN-gamma-secreting CD8⁺ T cells in the spleens and the numbers penetrating the tumors, in mice administered TC-1 tumor cells and subsequently administered Lm-E7, Lm-LLO-E7, Lm-ActA-E7, or no vaccine (naive) and (B) induction and penetration of E7 specific CD8⁺ cells in the spleens and tumors of the mice described for (A).

FIG. 6 shows Listeria constructs containing PEST regions induce a higher percentage of E7-specific lymphocytes within the tumor. A. representative data from 1 experiment. B. average and SE of data from all 3 experiments.

FIG. 7A shows effect of passaging on bacterial load (virulence) of recombinant Listeria vaccine vectors. Top panel. Lm-Gag. Bottom panel. Lm-LLO-E7. FIG. 7B shows effect of passaging on bacterial load of recombinant Lm-E7 in the spleen. Average CFU of live bacteria per milliliter of spleen homogenate from four mice is depicted.

FIG. 8 shows induction of antigen-specific CD8⁺ T-cells for HIV-Gag and LLO after administration of passaged Lm-Gag versus unpassaged Lm-Gag. Mice were immunized with 10³ (A, B, E, F) or 10³ (C, D, G, H) CFU passaged Listeria vaccine vectors, and antigen-specific T-cells were analyzed. B, D, F, H: unpassaged Listeria vaccine vectors. A-D immune response to MHC class I HIV-Gag peptide. E-H: immune response to an LLO peptide. I: splenocytes from mice immunized with 10³ CFU passaged Lm-Gag stimulated with a control peptide from HPV E7.

FIG. 9A shows plasmid isolation throughout LB stability study. FIG. 9B shows plasmid isolation throughout TB stability study. FIG. 9C shows quantitation of TB stability study.

FIG. 10 shows numbers of viable bacteria chloramphenicol (CAP)-resistant and CAP-sensitive colony-forming units (CFU) from bacteria grown in LB. Dark bars: CAP⁺; white bars: CAP⁻. The two dark bars and two white bars for each time point represent duplicate samples.

FIG. 11 shows numbers of viable bacteria CAP-resistant and CAP-sensitive CFU from bacteria grown in TB. Dark bars: CAP⁺; white bars: CAP. The two dark bars and two white bars for each time point represent duplicate samples.

FIG. 12 shows actual chromatograms showing the region of the D133V mutation (arrows). The mixture ratio is shown in parentheses.

FIG. 13 shows representation of the location of the ADV451, 452 and 453 primers and the segment of the prfA gene amplified in the reaction.

FIG. 14 shows specificity of the PCR reaction using primers ADV451 and ADV453.

FIG. 15 shows specificity of the PCR reaction using primers ADV452 and ADV453.

FIG. 16 shows sensitivity of the PCR reaction to detect the wild-type prfA sequence using the primer ADV452 and 1 ng as the initial amount of DNA.

FIG. 17 shows sensitivity of the PCR reaction to detect the wild-type prfA sequence using the primer ADV452 and 5 ng as the initial amount of DNA.

FIG. 18 shows average density of the bands from the PCR depicted in FIG. 16.

FIG. 19 shows average density of the bands from the PCR depicted in FIG. 17.

FIG. 20 shows validation of the PCR reaction to detect the wild-type prfA sequence using the primer ADV452.

FIG. 21 shows average density of the bands from the PCR depicted in FIG. 16.

FIG. 22 shows analysis of the D133V PrfA mutation in the Lm-LLO-E7: (A) Original image used for densitometry; (B) Image was digitally enhanced to facilitate the visualization of the low density bands.

FIG. 23 shows (A) schematic representation of the chromosomal region of the Lmdd-143 and LmddA-143 after k/k3 integration and actA deletion and (B) the k/k3 gene is integrated into the Lmdd and LmddA chromosome. PCR from chromosomal DNA preparation from each construct using k/k3 specific primers amplifies a band of 714 bp corresponding to the k/k3 gene, lacking the secretion signal sequence of the wild type protein.

FIG. 24 shows (A) map of the pADV134 plasmid, (B) proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3), (C) Map of the pADV142 plasmid, and (D) western blot showed the expression of LLO-PSA fusion protein using anti-PSA and anti-LLO antibody.

FIG. 25 shows LmHPV dose titration for selection of subtherapeutic dose. Subtherapeutic dose 3×10⁷ selected for combination studies.

FIG. 26 shows dosing schedule for combination therapy (LM-HPV plus mAb).

FIG. 27A shows combining a subtherapeutic dose of AXAL with either anti-CD137 mAb or anti-CTLA4 mAb significantly enhanced tumor control. FIG. 27B shows combining a subtherapeutic dose of AXAL with either anti-CD137 mAb or anti-CTLA4 mAb significantly prolonged animal survival.

FIG. 28 shows the anti-tumor effects of the AXAL+anti-CD137 mAb or the AXAL+anti-CTLA-4 mAb combination required HPV-E7 protein expression.

FIG. 29 shows dosing schedule for optimization.

FIG. 30 shows results for tumor volume measured in varying doses of anti-CD137, anti-CTLA-4, and anti-TIM-3 antibody combination therapy with LmHPV.

FIG. 31A shows results for tumor volume measure in varying doses of anti-LAG3, anti-TIM-3 and anti-CTLA-4 antibody combination therapy with LmHPV. FIG. 31B shows results for tumor volume measure in varying doses of GITR and CD 137 antibody combination therapy with LmHPV.

FIG. 32 shows survival at Day 81 combination therapy with varying doses of mAb.

FIG. 33 shows results for tumor re-challenge study with LmHPV and CD137, TIM-3, or CTLA-4 combination therapy.

FIG. 34 shows tumor growth inhibition after mono- or combo-treatment with LmHPV and anti-CD137 Ab.

FIGS. 35A and 35B shows results for tumor volume measure in triple combination study.

FIG. 36 shows survival at Day 52 for triple combination study.

FIG. 37 shows tumor volume results show anti-tumor efficacy of combined treatment with LmHPV+αCD137 is CD4 and CD8 cell dependent.

FIGS. 38A and 38B shows immune phenotyping of tumor. FIG. 38A shows results for CD45+, CD8+, Treg, CD8/Treg cells. FIG. 38B shows HPV-E7 specific CD8+ T cells, E7+CD44+CD62L+, CD103+Treg, KLRG1+ results.

FIG. 39 shows survival at Day 45 combination therapy with varying doses of mAb.

FIG. 40 shows survival at Day 45 days post-tumor implantation.

FIG. 41 shows median survival of AXAL with various antibody-based immunotherapies.

FIG. 42 shows cellular changes in the tumor microenvironment as a result of combination therapy.

FIG. 43 shows increased percentages of effector cell subsets and mature dendritic cells were observed in the tumor after combination therapy.

FIG. 44 shows decreased percentages of suppressor cell subsets were observed in the tumor after combination therapy.

FIG. 45 shows CD8+ HPV-E7+ in blood of rechallenged mice.

FIG. 46 shows CD8+ HPV-E7+ Effectors (CD44+) in rechallenged mice.

FIG. 47 shows HPV-E7 measured at baseline prior to tumor rechallenge. 47 days post primary tumor implantation. Animals tumor free for at least 24 days prior to rechallenge.

FIG. 48 shows HPV-E7+CD8 T cells 3 weeks after rechallenge.

FIG. 49 shows combo therapy administration schedule for Examples 16 and 18.

FIG. 50 shows (A) experimental procedure and (B) results for kinetics of CD137 expression on T cells following AXAL treatment.

FIG. 51 shows tumor growth curves.

FIG. 52 shows animal survival at day 65.

FIG. 53 shows tumor regression induced by AXAL and anti-CD137 mAb is associated with increased levels of tumor-infiltrating HPV-E7-specific CD8+ T cells.

FIG. 54 shows tumor growth curves (AXAL±anti-CD137 mAb±anti-CTLA-4 mAb).

FIG. 55 shows animal survival at day 47 (AXAL±anti-CD137 mAb±anti-CTLA-4 mAb).

FIG. 56 shows tumor growth curves (AXAL±anti-CD137 mAb±anti-PD-1 mAb).

FIG. 57 shows animal survival at day 47 (AXAL±anti-CD137 mAb±anti-PD-1 mAb).

FIG. 58 shows combo therapy administration schedule for Example 19.

FIG. 59 shows tumor growth curves.

FIG. 60 shows study design for ADXS-PSA monotherapy arm of the KEYNOTE-046 trial.

FIG. 61 shows key baseline demographics of study participants in Part A.

FIG. 62 shows (A) dosing and blood draw schedules in ADXS-PSA monotherapy phase and (B) ADXS-PSA monotherapy upregulates expression of TNFRSF9, the gene encoding CD137, in stable disease and non-stable disease metastatic castration-resistant prostate cancer (mCRPC) patients.

FIG. 63 shows only stable disease patients upregulate expression of PDCD1, the gene encoding PD-1, following ADXS-PSA treatment.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The disclosure relates to compositions and methods for treating a cancer. Specifically, the disclosure relates to administering a Listeria-based immunogenic composition in combination with one or more other therapeutic agents to treat a cancer.

Examples of therapeutic agents include, for example, but not limited to, an anti-CTLA4 antibody or anti-4-1BB (CD 137) antibody. or a combination thereof.

In one embodiment, disclosed is a method for inducing an anti-tumor or anti-cancer immune response in a subject having a tumor or cancer, the method comprising the step of administering an effective amount of a combination therapy comprising a recombinant Listeria strain, at least one therapeutic agent for treating cancer, thereby inducing an anti-tumor or anti-cancer immune response in said subject.

In another embodiment, a therapeutic agent comprises an anti-CTLA4 antibody or anti-4-1BB (CD 137) antibody, or a fragment or combination thereof.

In another embodiment, a disease disclosed herein is a tumor or tumor growth, or a cancer.

In another embodiment, a disease disclosed herein is a human papillomavirus (HPV)-associated cancer.

In another embodiment, a disease disclosed herein is a metastatic cancer.

Listeria-Based Immunogenic Compositions

It will be appreciated by a skilled artisan that a Listeria-based immunogenic composition may include, for example, a recombinant Listeria strain.

In one embodiment, a composition comprising a recombinant Listeria strain expressing a recombinant polypeptide comprising a listeriolysin O (LLO) fragment and at least one antigen or a fragment associated with a disease thereof and methods of treating, protecting against, and inducing an immune response against a disease, comprising the step of administering the composition comprising the recombinant Listeria strain.

In one embodiment, the nucleic acid molecule disclosed herein comprises a first open reading frame encoding recombinant polypeptide comprising a heterologous antigen or fragment thereof. In another embodiment, the recombinant polypeptide further comprises an N-terminal LLO fused to the heterologous antigen. In another embodiment, the nucleic acid molecule disclosed herein further comprises a second open reading frame encoding a metabolic enzyme. In another embodiment, the metabolic enzyme complements an endogenous gene that is lacking in the chromosome of the recombinant Listeria strain.

In another embodiment, a recombinant Listeria strain disclosed herein comprises a recombinant nucleic acid construct comprising a first open reading frame encoding a recombinant polypeptide comprising an N-terminal fragment of an LLO protein operably linked to or fused to at least one heterologous antigen or fragment thereof. In another embodiment, the recombinant Listeria strain comprises a recombinant nucleic acid construct comprising a first open reading frame encoding a recombinant polypeptide comprising an N-terminal fragment of an LLO protein operably linked or fused to more than one heterologous antigen or fragment thereof.

The N-terminal LLO protein fragment and heterologous antigen are, in another embodiment, fused directly to one another. In another embodiment, the genes encoding the N-terminal LLO protein fragment and the heterologous antigen are fused directly to one another. In another embodiment, the N-terminal LLO protein fragment and the heterologous antigen are attached via a linker peptide. In another embodiment, the N-terminal LLO protein fragment and the heterologous antigen are attached via a heterologous peptide. In another embodiment, the N-terminal LLO protein fragment is N-terminal to the heterologous antigen. In another embodiment, the N-terminal LLO protein fragment is the N-terminal-most portion of the fusion protein.

In one embodiment, the present invention provides a method of inducing an anti-tumor or an anti-cancer immune response in a human subject, the method comprising the step of administering to said subject a composition comprising a recombinant Listeria strain comprising a recombinant nucleic acid, said nucleic acid comprising a first open reading frame encoding a recombinant polypeptide comprising an N-terminal fragment of an LLO protein fused to a heterologous antigen or fragment thereof, thereby inducing an immune response against a tumor or a cancer expressing said heterologous antigen or fragment thereof. In another embodiment, a subtherapeutic dose of a recombinant Listeria strain in combination with a subtherapeutic dose of a therapeutic agent reduces reduces the severity of side effects while achieving an effective anti-tumor response. In another embodiment, a therapeutic agent comprises an anti-CTLA-4 antibody or anti-4-1BB (CD137) antibody. or a fragment or combination thereof.

It will be appreciated by a skilled artisan that the composition comprising a recombinant Listeria provided herein may be administered in combination with other treatment modalities, including, but not limited to, chemotherapy, radiation, therapeutic agent such as anti-CTLA-4 antibody or anti-4-1BB (CD137) antibody, or a fragment or combination thereof. In another embodiment, administration of a recombinant Listeria and a subtherapeutic dose of anti-CTLA-4 antibody or anti-CD137 antibody achieves an effective anti-tumor response while the associated toxicity of the anti-CTLA-4 antibody or anti-CD137 is reduced.

In another embodiment, administration of the Listeria disclosed herein or the Listeria-based immunotherapy disclosed herein is able to reduce the need of a subject having a tumor or a cancer to receive chemotherapeutic or radiation treatment. In another embodiment, administration of the Listeria disclosed herein or the Listeria-based immunotherapy disclosed herein is able to eliminate the need for a subject having a tumor or cancer to receive radiation or chemotherapy. In another embodiment, administration of the Listeria disclosed herein or the Listeria-based immunotherapy disclosed herein is able to reduce the severity of side effects associated with a radiation or chemotherapy treatment in a subject having a tumor or cancer. In another embodiment, administration of the Listeria disclosed herein or the Listeria-based immunotherapy disclosed herein allows for the administration of a subtherapeutic dose of a therapeutic agent such as anti-CTLA-4 antibody or anti-4-1BB (CD137) antibody, or a fragment or combination thereof while achieving an effective anti-tumor response.

In one embodiment, the present invention also provides methods for inducing an anti-tumor antigen-specific cytotoxic T-cell (CTL) response in a human subject and treating disorders, and symptoms associated with said disease comprising administration of the recombinant Listeria strain.

In one embodiment, disclosed herein is a recombinant Listeria strain, said recombinant Listeria strain comprising a recombinant nucleic acid, said nucleic acid comprising a first open reading frame encoding a recombinant polypeptide comprising a first an N-terminal fragment of an LLO protein fused to a heterologous antigen or fragment thereof, and wherein said recombinant nucleic acid further comprises a second open reading frame encoding a mutant prfA gene. In one embodiment, the mutant prfA gene is one that encodes a point mutation from amino acid D (which also known as “Asp,” “Aspartate” or “Aspartic acid”) to amino acid V (which is also known as “Val,” or “Valine”) at amino acid position 133. In one embodiment, a recombinant Listeria strain disclosed herein comprises a mutation or deletion in the endogenous prfA gene. In another embodiment, a chromosomal mutation or deletion in a prfA gene in a Listeria disclosed herein is complemented via a plasmid comprising a nucleic acid sequence encoding a mutant prfA gene encoding a mutant PrfA protein comprising a D133V amino acid substitution. In another embodiment, a mutant PrfA protein comprising a D133V amino acid substitution complements an endogenous prfA mutation in a Listeria disclosed herein.

In another embodiment, the recombinant Listeria is an attenuated Listeria. It will be appreciated that the terms “attenuation” or “attenuated” may encompass a bacterium, virus, parasite, infectious organism, prion, tumor cell, gene in the infectious organism, and the like, that is modified to reduce toxicity to a host. The host can be a human or animal, or an organ, tissue, or cell. The bacterium, to give a non-limiting example, can be attenuated to reduce binding to a host cell, to reduce spread from one host cell to another host cell, to reduce extracellular growth, or to reduce intracellular growth in a host cell. In one embodiment, attenuation can be assessed by measuring, e.g., an indicum or indicia of toxicity, the LD₅₀, the rate of clearance from an organ, or the competitive index (see, e.g., Auerbuch, et al. (2001) Infect. Immunity 69:5953-5957). Generally, an attenuation results in an increase in the LD₅₀ and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and most often by at least 100,000-fold. In another embodiment, attenuation results in an increase in the LD₅₀ and/or an increase in the rate of clearance by at least 25%. In another embodiment, attenuation results in an increase in the LD₅₀ and/or an increase in the rate of clearance by 3-5 fold. In other embodiments, attenuation results in an increase in the LD₅₀ and/or an increase in the rate of clearance by 5-10 fold, 11-20 fold, 21-30 fold, 31-40 fold, 41-50 fold, 51-100 fold, 101-500 fold, 501-1,000 fold, 1001-10,000 fold, or 10,001-100,000 fold.

It will be well appreciated by a skilled artisan that the term “Attenuated gene” may encompass a gene that mediates toxicity, pathology, or virulence, to a host, growth within the host, or survival within the host, where the gene is mutated in a way that mitigates, reduces, or eliminates the toxicity, pathology, or virulence. The reduction or elimination can be assessed by comparing the virulence or toxicity mediated by the mutated gene with that mediated by the non-mutated (or parent) gene. “Mutated gene” encompasses deletions, point mutations, inversions, truncations, and frameshift mutations in regulatory regions of the gene, coding regions of the gene, non-coding regions of the gene, or any combination thereof.

In one embodiment, disclosed herein is a method for inducing an immune response against a tumor or a cancer in a human subject, the method comprising the step of administering to said subject a recombinant Listeria strain comprising a recombinant nucleic acid, said nucleic acid comprising a first open reading frame encoding a recombinant polypeptide comprising an N-terminal fragment of an LLO protein fused to a heterologous antigen or fragment thereof, is, wherein said recombinant nucleic acid further comprises a second open reading frame encoding a mutant PrfA protein, thereby inducing an immune response against a tumor or a cancer In one embodiment, the present invention provides a method of treating a cancer in a human subject, comprising the step of administering to the subject the recombinant Listeria strain disclosed herein. In another embodiment, the present invention provides a method of protecting a human subject against a cervical cancer, comprising the step of administering to the subject the recombinant Listeria strain disclosed herein. In another embodiment, the recombinant Listeria strain expresses the recombinant polypeptide. In another embodiment, the recombinant Listeria strain comprises a plasmid that encodes the recombinant polypeptide. In another embodiment, the method further comprises the step of boosting the human subject with a recombinant Listeria strain of the present invention. In another embodiment, the method further comprises the step of boosting the human subject with an immunogenic composition comprising a heterologous antigen or fragment thereof disclosed herein. In another embodiment, the method further comprises the step of boosting the human subject with an immunogenic composition that directs a cell of the subject to express the heterologous antigen. In another embodiment, the cell is a tumor cell. In another embodiment, the method further comprises the step of boosting the human subject with the vaccine of the present invention.

In one embodiment, the fragment thereof in the context of LLO proteins and ActA proteins disclosed herein refer to a peptide or polypeptide comprising an amino acid sequence of at least 5 contiguous amino acid residues of the LLO or ActA proteins. In another embodiment, the term refers to a peptide or polypeptide comprising an amino acid sequence of at least of at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, at least 250 contiguous amino acid residues of the amino acid sequence, at least 300 contiguous amino acid residues, at least 350 contiguous amino acid residues of, at least 400 contiguous amino acid residues, or at least 450 contiguous amino acid residues of an LLO or ActA protein or polypeptide.

In another embodiment, the fragment is a functional fragment that works as intended by the present invention (e.g. to elicit an immune response against a disease-associated antigen when in the form of an N-terminal LLO/heterologous antigen fusion protein or N-terminal ActA/heterologous antigen fusion protein). In another embodiment, the fragment is functional in a non-fused form. In another embodiment, the fragment is an immunogenic fragment.

The present invention, in certain embodiments, provides codon optimization of a nucleic acid heterologous to Listeria, or of a nucleic acid endogenous to Listeria. The optimal codons utilized by L. monocytogenes for each amino acid are shown US Patent Publication 2007/0207170, which is hereby incorporated by reference herein. A nucleic acid is codon-optimized if at least one codon in the nucleic acid is replaced with a codon that is more frequently used by L. monocytogenes for that amino acid than the codon in the original sequence.

As disclosed herein, recombinant Listeria strains expressing LLO-antigen fusions induce anti-tumor immunity (Example 1), elicit antigen-specific T cell proliferation (Example 2), generate antigen-specific, and tumor-infiltrating T cells (Example 3).

In another embodiment, the present invention provides a method of treating a cervical cancer in a human subject, comprising the step of administering to the subject a recombinant Listeria strain, the recombinant Listeria strain comprising a recombinant polypeptide comprising an N-terminal fragment of an LLO protein and an HPV E7 antigen, whereby the recombinant Listeria strain induces an immune response against the E7 antigen, thereby treating a cervical cancer in a human subject. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of protecting a human subject against an HPV-related cancer. In another embodiment, the present invention provides a method of protecting a human subject against a cervical cancer, comprising the step of administering to the subject a recombinant Listeria strain, the recombinant Listeria strain comprising a recombinant polypeptide comprising an N-terminal fragment of an LLO protein and at least one HPV antigen, whereby the recombinant Listeria strain induces an immune response against the HPV antigen, thereby protecting a human subject against a cervical cancer. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of inducing an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising an N-terminal fragment of an LLO protein and at least one HPV antigen, thereby inducing an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of treating an HPV-related cancer. In another embodiment, the present invention provides a method of treating a cervical cancer in a human subject, comprising the step of administering to the subject a recombinant Listeria strain, the recombinant Listeria strain comprising a recombinant polypeptide comprising an N-terminal fragment of an LLO protein and at least one HPV antigen, whereby the recombinant Listeria strain induces an immune response against the heterologous antigen, thereby treating a cervical cancer in a human subject. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of protecting a human subject against an HPV-related cancer. In another embodiment, the present invention provides a method of protecting a human subject against a cervical cancer, comprising the step of administering to the subject a recombinant Listeria strain, the recombinant Listeria strain comprising a recombinant polypeptide comprising an N-terminal fragment of an ActA protein and at least one HPV antigen, whereby the recombinant Listeria strain induces an immune response against the heterologous antigen, thereby protecting a human subject against a cervical cancer. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of inducing an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising an N-terminal fragment of an ActA protein and at least one heterologous antigen, thereby inducing an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide. In one embodiment, the present invention provides a method of treating an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising an N-terminal fragment of an ActA protein and at least one heterologous antigen, thereby treating an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of protecting a human subject against an HPV-related cancer. In another embodiment, the present invention provides a method of protecting a human subject against a cervical cancer, comprising the step of administering to the subject a recombinant Listeria strain, the recombinant Listeria strain comprising a recombinant polypeptide comprising a PEST sequence and at least one HPV antigen, whereby the recombinant Listeria strain induces an immune response against the heterologous antigen, thereby protecting a human subject against a cervical cancer. In another embodiment, the recombinant Listeria strain expresses the recombinant polypeptide. In another embodiment, the recombinant Listeria strain comprises a plasmid that encodes the recombinant polypeptide.

In one embodiment, the present invention provides a method of inducing an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising a PEST sequence and at least one heterologous antigen, thereby inducing an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide. In one embodiment, the present invention provides a method of treating an immune response against an HPV-related cancer. In another embodiment, the present invention provides a method for inducing an immune response against a cervical cancer in a human subject, comprising the step of administering to the subject a recombinant Listeria strain, the recombinant Listeria strain comprising a recombinant polypeptide comprising a PEST sequence and at least one heterologous antigen, thereby treating an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide.

The N-terminal ActA protein fragment and at least one heterologous antigen are, in another embodiment, fused directly to one another. In another embodiment, the genes encoding the N-terminal ActA protein fragment and at least one heterologous antigen are fused directly to one another. In another embodiment, the N-terminal ActA protein fragment and at least one heterologous antigen are attached via a linker peptide. In another embodiment, the N-terminal ActA protein fragment and at least one heterologous antigen are attached via a heterologous peptide. In another embodiment, the N-terminal ActA protein fragment is N-terminal to at least heterologous antigen. In another embodiment, the N-terminal ActA protein fragment is N-terminal to all of the heterologous antigens. In another embodiment, the N-terminal ActA protein fragment is the N-terminal-most portion of the fusion protein.

The PEST sequence and at least one heterologous antigen are, in another embodiment, fused directly to one another. In another embodiment, the genes encoding the PEST sequence and at least one heterologous antigen are fused directly to one another. In another embodiment, the PEST sequence and at least one heterologous antigen are attached via a linker peptide. In another embodiment, the PEST sequence and at least one heterologous antigen are attached via a heterologous peptide. In another embodiment, the PEST sequence is N-terminal to at least heterologous antigen. In another embodiment, the PEST sequence is N-terminal to all of the heterologous antigens. In another embodiment, the PEST sequence is the N-terminal-most portion of the fusion protein.

In another embodiment, the present invention provides a method for vaccinating a human subject against an HPV, comprising the step of administering to the subject the recombinant Listeria strain disclosed herein, wherein the Listeria expresses an HPV E7 antigen and wherein the Listeria expresses a mutant PrfA protein. In another embodiment, the mutant prfA gene encodes a D133V mutation in PrfA protein. In another embodiment, the mutant prfA gene is in a plasmid in said recombinant Listeria. In another embodiment, the recombinant Listeria strain expresses the recombinant polypeptide. In another embodiment, the recombinant Listeria strain comprises a plasmid that encodes the recombinant polypeptide.

In one embodiment, at least one of two to four heterologous antigens or functional fragments thereof disclosed herein is expressed from an extrachromosomal plasmid in said Listeria and at least one of two to four heterologous antigens or functional fragments thereof disclosed herein is expressed from the genome of said Listeria.

In one embodiment, the term “operably linked” as used herein means that the transcriptional and translational regulatory nucleic acid, is positioned relative to any coding sequences in such a manner that transcription is initiated. Generally, this will mean that the promoter and transcriptional initiation or start sequences are positioned 5′ to the coding region. In another embodiment the term “operably linked” as used herein means that several open reading frames are fused in a way that forms a single continuous reading frame resulting in expression of a protein that incorporates sequences of the original proteins arranged in succession.

In one embodiment, “fused” refers to operable linkage by covalent bonding. In one embodiment, the term includes recombinant fusion (of nucleic acid sequences or open reading frames thereof). In another embodiment, the term includes chemical conjugation.

In one embodiment the tag sequence comprises the tag sequence comprises a C-terminal SIINFEKL and 6 His amino acids. In another embodiment, the tag sequence is an amino acid or nucleic acid sequence that allows for easy detection of the fusion polypeptide. In another embodiment, the tag sequence is an amino acid or nucleic acid sequence that is useful for confirmation of secretion of a fusion polypeptide disclosed herein. It will be appreciated by a skilled artisan that the sequences for the tags may be incorporated into the fusion peptide sequences on the plasmid or phage vector. These tags may be expressed and the antigenic epitopes presented allow a clinician to follow the immunogenicity of the secreted peptide by following immune responses to these “tag” sequence peptides. Such immune response can be monitored using a number of reagents including but not limited to, monoclonal antibodies and DNA or RNA probes specific for these tags.

In one embodiment, a recombinant polypeptide disclosed herein is expressed and secreted by a recombinant Listeria disclosed herein. In another embodiment, secretion of the antigen, or polypeptides (fusion or chimeric) disclosed herein is detected using a protein, molecule or antibody (or fragment thereof) that specifically binds to a polyhistidine (His) tag. In another embodiment, the fusion polypeptide disclosed herein is expressed and secreted by a recombinant Listeria disclosed herein. In another embodiment, secretion of the antigen, or recombinant polypeptide disclosed herein is detected using an antibody, protein or molecule that binds a SIINFEKL-S-6×HIS tag. In another embodiment, the recombinant polypeptide disclosed herein comprise any other tag know in the art, including, but not limited to chitin binding protein (CBP), maltose binding protein (MBP), and glutathione-S-transferase (GST), thioredoxin (TRX) and poly(NANP).

In one embodiment, the heterologous antigen is any tumor associated antigen known in the art and disclosed herein. In another embodiment, the heterologous antigen is an autoimmune antigen. In another embodiment, the heterologous antigen is an infectious disease antigen. In another embodiment, the heterologous antigen is an HPV-related antigen.

The HPV that is the target of methods of the present invention is, in another embodiment, an HPV 16. In another embodiment, the HPV is an HPV-18. In another embodiment, the HPV is selected from HPV-16 and HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type.

In another embodiment, the present invention provides a method of vaccinating a human subject against a heterologus antigen, the method comprising the step of administering intravenously to the human subject a recombinant Listeria strain comprising or expressing the heterologus antigen, wherein the first peptide is selected from (a) an N-terminal fragment of an LLO protein; (b) an ActA protein or N-terminal fragment thereof; and (c) a PEST amino acid sequence-containing peptide, thereby vaccinating a human subject against a heterologus antigen.

In another embodiment, the present invention provides a method of vaccinating a human subject against a heterologus antigen, the method comprising the step of administering intravenously to the human subject an immunogenic composition, comprising a fusion of a first peptide to the heterologus antigen, wherein the first peptide is selected from (a) an N-terminal fragment of an LLO protein; (b) an ActA protein or N-terminal fragment thereof; and (c) a PEST amino acid sequence-containing peptide, thereby vaccinating a human subject against a heterologus antigen.

In another embodiment, the present invention provides a method of vaccinating a human subject against a heterologus antigen, the method comprising the step of administering intravenously to the human subject a recombinant Listeria strain comprising a recombinant polypeptide, the recombinant polypeptide comprising a first peptide fused to the heterologus antigen, wherein the first peptide is selected from (a) an N-terminal fragment of an LLO protein; (b) an ActA protein or N-terminal fragment thereof; and (c) a PEST amino acid sequence-containing peptide, thereby vaccinating a human subject against a heterologus antigen.

In another embodiment, the present invention provides a method of inducing a CTL response in a human subject against a heterologus antigen, the method comprising the step of administering to the human subject a recombinant Listeria strain comprising or expressing the heterologus antigen, thereby inducing a CTL response in a human subject against a heterologus antigen. In another embodiment, the step of administering is intravenous administration.

As disclosed herein, recombinant Listeria strains expressing LLO-antigen fusions induce anti-tumor immunity (Example 1), elicit antigen-specific T cell proliferation (Example 2), generate antigen-specific, and tumor-infiltrating T cells (Example 3). Thus, vaccines of the present invention are efficacious at inducing immune responses against E7 and E6.

In another embodiment, the present invention provides a method for inducing a regression of a cancer in a subject, comprising the step of administering to the subject the recombinant Listeria strain disclosed herein

In another embodiment, the present invention provides a method for reducing an incidence of relapse of a cancer in a subject, comprising the step of administering to the subject the recombinant Listeria strain disclosed herein.

In another embodiment, the present invention provides a method for suppressing a formation of a tumor in a subject, comprising the step of administering to the subject the recombinant Listeria strain disclosed herein.

In another embodiment, the present invention provides a method for inducing a remission of a cancer in a subject, comprising the step of administering to the subject the recombinant Listeria strain disclosed herein.

In another embodiment, the present invention provides a method for impeding a growth of a tumor in a human subject, comprising the step of administering to the subject the recombinant Listeria strain disclosed herein.

In another embodiment, the present invention provides a method for reducing a size of a tumor in a subject, comprising the step of administering to the subject the recombinant Listeria strain disclosed herein.

In one embodiment, the disease is an infectious disease, an autoimmune disease, a respiratory disease, a pre-cancerous condition or a cancer.

It will be well appreciated by the skilled artisan that the term “pre-cancerous condition” may encompass dysplasias, preneoplastic nodules; macroregenerative nodules (MRN); low-grade dysplastic nodules (LG-DN); high-grade dysplastic nodules (HG-DN); biliary epithelial dysplasia; foci of altered hepatocytes (FAH); nodules of altered hepatocytes (NAH); chromosomal imbalances; aberrant activation of telomerase; re-expression of the catalytic subunit of telomerase; expression of endothelial cell markers such as CD31, CD34, and BNH9 (see, e.g., Terracciano and Tomillo (2003) Pathologica 95:71-82; Su and Bannasch (2003) Toxicol. Pathol. 31:126-133; Rocken and Carl-McGrath (2001) Dig. Dis. 19:269-278; Kotoula, et al. (2002) Liver 22:57-69; Frachon, et al. (2001) J. Hepatol. 34:850-857; Shimonishi, et al. (2000) J. Hepatobiliary Pancreat. Surg. 7:542-550; Nakanuma, et al. (2003) J. Hepatobiliary Pancreat. Surg. 10:265-281). Methods for diagnosing cancer and dysplasia are disclosed (see, e.g., Riegler (1996) Semin. Gastrointest. Dis. 7:74-87; Benvegnu, et al. (1992) Liver 12:80-83; Giannini, et al. (1987) Hepatogastroenterol. 34:95-97; Anthony (1976) Cancer Res. 36:2579-2583).

In one embodiment, an infectious disease is one caused by, but not limited to, any one of the following pathogens: BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (HIN1) Pandemic, Measles and Rubella, Mumps, Meningococcus A+C, Oral Polio Vaccines, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, California encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Trichomonas vaginalis, or any other infectious disease known in the art that is not listed herein.

In another embodiment, the infectious disease is a livestock infectious disease. In another embodiment, livestock diseases can be transmitted to man and are called “zoonotic diseases.” In another embodiment, these diseases include, but are not limited to, Foot and mouth disease, West Nile Virus, rabies, canine parvovirus, feline leukemia virus, equine influenza virus, infectious bovine rhinotracheitis (IBR), pseudorabies, classical swine fever (CSF), IBR, caused by bovine herpesvirus type 1 (BHV-1) infection of cattle, and pseudorabies (Aujeszky's disease) in pigs, toxoplasmosis, anthrax, vesicular stomatitis virus, rhodococcus equi, Tularemia, Plague (Yersinia pestis), trichomonas.

In another embodiment, the disease disclosed herein is a respiratory or inflammatory disease. In another embodiment, the respiratory or inflammatory disease is chronic obstructive pulmonary disease (COPD). In another embodiment, the disease is asthma.

In one embodiment, live attenuated Listeria strains are capable of alleviating asthma symptoms without co-administration of other therapeutic agents, such as anti-inflammatory agents or bronchodilators. In another embodiment, the methods disclosed herein further comprise the step of co-administering to a subject the live attenuated Listeria strain and one or more therapeutic agents. In another embodiment, the therapeutic agent is an anti-asthmatic agent. In another embodiment, the agent is an anti-inflammatory agent, a non-steroidal anti-inflammatory agent, an antibiotic, an antichlolinerginc agent, a bronchodilator, a corticosteroid, a short-acting beta-agonist, a long-acting beta-agonist, combination inhalers, an antihistamine, or combinations thereof.

In one embodiment, a disease disclosed herein is a cancer or a tumor. In one embodiment, the tumor is cancerous. In another embodiment, the cancer is breast cancer. In another embodiment, the cancer is a cervical cancer. In another embodiment, the cancer is a Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, it is a glioblastoma multiforme. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is oropharyngeal cancer. In another embodiment, the cancer is lung cancer. In another embodiment, the cancer is anal cancer. In another embodiment, the cancer is lung cancer. In another embodiment, the cancer is vaginal cancer. In another embodiment, the cancer is colorectal cancer. In another embodiment, the cancer is esophageal cancer. The cervical tumor targeted by methods of the present invention is, in another embodiment, a squamous cell carcinoma. In another embodiment, the cervical tumor is an adenocarcinoma. In another embodiment, the cervical tumor is an adenosquamous carcinoma. In another embodiment, the cervical tumor is a small cell carcinoma. In another embodiment, the cervical tumor is any other type of cervical tumor known in the art.

In another embodiment, the compositions disclosed herein are useful for inducing an immune response against, preventing or treating a anal intracelluar neoplasia in a subject. In another embodiment, the compositions disclosed herein are useful for inducing an immune response against, preventing or treating a vaginal intracelluar neoplasia in a subject.

The cervical tumor targeted by methods of the present invention is, in another embodiment, a squamous cell carcinoma. In another embodiment, the cervical tumor is an adenocarcinoma. In another embodiment, the cervical tumor is an adenosquamous carcinoma. In another embodiment, the cervical tumor is a small cell carcinoma. In another embodiment, the cervical tumor is any other type of cervical tumor known in the art.

In one embodiment, an antigen may be foreign, that is, heterologous to the host and is referred to as a “heretologous antigen” herein. In another embodiment, the antigen is a self-antigen, which is an antigen that is present in the host but the host does not elicit an immune response against it because of immunologic tolerance. It will be appreciated by a skilled artisan that a heterologous antigen as well as a self-antigen may encompass a tumor antigen, a tumor-associated antigen or an angiogenic antigen. In addition, a heterologous antigen may encompass an infectious disease antigen.

In one embodiment, the antigen disclosed herein is a heterologous tumor antigen, which is also referred to herein as “tumor antigen” “antigenic polypeptide,” or “foreign antigen.” In another embodiment, the antigen disclosed herein is a self-antigen.

It will be appreciated by a skilled artisan that the term “heterologous” encompasses a nucleic acid, amino acid, peptide, polypeptide, or protein derived from a different species than the reference species. Thus, for example, a Listeria strain expressing a heterologous polypeptide, in one embodiment, would express a polypeptide that is not native or endogenous to the Listeria strain, or in another embodiment, a polypeptide that is not normally expressed by the Listeria strain, or in another embodiment, a polypeptide from a source other than the Listeria strain. In another embodiment, heterologous may be used to describe something derived from a different organism within the same species. In another embodiment, the heterologous antigen is expressed by a recombinant strain of Listeria, and is processed and presented to cytotoxic T-cells upon infection of mammalian cells by the recombinant strain. In another embodiment, the heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal. The term heterologous antigen may be referred to herein as “antigenic polypeptide”, “heterologous protein”, “heterologous protein antigen”, “protein antigen”, “antigen”, and the like.

In one embodiment, the antigen is Human Papilloma Virus-E7 (HPV-E7) antigen, which in one embodiment, is from HPV16 (in one embodiment, GenBank Accession No. AAD33253) and in another embodiment, from HPV18 (in one embodiment, GenBank Accession No. P06788). In another embodiment, the antigenic polypeptide is HPV-E6, which in one embodiment, is from HPV16 (in one embodiment, GenBank Accession No. AAD33252, AAM51854, AAM51853, or AAB67615) and in another embodiment, from HPV18 (in one embodiment, GenBank Accession No. P06463). In another embodiment, the antigenic polypeptide is a Her/2-neu antigen. In another embodiment, the antigenic polypeptide is Prostate Specific Antigen (PSA) (in one embodiment, GenBank Accession No. CAD30844, CAD54617, AAA58802, or NP-001639). In another embodiment, the antigenic polypeptide is Stratum Corneum Chymotryptic Enzyme (SCCE) antigen (in one embodiment, GenBank Accession No. AAK69652, AAK69624, AAG33360, AAF01139, or AAC37551). In another embodiment, the antigenic polypeptide is Wilms tumor antigen 1, which in another embodiment is WT-1 Telomerase (GenBank Accession. No. P49952, P22561, NP-659032, CAC39220.2, or EAW68222.1). In another embodiment, the antigenic polypeptide is hTERT or Telomerase (GenBank Accession. No. NM003219 (variant 1), NM198255 (variant 2), NM 198253 (variant 3), or NM 198254 (variant 4). In another embodiment, the antigenic polypeptide is Proteinase 3 (in one embodiment, GenBank Accession No. M29142, M75154, M96839, X55668, NM 00277, M96628 or X56606). In another embodiment, the antigenic polypeptide is Tyrosinase Related Protein 2 (TRP2) (in one embodiment, GenBank Accession No. NP-001913, ABI73976, AAP33051, or Q95119). In another embodiment, the antigenic polypeptide is High Molecular Weight Melanoma Associated Antigen (HMW-MAA) (in one embodiment, GenBank Accession No. NP-001888, AAI28111, or AAQ62842). In another embodiment, the antigenic polypeptide is Testisin (in one embodiment, GenBank Accession No. AAF79020, AAF79019, AAG02255, AAK29360, AAD41588, or NP-659206). In another embodiment, the antigenic polypeptide is NY-ESO-1 antigen (in one embodiment, GenBank Accession No. CAA05908, P78358, AAB49693, or NP-640343). In another embodiment, the antigenic polypeptide is PSCA (in one embodiment, GenBank Accession No. AAH65183, NP-005663, NP-082492, 043653, or CAB97347). In another embodiment, the antigenic polypeptide is Interleukin (IL) 13 Receptor alpha (in one embodiment, GenBank Accession No. NP-000631, NP-001551, NP-032382, NP-598751, NP-001003075, or NP_999506). In another embodiment, the antigenic polypeptide is Carbonic anhydrase IX (CAIX) (in one embodiment, GenBank Accession No. CAI13455, CAI10985, EAW58359, NP_001207, NP647466, or NP-001101426). In another embodiment, the antigenic polypeptide is carcinoembryonic antigen (CEA) (in one embodiment, GenBank Accession No. AAA66186, CAA79884, CAA66955, AAA51966, AAD15250, or AAA51970.). In another embodiment, the antigenic polypeptide is MAGE-A (in one embodiment, GenBank Accession No. NP_786885, NP_786884, NP-005352, NP-004979, NP-005358, or NP-005353). In another embodiment, the antigenic polypeptide is survivin (in one embodiment, GenBank Accession No. AAC51660, AAY15202, ABF60110, NP_001003019, or NP_001082350). In another embodiment, the antigenic polypeptide is GP100 (in one embodiment, GenBank Accession No. AAC60634, YP-655861, or AAB31176). In another embodiment, the antigenic polypeptide is any other antigenic polypeptide known in the art. In another embodiment, the antigenic peptide of the compositions and methods of the present invention comprise an immunogenic portion of the antigenic polypeptide.

In another embodiment, the antigen is HPV-E6. In another embodiment, the antigen is telomerase (TERT). In another embodiment, the antigen is LMP-1. In another embodiment, the antigen is p53. In another embodiment, the antigen is mesothelin. In another embodiment, the antigen is EGFRVIII. In another embodiment, the antigen is carboxic anhydrase IX (CAIX). In another embodiment, the antigen is PSMA. In another embodiment, the antigen is HMW-MAA. In another embodiment, the antigen is HIV-1 Gag. In another embodiment, the antigen is Tyrosinase related protein 2. In another embodiment, the antigen is selected from HPV-E7, HPV-E6, Her-2, HIV-1 Gag, LMP-1, p53, PSMA, carcinoembryonic antigen (CEA), LMP-1, kallikrein-related peptidase 3 (KLK3), KLK9, Muc, Tyrosinase related protein 2, Mucl, FAP, IL-13R alpha 2, PSA (prostate-specific antigen), gp-100, heat-shock protein 70 (HSP-70), beta-HCG, EGFR-III, Granulocyte colony-stimulating factor (G-CSF), Angiogenin, Angiopoietin-1, Del-1, Fibroblast growth factors: acidic (aFGF) or basic (bFGF), Follistatin, Granulocyte colony-stimulating factor (G-CSF), Hepatocyte growth factor (HGF)/scatter factor (SF), Interleukin-8 (IL-8), Leptin, Midkine, Placental growth factor, Platelet-derived endothelial cell growth factor (PD-ECGF), Platelet-derived growth factor-BB (PDGF-BB), Pleiotrophin (PTN), Progranulin, Proliferin, Transforming growth factor-alpha (TGF-alpha), Transforming growth factor-beta (TGF-beta), Tumor necrosis factor-alpha (TNF-alpha), Vascular endothelial growth factor (VEGF)/vascular permeability factor (VPF), VEGFR, VEGFR2 (KDR/FLK-1) or a fragment thereof, FLK-1 or an epitope thereof, FLK-E1, FLK-E2, FLK-I1, endoglin or a fragment thereof, Neuropilin 1 (NRP-1), Angiopoietin 1 (Angl), Tie2, Platelet-derived growth factor (PDGF), Platelet-derived growth factor receptor (PDGFR), Transforming growth factor-beta (TGF-β), endoglin, TGF-β receptors, monocyte chemotactic protein-1 (MCP-1), VE-cadherin, CD31, ephrin, ICAM-1, V-CAM-1, VAP-1, E-selectin, plasminogen activators, plasminogen activator inhibitor-1, Nitric oxide synthase (NOS), COX-2, AC133, or Id1/Id3, Angiopoietin 3, Angiopoietin 4, Angiopoietin 6, CD105, EDG, HHT1, ORW, ORW1 or a TGFbeta co-receptor, or a combination thereof. In another embodiment, the antigen is a chimeric Her2/neu antigen as disclosed in US Patent Application Publication No. 2011/0142791, which is incorporated by reference herein in its entirety. The use of fragments of antigens disclosed herein is also encompassed by the present invention.

In another embodiment, the heterologous tumor antigen disclosed herein is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 mclanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, a MART1 antigen associated with melanoma, or the PSA antigen associated with prostate cancer. In another embodiment, the antigen for the compositions and methods disclosed herein are melanoma-associated antigens, which in one embodiment are TRP-2, MAGE-1, MAGE-3, gp-100, tyrosinase, HSP-70, beta-HCG, or a combination thereof. It is to be understood that a skilled artisan would be able to use any heterologous antigen not mentioned herein but known in the art for use in the methods and compositions disclosed herein. It is also to be understood that the present invention provides, but is not limited by, an attenuated Listeria comprising a nucleic acid that encodes at least one of the antigens disclosed herein. The present invention encompasses nucleic acids encoding mutants, muteins, splice variants, fragments, truncated variants, soluble variants, extracellular domains, intracellular domains, mature sequences, and the like, of the disclosed antigens. Disclosed are nucleic acids encoding epitopes, oligo- and polypeptides of these antigens. Also disclosed are codon optimized embodiments, that is, optimized for expression in Listeria. The cited references, GenBank Acc. Nos., and the nucleic acids, peptides, and polypeptides disclosed herein, are all incorporated herein by reference in their entirety. In another embodiment, the selected nucleic acid sequence can encode a full length or a truncated gene, a fusion or tagged gene, and can be a cDNA, a genomic DNA, or a DNA fragment, preferably, a cDNA. It can be mutated or otherwise modified as desired. These modifications include codon optimizations to optimize codon usage in the selected host cell or bacteria, i.e. Listeria. The selected sequence can also encode a secreted, cytoplasmic, nuclear, membrane bound or cell surface polypeptide.

In one embodiment, vascular endothelial growth factor (VEGF) is an important signaling protein involved in both vasculogenesis (the formation of the embryonic circulatory system) and angiogenesis (the growth of blood vessels from pre-existing vasculature). In one embodiment, VEGF activity is restricted mainly to cells of the vascular endothelium, although it does have effects on a limited number of other cell types (e.g. stimulation monocyte/macrophage migration). In vitro, VEGF has been shown to stimulate endothelial cell mitogenesis and cell migration. VEGF also enhances microvascular permeability and is sometimes referred to as vascular permeability factor.

In one embodiment, all of the members of the VEGF family stimulate cellular responses by binding to tyrosine kinase receptors (the VEGFRs) on the cell surface, causing them to dimerize and become activated through transphosphorylation. The VEGF receptors have an extracellular portion consisting of 7 immunoglobulin-like domains, a single transmembrane spanning region and an intracellular portion containing a split tyrosine-kinase domain.

In one embodiment, VEGF-A is a VEGFR-2 (KDR/Flk-1) ligand as well as a VEGFR-1 (Flt-1) ligand. In one embodiment, VEGFR-mediates almost all of the known cellular responses to VEGF. The function of VEGFR-1 is less well defined, although it is thought to modulate VEGFR-2 signaling, in one embodiment, via sequestration of VEGF from VEGFR-2 binding, which in one embodiment, is particularly important during vasculogenesis in the embryo. In one embodiment, VEGF-C and VEGF-D are ligands of the VEGFR-3 receptor, which in one embodiment, mediates lymphangiogenesis.

In one embodiment, the compositions of the present invention comprise a VEGF receptor or a fragment thereof, which in one embodiment, is a VEGFR-2 and, in another embodiment, a VEGFR-1, and, in another embodiment, VEGFR-3.

In one embodiment, vascular Endothelial Growth Factor Receptor 2 (VEGFR2) is highly expressed on activated endothelial cells (ECs) and participates in the formation of new blood vessels. In one embodiment, VEGFR2 binds all 5 isoforms of VEGF. In one embodiment, signaling of VEGF through VEGFR2 on ECs induces proliferation, migration, and eventual differentiation. In one embodiment, the mouse homologue of VEGFR2 is the fetal liver kinase gene-1 (Flk-1), which is a strong therapeutic target, and has important roles in tumor growth, invasion, and metastasis. In one embodiment, VEGFR2 is also referred to as kinase insert domain receptor (a type III receptor tyrosine kinase) (KDR), cluster of differentiation 309 (CD309), FLK1, Ly73, Krd-1, VEGFR, VEGFR-2, or 6130401C07.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise, Mucl, or pSA.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough3 yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis.

In another embodiment, an HPV E6 antigen is utilized instead of or in addition to an E7 antigen in a method of the present invention for treating, protecting against, or inducing an immune response against a cervical cancer.

In another embodiment, an ActA protein fragment is utilized instead of or in addition to an LLO fragment in a method of the present invention for treating, protecting against, or inducing an immune response against a cervical cancer.

In another embodiment, a PEST amino acid sequence-containing protein fragment is utilized instead of or in addition to an LLO fragment in a method of the present invention for treating, protecting against, or inducing an immune response against a cervical cancer.

In another embodiment, the present invention provides an immunogenic composition comprising a recombinant Listeria of the present invention. In another embodiment, the immunogenic composition of methods and compositions of the present invention comprises a recombinant vaccine vector of the present invention. In another embodiment, the immunogenic composition comprises a plasmid of the present invention. In another embodiment, the immunogenic composition comprises an adjuvant. In one embodiment, a vector of the present invention may be administered as part of a vaccine composition.

In another embodiment, a vaccine of the present invention is delivered with an adjuvant. In one embodiment, the adjuvant favors a predominantly Th1-mediated immune response. In another embodiment, the adjuvant favors a Th1-type immune response. In another embodiment, the adjuvant favors a Th1-mediated immune response. In another embodiment, the adjuvant favors a cell-mediated immune response over an antibody-mediated response. In another embodiment, the adjuvant is any other type of adjuvant known in the art. In another embodiment, the immunogenic composition induces the formation of a T cell immune response against the target protein.

In another embodiment, the present invention provides a method for inducing an anti-E7 cytotoxic T cell (CTL) response in a human subject, comprising the step of administering to the subject a recombinant Listeria strain, the recombinant Listeria strain comprising a recombinant polypeptide comprising an N-terminal fragment of an LLO protein and an HPV E7 antigen, thereby inducing an anti-E7 CTL response in a human subject. In another embodiment, the recombinant Listeria strain comprises a plasmid that encodes the recombinant polypeptide. In another embodiment, the method further comprises the step of boosting the subject with a recombinant Listeria strain of the present invention. In another embodiment, the method further comprises the step of boosting the subject with an immunogenic composition comprising an E7 antigen. In another embodiment, the method further comprises the step of boosting the subject with an immunogenic composition that directs a cell of the subject to express an E7 antigen. In another embodiment, the CTL response is capable of therapeutic efficacy against an HPV-mediated disease, disorder, or symptom. In another embodiment, the CTL response is capable of prophylactic efficacy against an HPV-mediated disease, disorder, or symptom.

In another embodiment, the present invention provides a method of treating or ameliorating an HPV-mediated disease, disorder, or symptom in a subject, comprising the step of administering to the subject a recombinant Listeria strain, the recombinant Listeria strain comprising a recombinant polypeptide comprising an N-terminal fragment of an LLO protein and an HPV E7 antigen, whereby the recombinant Listeria strain induces an immune response against the E7 antigen, thereby treating or ameliorating an HPV-mediated disease, disorder, or symptom in a subject. In another embodiment, the subject is a human subject. In another embodiment, the subject is a non-human mammal. In another embodiment, the subject is any other type of subject known in the art.

The HPV causing the disease, disorder, or symptom is, in another embodiment, an HPV 16. In another embodiment, the HPV is an HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type.

In another embodiment, the HPV-mediated disease, disorder, or symptom is genital warts. In another embodiment, the HPV-mediated disease, disorder, or symptom is non-genital warts. In another embodiment, the HPV-mediated disease, disorder, or symptom is a respiratory papilloma. In another embodiment, the HPV-mediated disease, disorder, or symptom is any other HPV-mediated disease, disorder, or symptom known in the art.

In another embodiment, an HPV E6 antigen is utilized instead of or in addition to an E7 antigen in a method of the present invention for treating or ameliorating an HPV-mediated disease, disorder, or symptom.

In another embodiment, an ActA protein fragment is utilized instead of or in addition to an LLO fragment in a method of the present invention for treating or ameliorating an HPV-mediated disease, disorder, or symptom.

In another embodiment, a PEST amino acid sequence-containing protein fragment is utilized instead of or in addition to an LLO fragment in a method of the present invention for treating or ameliorating an HPV-mediated disease, disorder, or symptom.

In another embodiment, an HPV E6 antigen is utilized instead of or in addition to an E7 antigen in a method of the present invention for treating or ameliorating an HPV-mediated disease, disorder, or symptom.

The antigen of methods and compositions of the present invention is, in another embodiment, an HPV E7 protein. In another embodiment, the antigen is an HPV E6 protein. In another embodiment, the antigen is any other HPV protein known in the art.

“E7 antigen” refers, in another embodiment, to an E7 protein. In another embodiment, the term refers to an E7 fragment. In another embodiment, the term refers to an E7 peptide. In another embodiment, the term refers to any other type of E7 antigen known in the art.

The E7 protein of methods and compositions of the present invention is, in another embodiment, an HPV 16 E7 protein. In another embodiment, the E7 protein is an HPV-18 E7 protein. In another embodiment, the E7 protein is an HPV-31 E7 protein. In another embodiment, the E7 protein is an HPV-35 E7 protein. In another embodiment, the E7 protein is an HPV-39 E7 protein. In another embodiment, the E7 protein is an HPV-45 E7 protein. In another embodiment, the E7 protein is an HPV-51 E7 protein. In another embodiment, the E7 protein is an HPV-52 E7 protein. In another embodiment, the E7 protein is an HPV-58 E7 protein. In another embodiment, the E7 protein is an E7 protein of a high-risk HPV type. In another embodiment, the E7 protein is an E7 protein of a mucosal HPV type.

“E6 antigen” refers, in another embodiment, to an E6 protein. In another embodiment, the term refers to an E6 fragment. In another embodiment, the term refers to an E6 peptide. In another embodiment, the term refers to any other type of E6 antigen known in the art.

The E6 protein of methods and compositions of the present invention is, in another embodiment, an HPV 16 E6 protein. In another embodiment, the E6 protein is an HPV-18 E6 protein. In another embodiment, the E6 protein is an HPV-31 E6 protein. In another embodiment, the E6 protein is an HPV-35 E6 protein. In another embodiment, the E6 protein is an HPV-39 E6 protein. In another embodiment, the E6 protein is an HPV-45 E6 protein. In another embodiment, the E6 protein is an HPV-51 E6 protein. In another embodiment, the E6 protein is an HPV-52 E6 protein. In another embodiment, the E6 protein is an HPV-58 E6 protein. In another embodiment, the E6 protein is an E6 protein of a high-risk HPV type. In another embodiment, the E6 protein is an E6 protein of a mucosal HPV type.

The immune response induced by methods and compositions of the present invention is, in another embodiment, a T cell response. In another embodiment, the immune response comprises a T cell response. In another embodiment, the response is a CD8+ T cell response. In another embodiment, the response comprises a CD8+ T cell response.

In one embodiment, compositions of the present invention induce a strong innate stimulation of interferon-gamma, which in one embodiment, has anti-angiogenic properties. In one embodiment, a Listeria of the present invention induces a strong innate stimulation of interferon-gamma, which in one embodiment, has anti-angiogenic properties (Dominiecki et al., Cancer Immunol Immunother. 2005 May; 54(5):477-88. Epub 2004 Oct. 6, incorporated herein by reference in its entirety; Beatty and Paterson, J Immunol. 2001 Feb. 15; 166(4):2276-82, incorporated herein by reference in its entirety). In another embodiment, methods of the present invention increase a level of interferon-gamma producing cells. In one embodiment, anti-angiogenic properties of Listeria are mediated by CD4⁺ T cells (Beatty and Paterson, 2001). In another embodiment, anti-angiogenic properties of Listeria are mediated by CD8+ T cells. In another embodiment, IFN-gamma secretion as a result of Listeria vaccination is mediated by NK cells, NKT cells, Th1 CD4⁺ T cells, TC1 CD8⁺ T cells, or a combination thereof.

In another embodiment, compositions of the present invention induce production of one or more anti-angiogenic proteins or factors. In one embodiment, the anti-angiogenic protein is IFN-gamma. In another embodiment, the anti-angiogenic protein is pigment epithelium-derived factor (PEDF); angiostatin; endostatin; fms-like tyrosine kinase (sFlt)-1; or soluble endoglin (sEng). In one embodiment, a Listeria of the present invention is involved in the release of anti-angiogenic factors, and, therefore, in one embodiment, has a therapeutic role in addition to its role as a vector for introducing an antigen to a subject. Each Listeria strain and type thereof represents a separate embodiment of the present invention.

In another embodiment the immune response induced by methods and compositions of the present invention is suppression of programmed cell death receptor-1 ligand 1 (PD-L1) expression in the target tumor cells. In another embodiment, the immune response comprises increased level of programmed cell death receptor-1 (PD-1) expressing immune cells within tumor. In another embodiment, the immune response comprises increase in ratio of the level of PD-1 expression to PD-L1 expression. In another embodiment, the immune response comprises inhibition of tumor PD-L1-mediated immunosuppression.

In another embodiment, the administration of compositions of the present invention induces robust systemic antigen-specific immunity. In another embodiment, the administration of compositions of the present invention induces epitope spreading. In another embodiment, the administration of compositions of the present invention induces broad-based response to self-derived tumor antigens. In another embodiment the immune response induced by methods and compositions of the present invention comprises improvement of the overall balance of suppressor and effector immune cells in the tumor microenvironment (TME). In another embodiment the immune response induced by methods and compositions of the present invention comprises improvement in the systemic balance of suppressor and effector immunocytes.

In one embodiment, compositions and methods of use thereof as disclosed herein generate effector T cells that are able to infiltrate the tumor, destroy tumor cells and eradicate the disease. In another embodiment, methods of use of this invention increase umore infilatration by T effector cells. In another embodiment, T effector cells comprise CD8+ T cells. In another embodiment, T effector cells comprise CD4+ T cells.

In one embodiment, tumor infiltrating lymphocytes (TILs) are associated with better prognosis in several tumors, such as colon, ovarian and melanoma. In colon cancer, tumors without signs of micrometastasis have an increased infiltration of immune cells and a Th1 expression profile, which correlate with an improved survival of patients. Moreover, the infiltration of the tumor by T cells has been associated with success of immunotherapeutic approaches in both pre-clinical and human trials. In one embodiment, the infiltration of lymphocytes into the tumor site is dependent on the up-regulation of adhesion molecules in the endothelial cells of the tumor vasculature, generally by proinflammatory cytokines, such as IFN-γ, TNF-α and IL-1. Several adhesion molecules have been implicated in the process of lymphocyte infiltration into tumors, including intercellular adhesion molecule 1 (ICAM-1), vascular endothelial cell adhesion molecule 1 (V-CAM-1), vascular adhesion protein 1 (VAP-1) and E-selectin. However, these cell-adhesion molecules are commonly down-regulated in the tumor vasculature. Thus, in one embodiment, cancer vaccines as disclosed herein increase TILs, up-regulate adhesion molecules (in one embodiment, ICAM-1, V-CAM-1, VAP-1, E-selectin, or a combination thereof), up-regulate pro-inflammatory cytokines (in one embodiment, IFN-γ, TNF-α, IL-1, or a combination thereof), or a combination thereof.

The N-terminal LLO protein fragment of methods and compositions of the present invention comprises, in another embodiment, SEQ ID No: 2. In another embodiment, the fragment comprises an LLO signal peptide. In another embodiment, the fragment comprises SEQ ID No: 2. In another embodiment, the fragment consists approximately of SEQ ID No: 2. In another embodiment, the fragment consists essentially of SEQ ID No: 2. In another embodiment, the fragment corresponds to SEQ ID No: 2. In another embodiment, the fragment is homologous to SEQ ID No: 2. In another embodiment, the fragment is homologous to a fragment of SEQ ID No: 2. The ALLO used in some of the Examples was 416 AA long (exclusive of the signal sequence), as 88 residues from the amino terminus which is inclusive of the activation domain containing cysteine 484 were truncated. It will be clear to those skilled in the art that any ALLO without the activation domain, and in particular without cysteine 484, are suitable for methods and compositions of the present invention. In another embodiment, fusion of an E7 or E6 antigen to any ALLO, including the PEST amino acid AA sequence, SEQ ID NO: 1, enhances cell mediated and anti-tumor immunity of the antigen.

The LLO protein utilized to construct vaccines of the present invention has, in another embodiment, the sequence:

MKKIMLVFITLILVSLPIAQQTEAKDASAFNKENSISSMAPPASPPASPKTPIEKKHA DEIDKYIQGLDYNKNNVLVYHGDAVTNVPPRKGYKDGNEYIVVEKKKKSINQNNAD IQVVNAISSLTYPGALVKANSELVENQPDVLPVKRDSLTLSIDLPGMTNQDNKIVVKN ATKSNVNNAVNTLVERWNEKYAQAYPNVSAKIDYDDEMAYSESQLIAKFGTAFKAV NNSLNVNFGAISEGKMQEEVISFKQIYYNVNVNEPTRPSRFFGKAVTKEQLQALGVN AENPPAYISSVAYGRQVYLKLSTNSHSTKVKAAFDAAVSGKSVSGDVELTNIIKNSSF KAVIYGGSAKDEVQIIDGNLGDLRDILKKGATFNRETPGVPIAYTTNFLKDNELAVIK NNSEYIETTSKAYTDGKINIDHSGGYVAQFNISWDEVNYDPEGNEIVQHKNWSENNK SKLAHFTSSIYLPGNARNINVYAKECTGLAWEWWRTVIDDRNLPLVKNRNISIWGTT LYPKYSNKVDNPIE (GenBank Accession No. P13128; SEQ ID NO: 3; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine of the present invention.

In another embodiment, the N-terminal fragment of an LLO protein utilized in compositions and methods of the present invention has the sequence:

(SEQ ID NO: 2)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYD.

In another embodiment, the LLO fragment corresponds to about AA 20-442 of an LLO protein utilized herein.

In another embodiment, the LLO fragment has the sequence:

(SEQ ID NO: 4)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTD.

In another embodiment, “truncated LLO” or “ALLO” refers to a fragment of LLO that comprises the PEST amino acid domain. In another embodiment, the terms refer to an LLO fragment that comprises a PEST sequence.

In another embodiment, the terms refer to an LLO fragment that does not contain the activation domain at the amino terminus and does not include cysteine 484. In another embodiment, the terms refer to an LLO fragment that is not hemolytic. In another embodiment, the LLO fragment is rendered non-hemolytic by deletion or mutation of the activation domain. In another embodiment, the LLO fragment is rendered non-hemolytic by deletion or mutation of cysteine 484. In another embodiment, the LLO fragment is rendered non-hemolytic by deletion or mutation at another location.

In another embodiment, the LLO fragment consists of about the first 441 AA of the LLO protein. In another embodiment, the LLO fragment consists of about the first 420 AA of LLO. In another embodiment, the LLO fragment is a non-hemolytic form of the LLO protein.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly.

In another embodiment, the LLO fragment is any other LLO fragment known in the art.

The recombinant Listeria strain of methods and compositions of the present invention is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

The present invention provides a number of Listerial species and strains for making or engineering an attenuated Listeria of the present invention. In one embodiment, the Listeria strain is L. monocytogenes 10403S wild type (see Bishop and Hinrichs (1987) J. Immunol. 139: 2005-2009; Lauer, et al. (2002) J. Bact. 184: 4177-4186.) In another embodiment, the Listeria strain is L. monocytogenes DP-L4056 (phage cured) (see Lauer, et al. (2002) J. Bact. 184: 4177-4186). In another embodiment, the Listeria strain is L. monocytogenes DP-L4027, which is phage cured and deleted in the hly gene (see Lauer, et al. (2002) J. Bact. 184: 4177-4186; Jones and Portnoy (1994) Infect. Immunity 65: 5608-5613.). In another embodiment, the Listeria strain is L. monocytogenes DP-L4029, which is phage cured, deleted in ActA (see Lauer, et al. (2002) J. Bact. 184: 4177-4186; Skoble, et al. (2000) J. Cell Biol. 150: 527-538). In another embodiment, the Listeria strain is L. monocytogenes DP-L4042 (delta PEST) (see Brockstedt, et al. (2004) Proc. Natl. Acad. Sci. USA 101: 13832-13837; supporting information). In another embodiment, the Listeria strain is L. monocytogenes DP-L4097 (LLO-S44A) (see Brockstedt, et al. (2004) Proc. Natl. Acad. Sci. USA 101: 13832-13837; supporting information). In another embodiment, the Listeria strain is L. monocytogenes DP-L4364 (delta lplA; lipoate protein ligase) (see Brockstedt, et al. (2004) Proc. Natl. Acad. Sci. USA 101: 13832-13837; supporting information). In another embodiment, the Listeria strain is L. monocytogenes DP-L4405 (delta inlA) (see Brockstedt, et al. (2004) Proc. Natl. Acad. Sci. USA 101: 13832-13837; supporting information). In another embodiment, the Listeria strain is L. monocytogenes DP-L4406 (delta inlB) (see Brockstedt, et al. (2004) Proc. Natl. Acad. Sci. USA 101: 13832-13837; supporting information). In another embodiment, the Listeria strain is L. monocytogenes CS-L0001 (delta ActA-delta inlB) (see Brockstedt, et al. (2004) Proc. Natl. Acad. Sci. USA 101: 13832-13837; supporting information). In another embodiment, the Listeria strain is L. monocytogenes CS-L0002 (delta ActA-delta lplA) (see Brockstedt, et al. (2004) Proc. Natl. Acad. Sci. USA 101: 13832-13837; supporting information). In another embodiment, the Listeria strain is L. monocytogenes CS-L0003 (L461T-delta lplA) (see Brockstedt, et al. (2004) Proc. Natl. Acad. Sci. USA 101: 13832-13837; supporting information). In another embodiment, the Listeria strain is L. monocytogenes DP-L4038 (delta ActA-LLO L461T) (see Brockstedt, et al. (2004) Proc. Natl. Acad. Sci. USA 101: 13832-13837; supporting information). In another embodiment, the Listeria strain is L. monocytogenes DP-L4384 (S44A-LLO L461T) (see Brockstedt, et al. (2004) Proc. Natl. Acad. Sci. USA 101: 13832-13837; supporting information). In another embodiment, the Listeria strain is L. monocytogenes. Mutation in lipoate protein (see O'Riordan, et al. (2003) Science 302: 462-464). In another embodiment, the Listeria strain is L. monocytogenes DP-L4017 (10403S hly (L461T), having a point mutation in hemolysin gene (see U.S. Provisional Pat. Appl. Ser. No. 60/490,089 filed Jul. 24, 2003). In another embodiment, the Listeria strain is L. monocytogenes EGD (see GenBank Acc. No. AL591824). In another embodiment, the Listeria strain is L. monocytogenes EGD-e (see GenBank Acc. No. NC_003210. ATCC Acc. No. BAA-679). In another embodiment, the Listeria strain is L. monocytogenes DP-L4029 deleted in uvrAB (see U.S. Provisional Pat. Appl. Ser. No. 60/541,515 filed Feb. 2, 2004; U.S. Provisional Pat. Appl. Ser. No. 60/490,080 filed Jul. 24, 2003). In another embodiment, the Listeria strain is L. monocytogenes ActA-/inlB—double mutant (see ATCC Acc. No. PTA-5562). In another embodiment, the Listeria strain is L. monocytogenes lplA mutant or hly mutant (see U.S. Pat. Applic. No. 20040013690 of Portnoy, et. al). In another embodiment, the Listeria strain is L. monocytogenes DAIJDAT double mutant. (see U.S. Pat. Applic. No. 20050048081 of Frankel and Portnoy. The present invention encompasses reagents and methods that comprise the above Listerial strains, as well as these strains that are modified, e.g., by a plasmid and/or by genomic integration, to contain a nucleic acid encoding one of, or any combination of, the following genes: hly (LLO; listeriolysin); iap (p60); inlA; inlB; inlC; dal (alanine racemase); dat (D-amino acid aminotransferase); plcA; plcB; actA; or any nucleic acid that mediates growth, spread, breakdown of a single walled vesicle, breakdown of a double walled vesicle, binding to a host cell, uptake by a host cell. The present invention is not to be limited by the particular strains disclosed above.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein (e.g. in Example 12). In another embodiment, the passaging is performed by any other method known in the art.

In another embodiment, the recombinant Listeria strain utilized in methods of the present invention has been stored in a frozen cell bank. In another embodiment, the recombinant Listeria strain has been stored in a lyophilized cell bank.

In another embodiment, the cell bank of methods and compositions of the present invention is a master cell bank. In another embodiment, the cell bank is a working cell bank. In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States.

In another embodiment, a recombinant Listeria strain utilized in methods of the present invention is from a batch of vaccine doses.

In another embodiment, a recombinant Listeria strain utilized in methods of the present invention is from a frozen or lyophilized stock produced by methods disclosed in U.S. Pat. No. 8,114,414, which is incorporated by reference herein.

In another embodiment, a peptide of the present invention is a fusion peptide. In another embodiment, “fusion peptide” refers to a peptide or polypeptide comprising 2 or more proteins linked together by peptide bonds or other chemical bonds. In another embodiment, the proteins are linked together directly by a peptide or other chemical bond. In another embodiment, the proteins are linked together with 1 or more AA (e.g. a “spacer”) between the 2 or more proteins.

In another embodiment, a vaccine of the present invention further comprises an adjuvant. The adjuvant utilized in methods and compositions of the present invention is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art.

In another embodiment, a nucleotide of the present invention is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide of the present invention is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter/regulatory sequence, which is either known or unknown, and which is capable of driving expression of the desired protein operably linked thereto.

An N-terminal fragment of an ActA protein utilized in methods and compositions of the present invention has, in another embodiment, the sequence set forth in SEQ ID NO: 5:

MRAMMVVFITANCITINPDIIFAATDSEDSSLNTDEWEEEKTEEQPSEVNTGPRYE TAREVSSRDIKELEKSNKVRNTNKADLIAMLKEKAEKGPNINNNNSEQTENAAINEEA SGADRPAIQVERRHPGLPSDSAAEIKKRRKAIASSDSELESLTYPDKPTKVNKKKVAK ESVADASESDLDSSMQSADESSPQPLKANQQPFFPKVFKKIKDAGKWVRDKIDENPE VKKAIVDKSAGLIDQLLTKKKSEEVNASDFPPPPTDEELRLALPETPMLLGFNAPATSE PSSFEFPPPPTDEELRLALPETPMLLGFNAPATSEPSSFEFPPPPTEDELEIIRETASSLDSS FTRGDLASLRNAINRHSQNFSDFPPIPTEEELNGRGGRP. In another embodiment, the ActA fragment comprises the sequence set forth in SEQ ID NO: 5. In another embodiment, the ActA fragment is any other ActA fragment known in the art.

In another embodiment, the recombinant nucleotide encoding a fragment of an ActA protein comprises the sequence set forth in SEQ ID NO: 6:

Atgcgtgcgatgatggtggttttcattactgccaattgcattacgattaaccccgacataatatttgcagcgacagatagcgaagatt ctagtctaaacacagatgaatgggaagaagaaaaaacagaagagcaaccaagcgaggtaaatacgggaccaagatacgaaactgca cgtgaagtaagttcacgtgatattaaagaactagaaaaatcgaataaagtgagaaatacgaacaaagcagacctaatagcaatgttgaa agaaaaagcagaaaaaggtccaaatatcaataataacaacagtgaacaaactgagaatgcggctataaatgaagaggcttcaggagc cgaccgaccagctatacaagtggagcgtcgtcatccaggattgccatcggatagcgcagcggaaattaaaaaaagaaggaaagccat agcatcatcggatagtgagcttgaaagccttacttatccggataaaccaacaaaagtaaataagaaaaaagtggcgaaagagtcagttg cggatgcttctgaaagtgacttagattctagcatgcagtcagcagatgagtcttcaccacaacctttaaaagcaaaccaacaaccatttttc cctaaagtatttaaaaaaataaaagatgcggggaaatgggtacgtgatgacgaaaatcctgaagtaaagaaagcgattgttgat aaaagtgcagggttaattgaccaattattaaccaaaaaagaaaagtgaagaggtaaatgcttcggacttcccgccaccacctacggatga agagttaagacttgctttgccagagacaccaatgcttcttggttttaatgctcctgctacatcagaaccgagctcattcgaatttccaccacc acctacggatgaagagttaagacttgctttgccagagacgccaatgcttcttggttttaatgctacatcgagagctcgttcg aatttccaccgcctccaacagaagatgaactagaaatcatccgggaaacagcatcctcgctagattctagttttacaagaggggatttag ctagtttgagaaatgctattaatcgccatagtcaaaatttctctgatttcccaccaatcccaacagaagaagagttgaacgggagaggcg gtagacca. In another embodiment, the recombinant nucleotide has the sequence set forth in SEQ ID NO: 6. In another embodiment, the recombinant nucleotide comprises any other sequence that encodes a fragment of an ActA protein.

In another embodiment of the methods and compositions of the present invention, a PEST amino acid AA sequence is fused to the E7 or E6 antigen. As disclosed herein, recombinant Listeria strains expressing PEST amino acid sequence-antigen fusions induce anti-tumor immunity (Example 3) and generate antigen-specific, tumor-infiltrating T cells (Example 4). Further, enhanced cell mediated immunity was demonstrated for fusion proteins comprising an antigen and LLO containing the PEST amino acid AA sequence

(SEQ ID NO: 1)
KENSISSMAPPASPPASPKTPIEKKHADEIDK.

Thus, fusion of an antigen to other LM PEST amino acid sequences and PEST amino acid sequences derived from other prokaryotic organisms will also enhance immunogenicity of the antigen. The PEST amino acid AA sequence has, in another embodiment, a sequence selected from SEQ ID NO: 7-12. In another embodiment, the PEST amino acid sequence is a PEST amino acid sequence from the LM ActA protein. In another embodiment, the PEST amino acid sequence is KTEEQPSEVNTGPR (SEQ ID NO: 7), KASVTDTSEGDLDSSMQSADESTPQPLK (SEQ ID NO: 8), KNEEVNASDFPPPPTDEELR (SEQ ID NO: 9), or RGGIPTSEEFSSLNSGDFTDDENSETTEEEIDR (SEQ ID NO: 10). In another embodiment, the PEST amino acid sequence is from Streptolysin O protein of Streptococcus sp. In another embodiment, the PEST amino acid sequence is from Streptococcus pyogenes Streptolysin O, e.g. KQNTASTETTTTNEQPK (SEQ ID NO: 11) at AA 35-51. In another embodiment, the PEST amino acid sequence is from Streptococcus equisimilis Streptolysin O, e.g. KQNTANTETTTTNEQPK (SEQ ID NO:12) at AA 38-54. In another embodiment, the PEST amino acid sequence is another PEST amino acid AA sequence derived from a prokaryotic organism. In another embodiment, the PEST amino acid sequence is any other PEST amino acid sequence known in the art.

PEST amino acid sequences of other prokaryotic organism can be identified in accordance with methods such as described by, for example Rechsteiner and Rogers (1996, Trends Biochem. Sci. 21:267-271) for LM. Alternatively, PEST amino acid AA sequences from other prokaryotic organisms can also be identified based by this method. Other prokaryotic organisms wherein PEST amino acid AA sequences would be expected to include, but are not limited to, other Listeria species. In another embodiment, the PEST amino acid sequence is embedded within the antigenic protein. Thus, in another embodiment, “fusion” refers to an antigenic protein comprising both the antigen and the PEST amino acid amino acid sequence either linked at one end of the antigen or embedded within the antigen.

In another embodiment, the PEST amino acid sequence is identified using any other method or algorithm known in the art, e.g the CaSPredictor (Garay-Malpartida H M, Occhiucci J M, Alves J, Belizario J E. Bioinformatics. 2005 June; 21 Suppl 1:i169-76). In another embodiment, the following method is used:

A PEST index is calculated for each 30-35 AA stretch by assigning a value of 1 to the amino acids Ser, Thr, Pro, Glu, Asp, Asn, or Gln. The coefficient value (CV) for each of the PEST residue is 1 and for each of the other AA (non-PEST) is 0.

In another embodiment, the LLO protein, ActA protein, or fragment thereof of the present invention need not be that which is set forth exactly in the sequences set forth herein, but rather other alterations, modifications, or changes can be made that retain the functional characteristics of an LLO or ActA protein fused to an antigen as set forth elsewhere herein. In another embodiment, the present invention utilizes an analog of an LLO protein, ActA protein, or fragment thereof. Analogs differ, in another embodiment, from naturally occurring proteins or peptides by conservative AA sequence differences or by modifications which do not affect sequence, or by both.

In another embodiment, either a whole E7 protein or a fragment thereof is fused to a LLO protein, ActA protein, or PEST amino acid sequence-containing peptide to generate a recombinant peptide of methods of the present invention. The E7 protein that is utilized (either whole or as the source of the fragments) has, in another embodiment, the sequence MHGDTPTLHEYMLDLQPETTDLYCYEQLNDSSEEEDEIDGPAGQAEPDRAHYNIVTF CCKCDSTLRLCVQSTHVDIRTLEDLLMGTLGIVCPICSQKP (SEQ ID No: 13). In another embodiment, the E7 protein is a homologue of SEQ ID No: 13. In another embodiment, the E7 protein is a variant of SEQ ID No: 13. In another embodiment, the E7 protein is an isomer of SEQ ID No: 13. In another embodiment, the E7 protein is a fragment of SEQ ID No: 13. In another embodiment, the E7 protein is a fragment of a homologue of SEQ ID No: 13. In another embodiment, the E7 protein is a fragment of a variant of SEQ ID No: 13. In another embodiment, the E7 protein is a fragment of an isomer of SEQ ID No: 13.

In another embodiment, the sequence of the E7 protein is: MHGPKATLQDIVLHLEPQNEIPVDLLCHEQLSDSEEENDEIDGVNHQHLPARRAEPQR HTMLCMCCKCEARIELVVESSADDLRAFQQLFLNTLSFVCPWCASQQ (SEQ ID No: 14). In another embodiment, the E7 protein is a homologue of SEQ ID No: 14. In another embodiment, the E7 protein is a variant of SEQ ID No: 14. In another embodiment, the E7 protein is an isomer of SEQ ID No: 14. In another embodiment, the E7 protein is a fragment of SEQ ID No: 14. In another embodiment, the E7 protein is a fragment of a homologue of SEQ ID No: 14. In another embodiment, the E7 protein is a fragment of a variant of SEQ ID No: 14. In another embodiment, the E7 protein is a fragment of an isomer of SEQ ID No: 14.

In another embodiment, the E7 protein has a sequence set forth in one of the following GenBank entries: M24215, NC_004500, V01116, X62843, or M14119. In another embodiment, the E7 protein is a homologue of a sequence from one of the above GenBank entries. In another embodiment, the E7 protein is a variant of a sequence from one of the above GenBank entries. In another embodiment, the E7 protein is an isomer of a sequence from one of the above GenBank entries. In another embodiment, the E7 protein is a fragment of a sequence from one of the above GenBank entries. In another embodiment, the E7 protein is a fragment of a homologue of a sequence from one of the above GenBank entries. In another embodiment, the E7 protein is a fragment of a variant of a sequence from one of the above GenBank entries. In another embodiment, the E7 protein is a fragment of an isomer of a sequence from one of the above GenBank entries.

In another embodiment, either a whole E6 protein or a fragment thereof is fused to a LLO protein, ActA protein, or PEST amino acid sequence-containing peptide to generate a recombinant peptide of methods of the present invention. The E6 protein that is utilized (either whole or as the source of the fragments) has, in another embodiment, the sequence MHQKRTAMFQDPQERPRKLPQLCTELQTTIHDIILECVYCKQQLLRREVYDFAFRDLC IVYRDGNPYAVCDKCLKFYS KISEYRHYCYS LYGTTLEQQYNKPLCDLLIRCINCQKP LCPEEKQRHLDKKQRFHNIRGRWTGRCMSCCRSSRTRRETQL (SEQ ID No: 15). In another embodiment, the E6 protein is a homologue of SEQ ID No: 15. In another embodiment, the E6 protein is a variant of SEQ ID No: 15. In another embodiment, the E6 protein is an isomer of SEQ ID No: 15. In another embodiment, the E6 protein is a fragment of SEQ ID No: 15. In another embodiment, the E6 protein is a fragment of a homologue of SEQ ID No: 15. In another embodiment, the E6 protein is a fragment of a variant of SEQ ID No: 15. In another embodiment, the E6 protein is a fragment of an isomer of SEQ ID No: 15.

In another embodiment, the sequence of the E6 protein is: MARFEDPTRRPYKLPDLCTELNTSLQDIEITCVYCKTVLELTEVFEFAFKDLFVVYRDS IPHAACHKCIDFYSRIRELRHYSDSVYGDTLEKLTNTGLYNLLIRCLRCQKPLNPAEKL RHLNEKRRFHNIAGHYRGQCHSCCNRARQERLQRRRETQV (SEQ ID No: 16). In another embodiment, the E6 protein is a homologue of SEQ ID No: 16. In another embodiment, the E6 protein is a variant of SEQ ID No: 16. In another embodiment, the E6 protein is an isomer of SEQ ID No: 16. In another embodiment, the E6 protein is a fragment of SEQ ID No: 16. In another embodiment, the E6 protein is a fragment of a homologue of SEQ ID No: 16. In another embodiment, the E6 protein is a fragment of a variant of SEQ ID No: 16. In another embodiment, the E6 protein is a fragment of an isomer of SEQ ID No: 16.

In another embodiment, the E6 protein has a sequence set forth in one of the following GenBank entries: M24215, M14119, NC_004500, V01116, X62843, or M14119. In another embodiment, the E6 protein is a homologue of a sequence from one of the above GenBank entries. In another embodiment, the E6 protein is a variant of a sequence from one of the above GenBank entries. In another embodiment, the E6 protein is an isomer of a sequence from one of the above GenBank entries. In another embodiment, the E6 protein is a fragment of a sequence from one of the above GenBank entries. In another embodiment, the E6 protein is a fragment of a homologue of a sequence from one of the above GenBank entries. In another embodiment, the E6 protein is a fragment of a variant of a sequence from one of the above GenBank entries. In another embodiment, the E6 protein is a fragment of an isomer of a sequence from one of the above GenBank entries.

In another embodiment, “homology” refers to identity to an LLO sequence (e.g. to one of SEQ ID No: 2-4) of greater than 70%. In another embodiment, “homology” refers to identity to one of SEQ ID No: 2-4 of greater than 64%. In another embodiment, “homology” refers to identity to one of SEQ ID No: 2-4 of greater than 68%. In another embodiment, “homology” refers to identity to one of SEQ ID No: 2-4 of greater than 72%. In another embodiment, “homology” refers to identity to one of SEQ ID No: 2-4 of greater than 75%. In another embodiment, “homology” refers to identity to one of SEQ ID No: 2-4 of greater than 78%. In another embodiment, “homology” refers to identity to one of SEQ ID No: 2-4 of greater than 80%. In another embodiment, “homology” refers to identity to one of SEQ ID No: 2-4 of greater than 82%. In another embodiment, “homology” refers to identity to one of SEQ ID No: 2-4 of greater than 83%. In another embodiment, “homology” refers to identity to one of SEQ ID No: 2-4 of greater than 85%. In another embodiment, “homology” refers to identity to one of SEQ ID No: 2-4 of greater than 87%. In another embodiment, “homology” refers to identity to one of SEQ ID No: 2-4 of greater than 88%. In another embodiment, “homology” refers to identity to one of SEQ ID No: 2-4 of greater than 90%. In another embodiment, “homology” refers to identity to one of SEQ ID No: 2-4 of greater than 92%. In another embodiment, “homology” refers to identity to one of SEQ ID No: 2-4 of greater than 93%. In another embodiment, “homology” refers to identity to one of SEQ ID No: 2-4 of greater than 95%. In another embodiment, “homology” refers to identity to one of SEQ ID No: 2-4 of greater than 96%. In another embodiment, “homology” refers to identity to one of SEQ ID No: 2-4 of greater than 97%. In another embodiment, “homology” refers to identity to one of SEQ ID No: 2-4 of greater than 98%. In another embodiment, “homology” refers to identity to one of SEQ ID No: 2-4 of greater than 99%. In another embodiment, “homology” refers to identity to one of SEQ ID No: 2-4 of 100%.

In another embodiment, “homology” refers to identity to an E7 sequence (e.g. to one of SEQ ID No: 13-14) of greater than 70%. In another embodiment, “homology” refers to identity to one of SEQ ID No: 13-14 of greater than 62%. In another embodiment, “homology” refers to identity to one of SEQ ID No: 13-14 of greater than 64%. In another embodiment, “homology” refers to identity to one of SEQ ID No: 13-14 of greater than 68%. In another embodiment, “homology” refers to identity to one of SEQ ID No: 13-14 of greater than 72%. In another embodiment, “homology” refers to identity to one of SEQ ID No: 13-14 of greater than 75%. In another embodiment, “homology” refers to identity to one of SEQ ID No: 13-14 of greater than 78%. In another embodiment, “homology” refers to identity to one of SEQ ID No: 13-14 of greater than 80%. In another embodiment, “homology” refers to identity to one of SEQ ID No: 13-14 of greater than 82%. In another embodiment, “homology” refers to identity to one of SEQ ID No: 13-14 of greater than 83%. In another embodiment, “homology” refers to identity to one of SEQ ID No: 13-14 of greater than 85%. In another embodiment, “homology” refers to identity to one of SEQ ID No: 13-14 of greater than 87%. In another embodiment, “homology” refers to identity to one of SEQ ID No: 13-14 of greater than 88%. In another embodiment, “homology” refers to identity to one of SEQ ID No: 13-14 of greater than 90%. In another embodiment, “homology” refers to identity to one of SEQ ID No: 13-14 of greater than 92%. In another embodiment, “homology” refers to identity to one of SEQ ID No: 13-14 of greater than 93%. In another embodiment, “homology” refers to identity to one of SEQ ID No: 13-14 of greater than 95%. In another embodiment, “homology” refers to identity to one of SEQ ID No: 13-14 of greater than 96%. In another embodiment, “homology” refers to identity to one of SEQ ID No: 13-14 of greater than 97%. In another embodiment, “homology” refers to identity to one of SEQ ID No: 13-14 of greater than 98%. In another embodiment, “homology” refers to identity to one of SEQ ID No: 13-14 of greater than 99%. In another embodiment, “homology” refers to identity to one of SEQ ID No: 13-14 of 100%.

In another embodiment, “homology” refers to identity to an E6 sequence (e.g. to one of SEQ ID No: 15-16) of greater than 70%. In another embodiment, “homology” refers to identity to one of SEQ ID No: 15-16 of greater than 64%. In another embodiment, “homology” refers to identity to one of SEQ ID No: 15-16 of greater than 68%. In another embodiment, “homology” refers to identity to one of SEQ ID No: 15-16 of greater than 72%. In another embodiment, “homology” refers to identity to one of SEQ ID No: 15-16 of greater than 75%. In another embodiment, “homology” refers to identity to one of SEQ ID No: 15-16 of greater than 78%. In another embodiment, “homology” refers to identity to one of SEQ ID No: 15-16 of greater than 80%. In another embodiment, “homology” refers to identity to one of SEQ ID No: 15-16 of greater than 82%. In another embodiment, “homology” refers to identity to one of SEQ ID No: 15-16 of greater than 83%. In another embodiment, “homology” refers to identity to one of SEQ ID No: 15-16 of greater than 85%. In another embodiment, “homology” refers to identity to one of SEQ ID No: 15-16 of greater than 87%. In another embodiment, “homology” refers to identity to one of SEQ ID No: 15-16 of greater than 88%. In another embodiment, “homology” refers to identity to one of SEQ ID No: 15-16 of greater than 90%. In another embodiment, “homology” refers to identity to one of SEQ ID No: 15-16 of greater than 92%. In another embodiment, “homology” refers to identity to one of SEQ ID No: 15-16 of greater than 93%. In another embodiment, “homology” refers to identity to one of SEQ ID No: 15-16 of greater than 95%. In another embodiment, “homology” refers to identity to one of SEQ ID No: 15-16 of greater than 96%. In another embodiment, “homology” refers to identity to one of SEQ ID No: 15-16 of greater than 97%. In another embodiment, “homology” refers to identity to one of SEQ ID No: 15-16 of greater than 98%. In another embodiment, “homology” refers to identity to one of SEQ ID No: 15-16 of greater than 99%. In another embodiment, “homology” refers to identity to one of SEQ ID No: 15-16 of 100%.

In another embodiment, “homology” refers to identity to a PEST amino acid sequence (e.g. to one of SEQ ID No: 1, and 7-12) or to an ActA sequence (e.g. to one of SEQ ID No: 5-6) of greater than 70%. In another embodiment, “homology” refers to identity to one of SEQ ID No: 1, and 7-12 or SEQ ID No: 5-6 of greater than 60%. In another embodiment, “homology” refers to identity to one of SEQ ID No: 1, and 7-12 or SEQ ID No: 5-6 of greater than 64%. In another embodiment, “homology” refers to identity to one of SEQ ID No: 1, and 7-12 or SEQ ID No: 5-6 of greater than 68%. In another embodiment, “homology” refers to identity to one of SEQ ID No: 1, and 7-12 or SEQ ID No: 5-6 of greater than 72%. In another embodiment, “homology” refers to identity to one of SEQ ID No: 1, and 7-12 or SEQ ID No: 5-6 of greater than 75%. In another embodiment, “homology” refers to identity to one of SEQ ID No: 1, and 7-12 or SEQ ID No: 5-6 of greater than 78%. In another embodiment, “homology” refers to identity to one of SEQ ID No: 1, and 7-12 or SEQ ID No: 5-6 of greater than 80%. In another embodiment, “homology” refers to identity to one of SEQ ID No: 1, and 7-12 or SEQ ID No: 5-6 of greater than 82%. In another embodiment, “homology” refers to identity to one of SEQ ID No: 1, and 7-12 or SEQ ID No: 5-6 of greater than 83%. In another embodiment, “homology” refers to identity to one of SEQ ID No: 1, and 7-12 or SEQ ID No: 5-6 of greater than 85%. In another embodiment, “homology” refers to identity to one of SEQ ID No: 1, and 7-12 or SEQ ID No: 5-6 of greater than 87%. In another embodiment, “homology” refers to identity to one of SEQ ID No: 1, and 7-12 or SEQ ID No: 5-6 of greater than 88%. In another embodiment, “homology” refers to identity to one of SEQ ID No: 1, and 7-12 or SEQ ID No: 5-6 of greater than 90%. In another embodiment, “homology” refers to identity to one of SEQ ID No: 1, and 7-12 or SEQ ID No: 5-6 of greater than 92%. In another embodiment, “homology” refers to identity to one of SEQ ID No: 1, and 7-12 or SEQ ID No: 5-6 of greater than 93%. In another embodiment, “homology” refers to identity to one of SEQ ID No: 1, and 7-12 or SEQ ID No: 5-6 of greater than 95%. In another embodiment, “homology” refers to identity to one of SEQ ID No: 1, and 7-12 or SEQ ID No: 5-6 of greater than 96%. In another embodiment, “homology” refers to identity to one of SEQ ID No: 1, and 7-12 or SEQ ID No: 5-6 of greater than 97%. In another embodiment, “homology” refers to identity to one of SEQ ID No: 1, and 7-12 or SEQ ID No: 5-6 of greater than 98%. In another embodiment, “homology” refers to identity to one of SEQ ID No: 1, and 7-12 or SEQ ID No: 5-6 of greater than 99%. In another embodiment, “homology” refers to identity to one of SEQ ID No: 1, and 7-12 or SEQ ID No: 5-6 of 100%.

Protein and/or peptide homology for any AA sequence listed herein is determined, in one embodiment, by methods well described in the art, including immunoblot analysis, or via computer algorithm analysis of AA sequences, utilizing any of a number of software packages available, via established methods. Some of these packages include the FASTA, BLAST, MPsrch or Scanps packages, and employ, in other embodiments, the use of the Smith and Waterman algorithms, and/or global/local or BLOCKS alignments for analysis, for example.

In another embodiment, the LLO protein, ActA protein, or fragment thereof is attached to the antigen by chemical conjugation. In another embodiment, glutaraldehyde is used for the conjugation. In another embodiment, the conjugation is performed using any suitable method known in the art.

In another embodiment, fusion proteins of the present invention are prepared by any suitable method, including, for example, cloning and restriction of appropriate sequences or direct chemical synthesis by methods discussed below. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid.

In another embodiment, the LLO protein, ActA protein, or fragment thereof and the antigen, or fragment thereof are conjugated by a means known to those of skill in the art. In another embodiment, the antigen, or fragment thereof is conjugated, either directly or through a linker (spacer), to the ActA protein or LLO protein. In another embodiment, the chimeric molecule is recombinantly expressed as a single-chain fusion protein.

In another embodiment, a fusion peptide of the present invention is synthesized using standard chemical peptide synthesis techniques. In another embodiment, the chimeric molecule is synthesized as a single contiguous polypeptide. In another embodiment, the LLO protein, ActA protein, or fragment thereof; and the antigen, or fragment thereof are synthesized separately, then fused by condensation of the amino terminus of one molecule with the carboxyl terminus of the other molecule, thereby forming a peptide bond. In another embodiment, the ActA protein or LLO protein and antigen are each condensed with one end of a peptide spacer molecule, thereby forming a contiguous fusion protein.

In another embodiment, the peptides and proteins of the present invention are prepared by solid-phase peptide synthesis (SPPS) as described by Stewart et al. in Solid Phase Peptide Synthesis, 2nd Edition, 1984, Pierce Chemical Company, Rockford, Ill.; or as described by Bodanszky and Bodanszky (The Practice of Peptide Synthesis, 1984, Springer-Verlag, New York). In another embodiment, a suitably protected AA residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial AA, and couple thereto of the carboxyl end of the next AA in the sequence of the desired peptide. This AA is also suitably protected. The carboxyl of the incoming AA can be activated to react with the N-terminus of the support-bound AA by formation into a reactive group such as formation into a carbodiimide, a symmetric acid anhydride or an “active ester” group such as hydroxybenzotriazole or pentafluorophenly esters.

In another embodiment, the present invention provides a kit comprising vaccine of the present invention, an applicator, and instructional material that describes use of the methods of the invention. Although model kits are described below, the contents of other useful kits will be apparent to the skilled artisan in light of the present disclosure. Each of these kits represents a separate embodiment of the present invention.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, pets mice and humans. The subject may also include livestock. In one embodiment, the term “subject” does not exclude an individual that is healthy in all respects and does not have or show signs of disease or disorder.

CD137 (4-1BB)

CD137 (4-1BB) or TNF receptor superfamily member 9 (TNFRSF9) is a costimulatory receptor that belongs to the tumor necrosis factor (TNF) receptor family. CD137 regulates many immune cells, including CD4⁺ and CD8+ T cells, regulatory T cells (Treg), dendritic cells (DC), and natural (NK) cells. CD137 is expressed on NK. DC and T cells, and can potentiate antitumor responses by altering the cellular make-up of the TME (Makkoutk A., et al., “Rationale for anti-CD137 cancer immunotherapy”. European J Cancer, 2016, 54, 112-119).

CTLA-4 (CD152)

CTLA-4 or CD152 is a protein receptor that functions as an immune checkpoint and binds to B7-1 (CD80) and B7-2 (CD86) on antigen presenting cells (APCs).

Anti-CD137 (4-188) Agonist Antibody

Examples of mAbs that bind to human CD137, and useful in the treatment method, medicaments and uses of the present disclosure, are described in U.S. Pat. Nos. 9,382,238, 8,137,667, 7,659,384, 7,288,638, and 8,716,452. Specific anti-human CD137 mAbs useful in the treatment method, medicaments and uses of the present disclosure include: utomilumab, a fully humanized agonistic mAb and urelumab, a humanized agonistic mAb. Urelumab specifically binds to and activates CD137-expressing immune cells, stimulating an immune response, in particular a cytoxic T cell response, against tumor cells

Anti-CTLA-4 (CD152) Blocking Antibody

Examples of mAbs that bind to human CTLA-4, and useful in the treatment method, medicaments and uses of the present disclosure, are described in U.S. Pat. Nos. 7,919,079, 6,984,720, 7,605,238, 8,017,114, 7,034,121. Specific anti-human CTLA-4 mAbs useful in the treatment method, medicaments and uses of the present disclosure include: ipilimumab, a fully human IgG1 mAb and tremelimumab, fully human IgG2 mAb.

Anti-CTLA-4 mAbs block the binding of the APC ligands B7-1 and B7-2 to CTLA-4 resulting in inhibition of B7-CTLA-4 mediated downregulation of T-cell activation.

Antibodies

It will be appreciated by a skilled artisan an antibody that “specifically binds to” a specified target protein may encompass an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies or binding fragments thereof, useful in the present disclosure will bind to the target protein with an affinity that is at least two fold greater, preferably at least ten times greater, more preferably at least 20-times greater, and most preferably at least 100-times greater than the affinity with non-target proteins. In one embodiment, an antibody is said to bind specifically to a polypeptide comprising a given amino acid sequence, e.g. the amino acid sequence of a mature human CTLA-4, or human CD137, if it binds to polypeptides comprising that sequence but does not bind to proteins lacking that sequence.

In one embodiment, compositions disclosed herein comprise an antibody or a functional fragment thereof, which specifically binds CTLA-4 or a portion thereof. In another embodiment, compositions disclosed herein comprise an antibody or functional fragment thereof, which specifically binds CD137 or a portion thereof. In another embodiment, a composition may comprise an antibody that specifically bind CTLA-4 or a portion thereof, and an antibody that specifically binds CD137. In another embodiment, a composition of this invention comprises an Lm strain and an antibody or a functional fragment thereof that specifically binds CTLA-4. In another embodiment, a composition of this invention comprises an Lm strain and an antibody or a functional fragment thereof that specifically binds CD137. In another embodiment, a composition of this invention comprises an Lm strain and an antibody that specifically binds CTLA-4 or a portion thereof, and an antibody that specifically binds CD137 or a portion thereof. In another embodiment, a composition of this invention comprises an antibody or a functional fragment thereof that specifically binds CTLA-4, wherein the composition does not include a Listeria strain disclosed herein. In another embodiment, a composition of this invention comprises an antibody or a functional fragment thereof that specifically binds CD137, wherein the composition does not include a Listeria strain disclosed herein. In another embodiment, a composition of this invention comprises an antibody or a functional fragment thereof that specifically binds CTLA-4, and an antibody that specifically binds CTLA-4, wherein the composition does not include a Listeria strain disclosed herein. Different antibodies present in the same or different compositions need not have the same form, for example one antibody may be a monoclonal antibody and another may be a FAb fragment.

A skilled artisan will understand that the term “synthetic antibody” may encompass an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In one embodiment, an antibody or functional fragment thereof comprises an antigen binding region. In one embodiment, an antigen binding regions is an antibody or an antigen-binding domain thereof. In one embodiment, the antigen-binding domain thereof is a Fab or a scFv.

It will be appreciated by a skilled artisan that the term “binds” or “specifically binds,” with respect to an antibody, encompasses an antibody or functional fragment thereof, which recognizes a specific antigen, but does not substantially recognize or bind other molecules in a sample. For example, an antibody that specifically binds to an antigen from one species may also bind to that antigen from one or more species, but, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than a specific amino acid sequence.

The term “antibody functional fragment” refers to a portion of an intact antibody that is capable of specifically binding to an antigen to cause the biological effect intended by the present invention. Examples of antibody fragments include, but are not limited to, Fab, Fab′, F(ab′)2, and Fv fragments, linear antibodies, scFv antibodies, and multispecific antibodies formed from antibody fragments.

As used herein, the term “antibody” includes intact immunoglobulin molecules comprising 4 polypeptide chains, two heavy (H) chains and two light (L) chains inter-connected by disulfide bonds. Each heavy chain is comprised of a heavy chain variable region (abbreviated herein as VH) and a heavy chain constant region. The heavy chain constant region contains three domains, CH1, CH2 and CH3. Each light chain is comprised of a light chain variable region (abbreviated herein as VL) and a light chain constant region. The light chains of antibodies from any vertebrate species can be assigned to one of two clearly distinct types, called kappa (κ) and lambda (.), based on the amino acid sequences of their constant domains. The variable regions of kappa light chains are referred to herein as VK. The expression of VL, as used herein, is intended to include both the variable regions from kappa-type light chains (VK) and from lambda-type light chains. The light chain constant region is comprised of one domain, CL. The VH and VL regions include regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1-CDR1-FR2-CDR2-FR3-CDR3-FR4. “CDRH1” refers to the first CDR region in an antibody heavy chain, “CDRH2” refers to the second CDR region in an antibody heavy chain, and “CDRH3” refers to the third CDR region in an antibody heavy chain. “CDRL1” refers to the first CDR region in an antibody light chain, “CDRL2” refers to the second CDR region in an antibody light chain, and “CDRL3” refers to the third CDR region in an antibody light chain.

Depending on the amino acid sequence of the constant domain of their heavy chains, antibodies can be assigned to different classes. There are 5 major classes of intact antibodies: IgA, IgD, IgE, IgG, and IgM. Several of these may be further divided into subclasses (isotypes), e.g., IgG1, IgG2, IgG3, IgG4, IgA, and IgA2. The heavy-chain constant domains that correspond to the different classes of antibodies are called alpha (α), delta (Δ), epsilon (ε), gamma (γ), and mu (μ), respectively. The subunit structures and three-dimensional configurations of different classes of immunoglobulins are well known. The present disclosure includes antibodies of any of the aforementioned classes or subclasses (isotypes).

It will be appreciated by a skilled artisan that the term “Variable regions” or “V region” may encompass the segment of IgG chains which is variable in sequence between different antibodies. It extends to Kabat residue 109 in the light chain and 113 in the heavy chain. A “variable region” of an antibody refers to the variable region of the antibody light chain or the variable region of the antibody heavy chain, either alone or in combination. Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

It will be appreciated by a skilled artisan that the term “hypervariable region” may encompass the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). As used herein, the term “framework” or “FR” residues refers to variable domain residues other than the hypervariable region residues defined herein as CDR residues.

It will be appreciated by a skilled artisan that the term “Framework region” or “FR” may encompass the immunoglobulin variable regions excluding the CDR regions.

The term “antibody” as used herein is also intended to encompass intact antibodies, functional fragments which bind antigen, and variants thereof which bind antigen, including antibody mimetics or comprising portions of antibodies that mimic the structure and/or function of an antibody or specified fragment or portion thereof; each containing at least one CDR. Antibodies of the disclosure include antibody fragments or variants having one, two, three, four, five, six or more CDR regions.

Antibody fragments which are embraced by the present disclosure include Fab (e.g., by papain digestion), Fab′, F(ab′)₂. facb (e.g., by plasmin digestion), pFc′ (e.g., by pepsin or plasmin digestion), Fd (e.g., by pepsin digestion, partial reduction and reaggregation), sVds, and Fv or scFv (e.g., by molecular biology techniques). Antibody fragments are also intended to include domain deleted antibodies, diabodies, triabodies, linear antibodies, single-chain antibody molecules (including camelized antibodies), and multispecific antibodies formed from antibody fragments.

The term “antibody,” as used herein, may also include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species (e.g., mouse or rat) or belonging to another antibody class or subclass, as well as fragments of such antibodies, so long as they exhibit the desired biological activity. Thus, the present disclosure includes, for example, chimeric antibodies comprising a chimeric heavy chain and/or a chimeric light chain. The chimeric heavy chain may comprise any of the heavy chain variable (VH) regions described herein or mutants or variants thereof fused to a heavy chain constant region of a non-human antibody. The chimeric light chain may comprise any of the light chain variable (VL) regions described herein or mutants or variants thereof fused to a light chain constant region of a non-human antibody.

Antibodies of the disclosure also include “humanized antibodies”, which are antibody molecules having one or more complementarity determining regions (CDRs) from a non-human species and framework regions from a human immunoglobulin molecule. Often, framework residues in the human framework regions will be substituted with the corresponding residue from the CDR donor antibody to alter, or improve, antigen binding. These framework substitutions are identified standard techniques such as by modeling of the interactions of the CDR and framework residues to identify framework residues important for antigen binding and sequence comparison to identify unusual framework residues at particular positions. Antibodies can be humanized using a variety of techniques including CDR-grafting, veneering or resurfacing, and chain shuffling.

It will be appreciated by a skilled artisan that the term “human antibody,” includes antibodies having variable and constant regions corresponding to human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs. The human antibody can have at least one position replaced with an amino acid residue, e.g., an activity enhancing amino acid residue which is not encoded by the human germline immunoglobulin sequence. However, the term “human antibody,” as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences.

The term “monoclonal antibody,” as used herein, refers to an antibody obtained from a population of substantially homogeneous antibodies, e.g., the individual antibodies comprising the population are substantially identical except for possible naturally occurring mutations or minor post-translational variations that may be present. Monoclonal antibodies are highly specific, being directed against a single antigenic site (also known as determinant or epitope). Furthermore, in contrast to conventional (polyclonal) antibody preparations which typically include different antibodies directed against different determinants, each monoclonal antibody is directed against a single determinant on the antigen. The modifier “monoclonal” indicates the character of the antibody as being obtained from a substantially homogeneous population of antibodies, and is not to be construed as requiring production of the antibody by any particular method.

Antibodies also include polypeptides with amino acid sequences substantially similar to the amino acid sequence of the variable or hypervariable regions of the heavy and/or light chain. Substantially the same amino acid sequence is defined herein as a sequence with at least 70%, 75%, 80%, 85%, 90%, 95%, or 99% identity to a compared amino acid sequence, as determined by the FASTA search method in accordance with Pearson and Lipman, Proc. Natl. Acad. Sci. USA 85:2444-2448 (1988).

Antibodies of the disclosure include those which are identical to those described herein except with one or more conservative amino acid substitutions. Conservative amino acid substitution is defined as a change in the amino acid composition by way of changing one or two amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter peptide, polypeptide or protein characteristics (e.g., charge, isoelectric point, affinity, avidity, conformation, solubility) or activity. Typical substitutions that may be performed for such conservative amino acid substitution may be among the groups of amino acids as follows: glycine (G), alanine (A), valine (V), leucine (L) and isoleucine (I); aspartic acid (D) and glutamic acid (E); alanine (A), serine (S) and threonine (T); histidine (H), lysine (K) and arginine (R); asparagine (N) and glutamine (Q); phenylalanine (F), tyrosine (Y) and tryptophan (W).

Conservative amino acid substitutions can be made in the CDR or framework regions, e.g., regions flanking the hypervariable regions primarily responsible for the selective and/or specific binding characteristics of the molecule, as well as other parts of the molecule, e.g., variable heavy chain cassette.

Antibodies of the present disclosure also include those having their affinity increased or altered by direct mutation, affinity maturation, phage display, or chain shuffling. Affinity and specificity can be modified or improved by mutating CDR and/or framework residues and screening for antigen binding sites having the desired characteristics. One way is to randomize individual residues or combinations of residues so that in a population of, otherwise identical antigen binding sites, subsets of from two to twenty amino acids are found at particular positions. Alternatively, mutations can be induced over a range of residues by error using PCR methods. In another example, phage display vectors containing heavy and light chain variable region genes can be propagated in mutated strains of E. coli.

Antibodies prepared by chain shuffling, include those where the heavy or light chain are randomly paired with other heavy or light chains described herein. Thus, the antibodies of the disclosure include any combination of heavy and light chains (either full length or portions thereof).

The antibodies of the present disclosure are specific for antigens.

Specificity of the antibodies can be determined based on affinity and/or avidity. Affinity, represented by the equilibrium constant for the dissociation of an antigen with an antibody (K_{d}), measures the binding strength between an antigenic determinant and an antibody-binding site. Avidity is the measure of the strength of binding between an antibody with its antigen. Avidity is related to both the affinity between an epitope with its antigen binding site on the antibody, and the valence of the antibody, which refers to the number of antigen binding sites of a particular epitope. Antibodies typically bind with a dissociation constant (K_{d}) of about 10⁻⁵ to about 10⁻¹¹ liters/mol (e.g., K_{D}<100 nM). Any K_{d} less than about 10⁻⁴ liters/mol is generally considered to indicate nonspecific binding. The lesser the value of the K_{d}, the stronger the binding strength between an antigenic determinant and the antibody binding site.

The antibodies of the disclosure bind to its antigen with a K_{d} of preferably about 1×10⁻⁸ M⁻¹ or less, more preferably about 1×10⁻⁹ M⁻¹ or less, more preferably about 1×10⁻¹⁰ M⁻¹ or less, and most preferably about 1×10⁻¹¹ M⁻¹ or less.

Antibodies of the present disclosure can be monospecific, bispecific or multispecific. Monospecific antibodies bind to only one antigen. Bispecific antibodies (BsAbs) are antibodies that have two different antigen-binding specificities or sites. Multispecific antibodies have more than two different antigen-binding specificities or sites. Where an antibody has more than one specificity, the recognized epitopes can be associated with a single antigen or with more than one antigen.

In another aspect of the disclosure, the antibody is conjugated to another moiety, either directly or indirectly. The conjugation may be chemical or biosynthetic. Other moieties which can be conjugated to the antibodies include toxins, anti-tumor agents, detectable labels, target moieties and reporter moieties. Suitable toxins are described herein.

Antibodies which are conjugated to detectable labels can be used, for example, to diagnose a disease, to aid in prognosis and to locate tumor cells, in vivo or in vitro. The detectable label produces a measurable signal which is detectable by external means. Detectable labels include an enzyme, a chromophore, a radioisotope, or a substance that emits light by fluorescence, phosphorescence or chemiluminescence. Suitable enzymes include horseradish peroxidase, alkaline phosphatase, j3-galactosidase, and acetylcholinesterase. Chromophores include dyes which absorb light in the ultraviolet or visible region, and can be substrates or degradation products of enzyme catalyzed reactions. Suitable fluorescent materials include umbelliferone, fluorescein, fluorescein isothiocyanate, rhodamine, dichlorotriazinylamine fluorescein, dansyl chloride or phycoerythrin. Suitable chemiluminescence materials include luminol, luciferase, luciferin, and aequorin. Suitable radioactive materials include 1251, 1311, 35S, and 3H.

Target moieties are first members of binding pairs. Anti-tumor agents, for example, may be conjugated to second members of such pairs and are thereby directed to the site where the antigen-binding protein is bound. A common example of such a binding pair is avidin and biotin. Biotin may be conjugated to an antibody of the disclosure, thereby providing a target for an anti-tumor agent or other moiety which is conjugated to avidin or streptavidin. Alternatively, biotin or another such moiety is linked to an antigen-binding protein of the disclosure and used as a reporter, for example, a detectable label is conjugated to avidin or streptavidin.

In one embodiment, compositions disclosed herein comprise an antibody or a functional fragment thereof. In another embodiment, the compositions comprise at least one antibody or functional fragment thereof. In another embodiment, a composition may comprise 2 antibodies, 3 antibodies, 4 antibodies, or more than 4 antibodies. In another embodiment, a composition of this invention comprises an Lm strain and an antibody or a functional fragment thereof. In another embodiment, a composition disclosed herein comprises an Lm strain and at least one antibody or a functional fragment thereof. In another embodiment, a composition disclosed herein comprises an Lm strain and 2 antibodies, 3 antibodies, 4 antibodies, or more than 4 antibodies. In another embodiment, a composition disclosed herein comprises an antibody or a functional fragment thereof. Different antibodies present in the same or different compositions need not have the same form, for example one antibody may be a monoclonal antibody and another may be a FAb fragment.

The present disclosure also includes nucleic acid molecules that encode an antibody or portion thereof. Nucleic acids may encode an antibody heavy chain, comprising any one of the VH regions or a portion thereof, or any one of the VH CDRs, including any variants thereof, as disclosed herein. The disclosure also includes nucleic acid molecules that encode an antibody light chain comprising any one of the VL regions or a portion thereof or any one of the VL CDRs, including any variants thereof as disclosed herein. In certain embodiments, the nucleic acid encodes both a heavy and light chain, or portions thereof.

The disclosure also includes recombinant vectors comprising any of the nucleic acid molecules described herein. The vector may comprise a nucleic acid encoding only one antibody chain or a portion thereof (e.g., the heavy or light chain) or a nucleic acid encoding both antibody chains or portions thereof.

Exemplary vectors include plasmids, phagemids, cosmids, viruses and phage nucleic acids or other nucleic acid molecules that are capable of replication in a prokaryotic or eukaryotic host. The vectors typically contain a marker to provide a phenotypic trait for selection of transformed hosts such as conferring resistance to antibiotics such as ampicillin or neomycin

The vector may be an expression vector, wherein the nucleic acid encoding the antibody is operably linked to an expression control sequence. Typical expression vectors contain transcription and translation terminators, initiation sequences, and promoters useful for regulation of the expression of the nucleic acid molecules of the disclosure. The vectors may also contain genetic expression cassettes containing an independent terminator sequence, sequences permitting replication of the vector in both eukaryotes and prokaryotes, i.e., shuttle vectors and selection markers for both prokaryotic and eukaryotic systems. When the vector contains nucleic acids encoding both a heavy and light chain or portions thereof, the nucleic acid encoding the heavy chain may be under the same or a separate promoter. The separate promoters may be identical or may be different types of promoters.

Suitable promoters include constitutive promoters and inducible promoters. Representative expression control sequences/promoters include the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., PhoS, the promoters of the yeast alpha mating factors, promoters derived from the human cytomegalovirus, metallothionine promoter, murine mammary tumor virus promoter, Rous sarcoma virus promoter, polyhedrin promoter and promoters derived from polyoma, adenovirus, retrovirus, and simian virus, e.g., the early and late promoters of SV40.

The disclosure also includes non-human hosts such as cells or organisms containing a nucleic acid molecule or a vector of the disclosure. By “host” it is meant a non-human unicellular or multicellular organism or a “host cell”, which refers to a cell or population of cells into which a nucleic acid molecule or vector of the disclosure is introduced. “A population of host cells” refers to a group of cultured cells into which a nucleic acid molecule or vector of the present disclosure can be introduced and expressed. The host contain a nucleic acid or vector encoding only one chain or portion thereof (e.g., the heavy or light chain); or it may contain a nucleic acid or vector encoding both chains or portions thereof, either the same or separate nucleic acids and/or vectors.

A host of the present disclosure may be prokaryotic or eukaryotic. Suitable prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces. Suitable eukaryotic cells include yeast and other fungi, insect cells, plant cells, human cells, and animal cells, including mammalian cells, such as hybridoma lines, COS cells, NSO cells and CHO cells.

The disclosure also includes methods of producing an antibody of the present disclosure, which entails culturing a host cell expressing one or more nucleic acid sequences encoding an antibody of the present disclosure, and recovering the antibody from the culture medium. In certain embodiments, the antibody is purified by separating it from the culture medium. Antibodies comprising more than one chain can be produced by expressing each chain together in the same host; or as separate chains, which are assembled before or after recovery from the culture medium.

The disclosure also provides a pharmaceutical composition comprising the antibody, nucleic acid, vector, host cell, or chemotherapy agents of this disclosure and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

The pharmaceutical compositions of the disclosure may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In some embodiments, the compositions are in the form of injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject disclosure, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringeability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in US Appl. Publ. No. 2002/0102208 A1, which is incorporated herein by reference in its entirety. Such articles of manufacture will preferably have labels or package inserts indicating that the associated compositions are useful for treating a subject suffering from, or predisposed to autoimmune or neoplastic disorders.

Effective doses of the compositions of the present disclosure, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

The pharmaceutical compositions of the disclosure may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented.

Cancers/tumors which may be treated by the disclosure include any cancer or tumor. Examples of cancers/tumors which may be treated include, but are not limited to, breast cancer (including HER2+ and metastatic)

In a particular embodiment, cancers/tumors which may be treated by the disclosure is a triple negative breast cancer (TNBC).

Methods of treating cancer include, but are not limited to, e.g., inhibiting angiogenesis in the tumor, inhibiting tumor growth, inhibiting tumor migration, inhibiting proliferation or inhibiting invasion of tumor cells.

Cancers to be treated include primary tumors and secondary or metastatic tumors (including those metastasized from lung, breast, or prostate), as well as recurrent or refractory tumors. Recurrent tumors encompass tumors that appear to be inhibited by treatment with such agents, but recur up to five years, sometimes up to ten years or longer after treatment is discontinued. Refractory tumors are tumors that have failed to respond or are resistant to treatment with one or more conventional therapies for the particular tumor type. Refractory tumors include those that are hormone-refractory (e.g., androgen-independent prostate cancer; or hormone-refractory breast cancer, such as breast cancer that is refractory to tamoxifen); those that are refractory to treatment with one or more chemotherapeutic agents; those that are refractory to radiation; and those that are refractory to combinations of chemotherapy and radiation, chemotherapy and hormone therapy, or hormone therapy and radiation

Therapy may be “first-line”, i.e., as an initial treatment in patients who have had no prior anti-cancer treatments, either alone or in combination with other treatments; or “second-line”, as a treatment in patients who have had one prior anti-cancer treatment regimen, either alone or in combination with other treatments; or as “third-line”, “fourth-line”, etc. treatments, either alone or in combination with other treatments.

Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of tumor.

Cancers that may be treated include tumors that are not vascularized, or not yet substantially vascularized, as well as vascularized tumors. The cancers may be comprised of non-solid tumors (such as leukemias and lymphomas) or may be solid tumors. Types of cancers to be treated with the antibodies of the disclosure include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are included.

The compositions of the disclosure may be administered alone, or in combination with one or more therapeutically effective agents or treatments. The other therapeutically effective agent may be conjugated to or incorporated into the composition of the disclosure, or may be administered as a separate composition. The other therapeutically agent or treatment may be administered prior to, during and/or after the administration of the composition of the disclosure.

Other therapeutically effective agents/treatments include surgery, anti-neoplastics (including chemotherapeutic agents and radiation), anti-angiogenesis agents, antibodies to other targets, small molecules, photodynamic therapy, immunotherapy, cytotoxic agents, cytokines, chemokines, growth inhibitory agents, anti-hormonal agents, kinase inhibitors, cardioprotectants, immunostimulatory agents, immunosuppressive agents, agents that promote proliferation of hematological cells, and protein tyrosine kinase (PTK) inhibitors.

A chemotherapeutic agent may be administered as a prodrug. The term “prodrug” refers to a precursor or derivative form of a pharmaceutically active substance that is less cytotoxic to tumor cells compared to the parent drug and is capable of being enzymatically activated or converted into the more active parent form. The prodrugs that may find use with the compositions and methods as disclosed herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the compositions and methods as disclosed herein include but are not limited to any of the aforementioned chemotherapeutic agents.

The administration of the composition of the disclosure with other agents and/or treatments may occur simultaneously, or separately, via the same or different route, at the same or different times.

Doses

Dosage regimens may be adjusted to provide the optimum desired response (e.g., a therapeutic or prophylactic response).

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the disclosure are dictated by and directly dependent on the unique characteristics of the active compound and the particular therapeutic or prophylactic effect to be achieved.

The composition of the disclosure may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Various embodiments of dosage ranges are contemplated by this disclosure. It will be appreciated by the skilled artisan that using information and tools in the art such as pharmacokinetic analysis or allometric scaling allows one to extrapolate animal data to humans. Dosage units for a therapeutic agent such as anti-CTLA-4 or anti-CD137 antibody may be expressed as a flat dose, i.e., 100 mg, 200 mg, 300 mg, or as a patient-specific dose, i.e., mg/kg (mg therapeutic agent/kg of body weight) or mg/m² (quantity in milligrams per square meter of body surface area).

In one embodiment, the dose of the attenuated Listeria strain comprised by the immunogenic composition disclosed herein is administered to a subject at a dose of 1×10⁷-3.31×10¹⁰ CFU. In another embodiment, the dose is 1×10⁸-3.31×10¹⁰ CFU. In another embodiment, the dose is 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-150×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹ CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU.

In another embodiment, the dose is 1×10⁷ organisms. In another embodiment, the dose is 1×10⁸ organisms. In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms. Each dose and range of doses represents a separate embodiment of the present disclosure.

In another embodiment, the mAb is administered beginning at the same day with the vaccine or within about 24 hours of administering the vaccine. In another embodiment, the mAb is administered beginning at the same day with the vaccine or within about 48 hours of administering the vaccine. In another embodiment, the mAb is administered beginning at the same day with the vaccine or within about 72 hours of administering the vaccine. In another embodiment, the mAb is administered beginning at the same day with the vaccine or within about 96 hours of administering the vaccine. In another embodiment, the mAb is administered about 1-2 days after each vaccination. In another embodiment, the mAb is administered about 2-3 days after the vaccination. In another embodiment, the mAb is administered about 3-4 days after each vaccination. In another embodiment, the mAb is administered about 3-5 days after each vaccination. In another embodiment, the mAb is administered about 24-120 hours after the each administration of the vaccine. In another embodiment, the mAb is administered about 24-96 hours after the each administration of the vaccine. In another embodiment, the mAb is administered about 24-72 hours after the each administration of the vaccine. In another embodiment, the mAb is administered about 24-48 hours after the each administration of the vaccine. In another embodiment, the mAb is administered about 48-96 hours after the each administration of the vaccine. In another embodiment, the mAb is administered about 72-96 hours after the each administration of the vaccine. In another embodiment, the first dose of the mAb is administered about 96 hours after the administration of the first dose of the immunogenic composition comprising a recombinant Listeria strain. In another embodiment, the first dose of the mAb is administered about 72 hours after the administration of the first dose of the immunogenic composition comprising a recombinant Listeria strain. In another embodiment, the first dose of the mAb is administered about 48 hours after the administration of the first dose of the immunogenic composition comprising a recombinant Listeria strain. In another embodiment, the first dose of the mAb is administered about 24 hours after the administration of the first dose of the immunogenic composition comprising a recombinant Listeria strain.

In another embodiment, the dose of anti-CTLA-4 blocking antibody is less than 10 mg/kg. In another embodiment, the dose of anti-CTLA-4 blocking antibody is less than 3 mg/kg. In another embodiment, the dose of anti-CTLA-4 blocking antibody is between 10 mg/kg and 0.05 mg/kg. In another embodiment, the dose of anti-CTLA-4 blocking antibody is between 9 mg/kg and 0.05 mg/kg. In another embodiment, the dose of anti-CTLA-4 blocking antibody is between 5 mg/kg and 0.05 mg/kg. In another embodiment, the dose of anti-CTLA-4 blocking antibody is between 2 mg/kg and 0.05 mg/kg. In another embodiment, the dose of anti-CTLA-4 blocking antibody is between 1 mg/kg and 0.05 mg/kg. In another embodiment, the dose of anti-CTLA-4 blocking antibody is between 0.05 mg/kg and 0.05 mg/kg.

In another embodiment, the dose of anti-CD137 agonist antibody is less than 0.3 mg/kg. In another embodiment, the dose of anti-CD137 agonist antibody is less than 0.1 mg/kg. In another embodiment, the dose of anti-CD137 agonist antibody is between 0.3 mg/kg and 0.025 mg/kg. In another embodiment, the dose of anti-CD137 agonist antibody is between 0.1 mg/kg and 0.025 mg/kg. In another embodiment, the dose of anti-CD137 agonist antibody is between 0.09 mg/kg and 0.025 mg/kg. In another embodiment, the dose of anti-CD137 agonist antibody is between 0.09 mg/kg and 0.05 mg/kg. In another embodiment, the dose of anti-CD137 agonist antibody is between 0.1 mg/kg and 10 mg/kg. In another embodiment, the dose of anti-CD137 agonist antibody is between 0.3 mg/kg and 10 mg/kg. In another embodiment, the dose of anti-CD137 agonist antibody is between 0.3 mg/kg and 9 mg/kg. In another embodiment, the dose of anti-CD137 agonist antibody is between 0.1 mg/kg and 5 mg/kg. In another embodiment, the dose of anti-CD137 agonist antibody is between 0.1 mg/kg and 1 mg/kg.

It will be appreciated by the skilled artisan that the term “Boosting” may encompass administering an additional strain or immunogenic composition or recombinant Listeria strain dose or a therapeutic agent such as anti-CTLA-4 or anti-CD137 antibody alone or in combination to a subject. In another embodiment, of methods of the present disclosure, 2 boosts (or a total of 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered.

In another embodiment, a method of present disclosure further comprises the step of boosting the subject with a recombinant Listeria strain or therapeutic agent as disclosed herein. In another embodiment, the recombinant Listeria strain used in the booster inoculation is the same as the strain used in the initial “priming” inoculation. In another embodiment, the booster strain is different from the priming strain. In another embodiment, the same doses are used in the priming and boosting inoculations. In another embodiment, a larger dose is used in the booster. In another embodiment, a smaller dose is used in the booster. In another embodiment, the methods of the present disclosure further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost strain is experimentally determined by the skilled artisan. In another embodiment, the period between a prime and a boost strain is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost strain is administered 8-10 weeks after the prime strain.

In another embodiment, a method of the present disclosure further comprises boosting the subject with a immunogenic composition comprising an attenuated Listeria strain disclosed herein. In another embodiment, a method of the present disclosure comprises the step of administering a booster dose of the immunogenic composition comprising the attenuated Listeria strain disclosed herein. In another embodiment, the booster dose is an alternate form of the immunogenic composition. In another embodiment, the methods of the present disclosure further comprise the step of administering to the subject a booster immunogenic composition. In one embodiment, the booster dose follows a single priming dose of the immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the attenuated Listeria disclosed herein is experimentally determined by the skilled artisan. In another embodiment, the dose is experimentally determined by a skilled artisan. In another embodiment, the period between a prime and a boost dose is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost dose is administered 8-10 weeks after the prime dose of the immunogenic composition.

In another embodiment, a method of present invention further comprises the step of inoculating the human subject with an immunogenic composition comprising the E7 antigen. In another embodiment, the immunogenic composition comprises a recombinant E7 protein or fragment thereof. In another embodiment, the immunogenic composition comprises a nucleotide molecule expressing a recombinant E7 protein or fragment thereof. In another embodiment, the non-Listerial inoculation is administered after the Listerial inoculation. In another embodiment, the non-Listerial inoculation is administered before the Listerial inoculation.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Strain 20:1226-31 (2002); Tanghe, A., Infect. Immun. 69:3041-7 (2001). Providing antigen in different forms in the prime and the boost injections appears to maximize the immune response to the antigen. DNA strain priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Strain 20:1039-45 (2002); Billaut-Mulot, O. et al., Strain 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNAlpoloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appl. Publication No. US 2002/0165172 A1 describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HIV, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

“Administration” to a subject is not limited to any particular delivery system and may include, without limitation, parenteral (including subcutaneous, intravenous, intramedullary, intraarticular, intramuscular, or intraperitoneal injection) rectal, topical, transdermal or oral (for example, in capsules, suspensions or tablets). Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

The composition of the disclosure may be administered parenterally (e.g., intravenous, subcutaneous, intraperitoneal, intramuscular). Further, the composition of the disclosure may be administered by intravenous infusion or injection. The composition of the disclosure may be administered by intramuscular or subcutaneous injection. In some embodiments, the composition of the disclosure may be administered orally. As used herein, a “composition” refers to any composition that contains a pharmaceutically effective amount of one or more active ingredients.

The term “a subject in need thereof” means a person having a cancer. The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis.

The metastatic cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The metastatic cancer may be due to a process such as lymphatic or hematogeneous spread. The metastatic cancer may also be caused by a tumor cell that comes to rest at another site, re-penetrates through the vessel or walls, continues to multiply, and eventually forms another clinically detectable tumor. The metastatic cancer may be this new tumor, which may be a metastatic (or secondary) tumor.

The metastatic cancer may be caused by tumor cells that have metastasized. which may be a secondary or metastatic tumor. The cells of the metastatic tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The metastatic cancer may have an origin from any tissue. The metastatic cancer may originate from melanoma. colon, breast, or prostate, and thus may be made up of cells that were originally skin, colon, breast, or prostate, respectively. The metastatic cancer may also be a hematological malignancy, which may be lymphoma. The metastatic cancer may invade a tissue such as liver, lung, bladder, or intestinal.

The determination of the response of a subject to a specific therapy can be determined using any assessment criterion used in oncology and known by persons skilled in the art. Assessment parameters useful for describing progression of a disease include: disease-free progression which, as used herein, describes the ratio of subjects in complete remission who have not had disease relapse during the time period under study; objective response. which, as used in the present invention, describes the ratio of subjects treated in whom a complete or partial response is observed; tumor control, which, as used in the present invention, relates to the ratio of people treated in whom a complete response, partial response, minor response or stable disease 6 months is observed; progression-free survival which, as used herein, is defined as the time from the beginning of the treatment until the first measurement of cancer growth. In a preferred embodiment, the response of a subject is determined by means of a parameter selected from time to progression and survival. In an exemplary embodiment, a subject's response to a treatment or preventative method provided herein should be statistically significant. The determination of whether a response is statistically significant can be carried out using statistical evaluation tools such as confidence intervals, determination of the p value, Student's t-test. Mann-Whitney test. etc. Preferred confidence intervals are at least 50%. at least 60%. at least 70%, at least 80%, at least 90%, at least 95%. Preferably, p values are 0.2, 0.1, or 0.05.

The term “at risk for metastasis” means a determination made by a physician based on an assessment of disease progression, risk factors, and or prognostic markers that a patient's primary cancer is likely to spread from one organ or part to another non-adjacent organ or part. Examples of methods and prognostic markers useful for identifying patients who are at risk for developing metastases are described in U9133523, U.S. Pat. Nos. 7,482,123, 7,892,740, and Weigelt, B. et al. (August 2005). “Breast cancer metastasis: markers and models.” Nature Reviews Cancer, 5:591-602).

### LISTING OF EMBODIMENTS

The subject matter disclosed herein includes, but is not limited to, the following embodiments.

1. A method of promoting an antigen-specific memory T cell population comprising, administering to a subject an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CTLA-4 antibody or a functional fragment thereof.

2. A method for preventing reoccurrence of a tumor in a subject in need thereof, the method comprising administering to the subject an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CTLA-4 antibody or a functional fragment thereof.

3. A method for treating metastatic cancer in a subject in need thereof, the method comprising administering to the subject an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CTLA-4 antibody or a functional fragment thereof.

4. A method for preventing metastasis in a cancer patient at risk for metastasis, the method comprising administering to the patient an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CTLA-4 antibody or a functional fragment thereof.

5. The method of any one of embodiments 1-4, wherein a first dose of the composition comprising an anti-CTLA-4 antibody or a functional fragment thereof is administered about 72 hours after the administration of a first dose of the immunogenic composition comprising a recombinant Listeria strain.

6. The method of any one of embodiments 1-4, wherein a first dose of the composition comprising an anti-CTLA-4 antibody or a functional fragment thereof is administered about 48 hours after the administration of a first dose of the immunogenic composition comprising a recombinant Listeria strain.

7. The method of any one of embodiments 1-6, wherein the immunogenic composition comprising a recombinant Listeria strain is administered at a dose of about 1×10⁹ CFU.

8. The method of any one of embodiments 1-7, wherein the composition comprising an anti-CTLA-4 antibody or a functional fragment thereof is administered at a dose between about 0.05 mg/kg and about 5 mg/kg.

9. The method of any one of embodiments 1-8, wherein the subject has a progression free survival of at least 3 months.

10. A method of promoting an antigen-specific memory T-cell population comprising, administering to a subject an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CD137 antibody or a functional fragment thereof.

11. A method for preventing reoccurrence of a tumor in a subject in need thereof, the method comprising administering to the subject an effective amount of a combination comprising, i. a immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CD137 antibody or a functional fragment thereof.

12. A method for treating metastatic cancer in a subject in need thereof, the method comprising administering to the subject an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CD137 antibody or a functional fragment thereof.

13. A method for preventing metastasis in a cancer patient at risk for metastasis, the method comprising administering to the patient an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CD137 antibody or a functional fragment thereof.

14. The method of any one of embodiments 10-13, further comprising a step of administering an effective amount of a composition comprising an anti-PD-1 antibody or a functional fragment thereof to the subject.

15. The method of any one of embodiments 10-14, wherein a first dose of the composition comprising an anti-CD137 antibody or a functional fragment thereof is administered about 72 hours after the administration of a first of the immunogenic composition comprising a recombinant Listeria strain.

16. The method of any one of embodiments 10-14, wherein a first dose of the composition comprising an anti-CD137 antibody or a functional fragment thereof is administered about 48 hours after the administration of a first dose the immunogenic composition comprising a recombinant Listeria strain.

17. The method of any one of embodiments 10-16, wherein the immunogenic composition comprising a recombinant Listeria strain is administered at a dose of about 1×10⁹ CFU.

18. The method of any one of embodiments 10-17, wherein the composition comprising an anti-CD137 antibody or a functional fragment thereof is administered at a dose between about 0.1 mg/kg and about 5 mg/kg.

19. The method of any one of embodiments 10-18, wherein the subject has a progression free survival of at least 3 months.

20. The method of any one of embodiments 1-19, wherein the heterologous antigen is a tumor-associated antigen.

21. The method of embodiment 20, wherein the tumor-associated antigen is a human papilloma virus (HPV) E7 antigen.

22. The method of embodiment 21, wherein the tumor-associated antigen is a HPV-16 E7 antigen.

23. The method of any one of embodiments 1-22, wherein the truncated LLO protein comprises SEQ ID NO: 2.

24. The method of any one of embodiments 1-23, wherein the recombinant Listeria strain is a recombinant Listeria monocytogenes strain.

25. The method of any one of embodiments 1-24, wherein the nucleic acid is in an extrachromosomal plasmid in the recombinant Listeria strain.

26. The method of embodiment 25, wherein the plasmid is stably maintained in the recombinant Listeria strain.

27. The method of any one of embodiments 1-26, wherein the Listeria strain comprises a mutation, deletion or inactivation in the endogenous prfA gene.

28. The method embodiment 27, wherein the prfA gene encodes a PrfA protein comprising a D133V mutation.

29. The method of any one of embodiments 1-28, wherein the nucleic acid further comprises a second open reading frame encoding a metabolic enzyme.

30. The method of embodiment 29, wherein the metabolic enzyme complements the mutation, deletion or inactivation.

The following examples are presented in order to more fully illustrate the preferred embodiments of the invention. They should in no way be construed, however, as limiting the broad scope of the invention.

### EXAMPLES

### Example 1: LLO-Antigen Fusions Induce Anti-Tumor Immunity

### Materials and Experimental Methods (Examples 1-2)

### Cell Lines

The C57BL/6 syngeneic TC-1 tumor was immortalized with HPV-16 E6 and E7 and transformed with the c-Ha-ras oncogene. TC-1, disclosed by T. C. Wu (Johns Hopkins University School of Medicine, Baltimore, Md.) is a highly tumorigenic lung epithelial cell expressing low levels of with HPV-16 E6 and E7 and transformed with the c-Ha-ras oncogene. TC-1 was grown in RPMI 1640, 10% FCS, 2 mM L-glutamine, 100 U/ml penicillin, 100 μg/ml streptomycin, 100 μM nonessential amino acids, 1 mM sodium pyruvate, 50 micromolar (mcM) 2-ME, 400 microgram (mcg)/ml G418, and 10% National Collection Type Culture-109 medium at 37⁰ with 10% CO₂. C3 is a mouse embryo cell from C57BL/6 mice immortalized with the complete genome of HPV 16 and transformed with pEJ-ras. EL-4/E7 is the thymoma EL-4 retrovirally transduced with E7.

L. monocytogenes Strains and Propagation

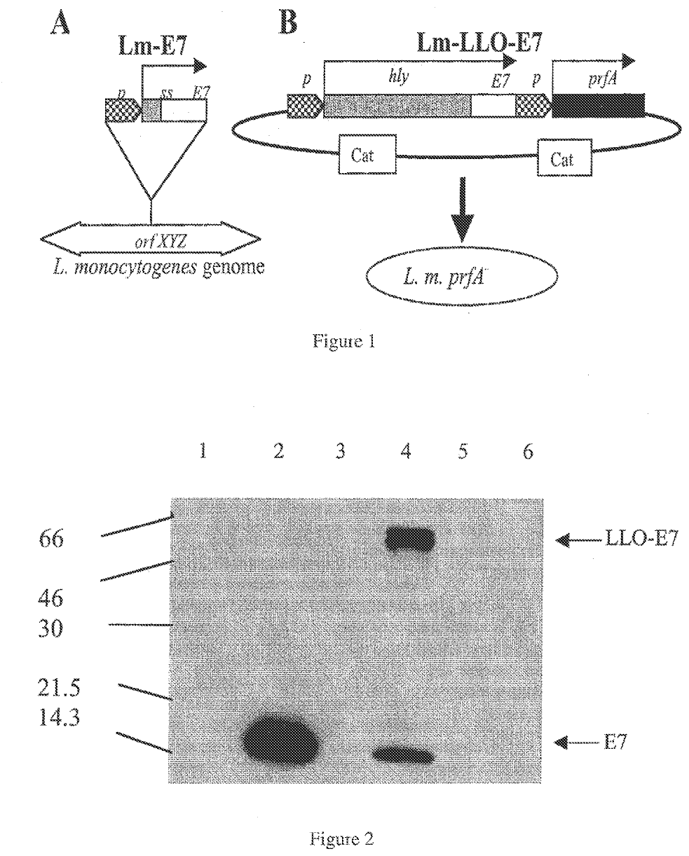

Listeria strains used were Lm-LLO-E7 (hly-E7 fusion gene in an episomal expression system; FIG. 1A), Lm-E7 (single-copy E7 gene cassette integrated into Listeria genome), Lm-LLO-NP (“DP-L2028”; hly-NP fusion gene in an episomal expression system), and Lm-Gag (“ZY-18”; single-copy HIV-1 Gag gene cassette integrated into the chromosome). E7 was amplified by PCR using the primers 5′-GGCTCGAGCATGGAGATACACC-3′ (SEQ ID No: 17; XhoI site is underlined) and 5′-GGGGACTAGTTTATGGTTTCTGAGAACA-3′ (SEQ ID No: 18; SpeI site is underlined) and ligated into pCR2.1 (Invitrogen, San Diego, Calif.). E7 was excised from pCR2.1 by XhoI/SpeI digestion and ligated into pGG-55. The hly-E7 fusion gene and the pluripotential transcription factor PrfA were cloned into pAM401, a multicopy shuttle plasmid (Wirth R et al, J Bacteriol, 165: 831, 1986), generating pGG-55. The hly promoter drives the expression of the first 441 AA of the hly gene product, (lacking the hemolytic C-terminus, referred to below as “ΔLLO,” and having a sequence set forth in SEQ ID Nos: 2-4), which is joined by the XhoI site to the E7 gene, yielding a hly-E7 fusion gene that is transcribed and secreted as LLO-E7. Transformation of a prfA negative strain of Listeria, XFL-7 (disclosed by Dr. Hao Shen, University of Pennsylvania), with pGG-55 selected for the retention of the plasmid in vivo (FIGS. 1A-B). The hly promoter and gene fragment were generated using primers 5′-GGGGGCTAGCCCTCCTTTGATTAGTATATTC-3′ (SEQ ID No: 19; NheI site is underlined) and 5′-CTCCCTCGAGATCATAATTTACTTCATC-3′ (SEQ ID No: 20; XhoI site is underlined). The prfA gene was PCR amplified using primers 5′-GACTACAAGGACGATGACCGACAAGTGATAACCCGGGATCTAAATAAATCCGTTT-3′ (SEQ ID No: 27; XbaI site is underlined) and 5′-CCCGTCGACCAGCTCTTCTTGGTGAAG-3′ (SEQ ID No: 21; SalI site is underlined). Lm-E7 was generated by introducing an expression cassette containing the hly promoter and signal sequence driving the expression and secretion of E7 into the orfZ domain of the LM genome. E7 was amplified by PCR using the primers 5′-GCGGATCCCATGGAGATACACCTAC-3′ (SEQ ID No: 22; BamHI site is underlined) and 5′-GCTCTAGATTATGGTTTCTGAG-3′ (SEQ ID No: 23; XbaI site is underlined). E7 was then ligated into the pZY-21 shuttle vector. LM strain 10403S was transformed with the resulting plasmid, pZY-21-E7, which includes an expression cassette inserted in the middle of a 1.6-kb sequence that corresponds to the orfX, Y, Z domain of the LM genome. The homology domain allows for insertion of the E7 gene cassette into the orfZ domain by homologous recombination. Clones were screened for integration of the E7 gene cassette into the orfZ domain. Bacteria were grown in brain heart infusion medium with (Lm-LLO-E7 and Lm-LLO-NP) or without (Lm-E7 and ZY-18) chloramphenicol (20 μg/ml). Bacteria were frozen in aliquots at −80° C. Expression was verified by Western blotting (FIG. 2).

### Western Blotting

Listeria strains were grown in Luria-Bertoni medium at 37° C. and were harvested at the same optical density measured at 600 nm. The supernatants were TCA precipitated and resuspended in 1× sample buffer supplemented with 0.1 N NaOH. Identical amounts of each cell pellet or each TCA-precipitated supernatant were loaded on 4-20% Tris-glycine SDS-PAGE gels (NOVEX, San Diego, Calif.). The gels were transferred to polyvinylidene difluoride and probed with an anti-E7 monoclonal antibody (mAb) (Zymed Laboratories, South San Francisco, Calif.), then incubated with HRP-conjugated anti-mouse secondary Ab (Amersham Pharmacia Biotech, Little Chalfont, U.K.), developed with Amersham ECL detection reagents, and exposed to Hyperfilm (Amersham Pharmacia Biotech).

### Measurement of Tumor Growth

Tumors were measured every other day with calipers spanning the shortest and longest surface diameters. The mean of these two measurements was plotted as the mean tumor diameter in millimeters against various time points. Mice were sacrificed when the tumor diameter reached 20 mm. Tumor measurements for each time point are shown only for surviving mice.

### Effects of Listeria Recombinants on Established Tumor Growth

Six- to 8-wk-old C57BL/6 mice (Charles River) received 2×10³ TC-1 cells s.c. on the left flank. One week following tumor inoculation, the tumors had reached a palpable size of 4-5 mm in diameter. Groups of eight mice were then treated with 0.1 LD₅₀ i.p. Lm-LLO-E7 (10⁷ CFU), Lm-E7 (10⁶ CFU), Lm-LLO-NP (10⁷ CFU), or Lm-Gag (5×10³ CFU) on days 7 and 14.

### ⁵¹Cr Release Assay

C57BL/6 mice, 6-8 wk old, were immunized i.p. with 0.1LD₅₀ Lm-LLO-E7, Lm-E7, Lm-LLO-NP, or Lm-Gag. Ten days post-immunization, spleens were harvested. Splenocytes were established in culture with irradiated TC-1 cells (100:1, splenocytes:TC-1) as feeder cells; stimulated in vitro for 5 days, then used in a standard ⁵¹Cr release assay, using the following targets: EL-4, EL-4/E7, or EL-4 pulsed with E7 H-2b peptide (RAHYNIVTF). E:T cell ratios, performed in triplicate, were 80:1, 40:1, 20:1, 10:1, 5:1, and 2.5:1. Following a 4-h incubation at 37° C., cells were pelleted, and 50 μl supernatant was removed from each well. Samples were assayed with a Wallac 1450 scintillation counter (Gaithersburg, Md.). The percent specific lysis was determined as [(experimental counts per minute (cpm)-spontaneous cpm)/(total cpm−spontaneous cpm)]×100.

### TC-1-Specific Proliferation

C57BL/6 mice were immunized with 0.1 LD₅₀ and boosted by i.p. injection 20 days later with 1 LD₅₀ Lm-LLO-E7, Lm-E7, Lm-LLO-NP, or Lm-Gag. Six days after boosting, spleens were harvested from immunized and naive mice. Splenocytes were established in culture at 5×10⁵/well in flat-bottom 96-well plates with 2.5×10⁴, 1.25×10⁴, 6×10³, or 3×10³ irradiated TC-1 cells/well as a source of E7 Ag, or without TC-1 cells or with 10 μg/ml Con A. Cells were pulsed 45 h later with 0.5 μCi [³H]thymidine/well. Plates were harvested 18 h later using a Tomtec harvester 96 (Orange, Conn.), and proliferation was assessed with a Wallac 1450 scintillation counter. The change in cpm was calculated as experimental cpm—no Ag cpm.

### Flow Cytometric Analysis

C57BL6 mice were immunized intravenously (i.v.) with 0.1 LD₅₀ Lm-LLO-E7 or Lm-E7 and boosted 30 days later. Three-color flow cytometry for CD8 (53-6.7, PE conjugated), CD62 ligand (CD62L; MEL-14, APC conjugated), and E7 H-2Db tetramer was performed using a FACSCalibur® flow cytometer with CellQuest® software (Becton Dickinson, Mountain View, Calif.). Splenocytes harvested 5 days after the boost were stained at room temperature (rt) with H-2Db tetramers loaded with the E7 peptide (RAHYNIVTF) or a control (HIV-Gag) peptide. Tetramers were used at a 1/200 dilution and were disclosed by Dr. Larry R. Pease (Mayo Clinic, Rochester, Minn.) and by the NIAID Tetramer Core Facility and the NIH AIDS Research and Reference Reagent Program. Tetramer⁺, CD⁸, CD62L^{low} cells were analyzed.

### B16F0-Ova Experiment

24 C57BL/6 mice were inoculated with 5×10³ B16F0-Ova cells. On days 3, 10 and 17, groups of 8 mice were immunized with 0.1 LD₅₀ Lm-OVA (10⁶ cfu), Lm-LLO-OVA (10⁸ cfu) and eight animals were left untreated.

### Statistics

For comparisons of tumor diameters, mean and SD of tumor size for each group were determined, and statistical significance was determined by Student's t test. p≤0.05 was considered significant.

### Results

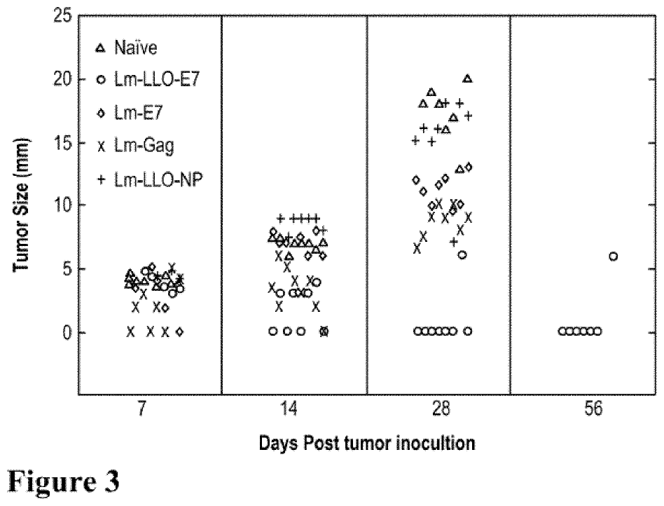

Lm-E7 and Lm-LLO-E7 were compared for their abilities to impact on TC-1 growth. Subcutaneous tumors were established on the left flank of C57BL/6 mice. Seven days later tumors had reached a palpable size (4-5 mm). Mice were vaccinated on days 7 and 14 with 0.1 LD₅₀ Lm-E7, Lm-LLO-E7, or, as controls, Lm-Gag and Lm-LLO-NP. Lm-LLO-E7 induced complete regression of 75% of established TC-1 tumors, while tumor growth was controlled in the other 2 mice in the group (FIG. 3). By contrast, immunization with Lm-E7 and Lm-Gag did not induce tumor regression. This experiment was repeated multiple times, always with very similar results. In addition, similar results were achieved for Lm-LLO-E7 under different immunization protocols. In another experiment, a single immunization was able to cure mice of established 5 mm TC-1 tumors.

In other experiments, similar results were obtained with 2 other E7-expressing tumor cell lines: C3 and EL-4/E7. To confirm the efficacy of vaccination with Lm-LLO-E7, animals that had eliminated their tumors were re-challenged with TC-1 or EL-4/E7 tumor cells on day 60 or day 40, respectively. Animals immunized with Lm-LLO-E7 remained tumor free until termination of the experiment (day 124 in the case of TC-1 and day 54 for EL-4/E7).

Thus, expression of an antigen as a fusion protein with ΔLLO enhances the immunogenicity of the antigen.

### Example 2: Lm-LLO-E7 Treatment Elicits TC-1 Specific Splenocyte Proliferation

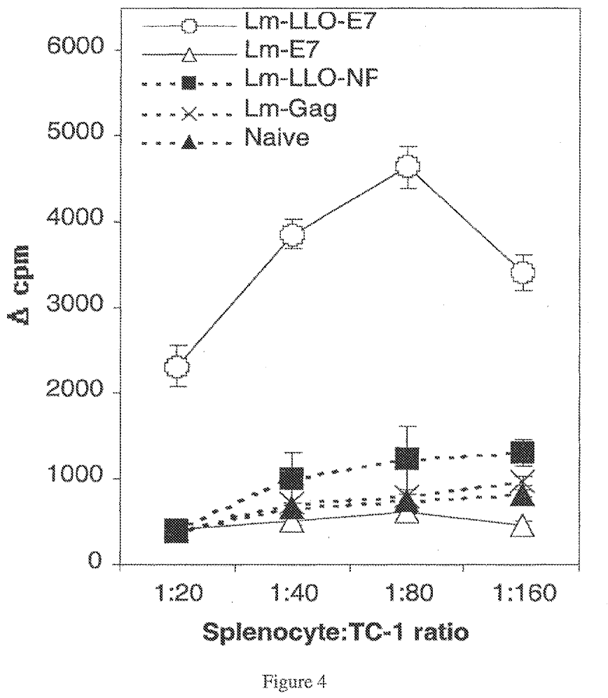

To measure induction of T cells by Lm-E7 with Lm-LLO-E7, TC-1-specific proliferative responses, a measure of antigen-specific immunocompetence, were measured in immunized mice. Splenocytes from Lm-LLO-E7-immunized mice proliferated when exposed to irradiated TC-1 cells as a source of E7, at splenocyte: TC-1 ratios of 20:1, 40:1, 80:1, and 160:1 (FIG. 4). Conversely, splenocytes from Lm-E7 and rLm control-immunized mice exhibited only background levels of proliferation.

### Example 3: Fusion of E7 to LLO, ActA, or a Pest Amino Acid Sequence Enhances E7-Specific Immunity and Generates Tumor-Infiltrating E7-Specific CD8⁺ Cells

### Materials and Experimental Methods

500 mcl (microliter) of MATRIGEL®, comprising 100 mcl of 2×10³ TC-1 tumor cells in phosphate buffered saline (PBS) plus 400 mcl of MATRIGEL® (BD Biosciences, Franklin Lakes, N.J.) were implanted subcutaneously on the left flank of 12 C57BL/6 mice (n=3). Mice were immunized intraperitoneally on day 7, 14 and 21, and spleens and tumors were harvested on day 28. Tumor MATRIGELs were removed from the mice and incubated at 4° C. overnight in tubes containing 2 milliliters (ml) of RP 10 medium on ice. Tumors were minced with forceps, cut into 2 mm blocks, and incubated at 37° C. for 1 hour with 3 ml of enzyme mixture (0.2 mg/ml collagenase-P, 1 mg/ml DNAse-1 in PBS). The tissue suspension was filtered through nylon mesh and washed with 5% fetal bovine serum+0.05% of NaN₃ in PBS for tetramer and IFN-gamma staining.

Splenocytes and tumor cells were incubated with 1 micromole (mcm) E7 peptide for 5 hours in the presence of brefeldin A at 10⁷ cells/ml. Cells were washed twice and incubated in 50 mcl of anti-mouse Fc receptor supernatant (2.4 G2) for 1 hour or overnight at 4° C. Cells were stained for surface molecules CD8 and CD62L, permeabilized, fixed using the permeabilization kit Golgi-Stop® or Golgi-Plug® (Pharmingen, San Diego, Calif.), and stained for IFN-gamma. 500,000 events were acquired using two-laser flow cytometer FACSCalibur and analyzed using Cellquest Software (Becton Dickinson, Franklin Lakes, N.J.). Percentages of IFN-gamma secreting cells within the activated (CD62L^{low}) CD⁸ T cells were calculated.

For tetramer staining, H-2D^{b} tetramer was loaded with phycoerythrin (PE)-conjugated E7 peptide (RAHYNIVTF, SEQ ID NO: 24), stained at rt for 1 hour, and stained with anti-allophycocyanin (APC) conjugated MEL-14 (CD62L) and FITC-conjugated CD80 at 4° C. for 30 min. Cells were analyzed comparing tetramer+CD⁸ CD62L^{low} cells in the spleen and in the tumor.

### Results

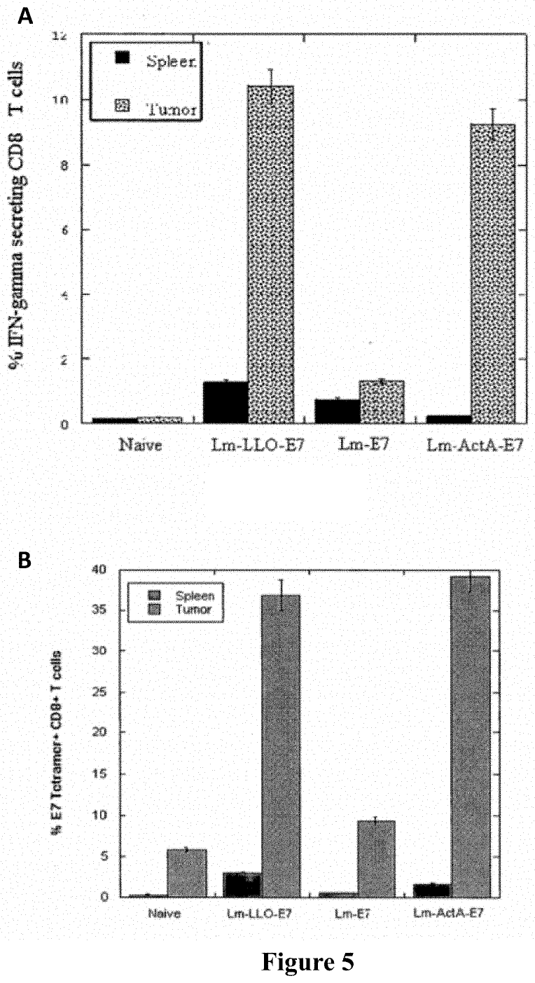

To analyze the ability of Lm-ActA-E7 to enhance antigen specific immunity, mice were implanted with TC-1 tumor cells and immunized with either Lm-LLO-E7 (1×10⁷ CFU), Lm-E7 (1×10⁶ CFU), or Lm-ActA-E7 (2×10⁸ CFU), or were untreated (naïve). Tumors of mice from the Lm-LLO-E7 and Lm-ActA-E7 groups contained a higher percentage of IFN-gamma-secreting CD8⁺ T cells (FIG. 5A) and tetramer-specific CD8⁺ cells (FIG. 5B) than in Lm-E7 or naive mice.

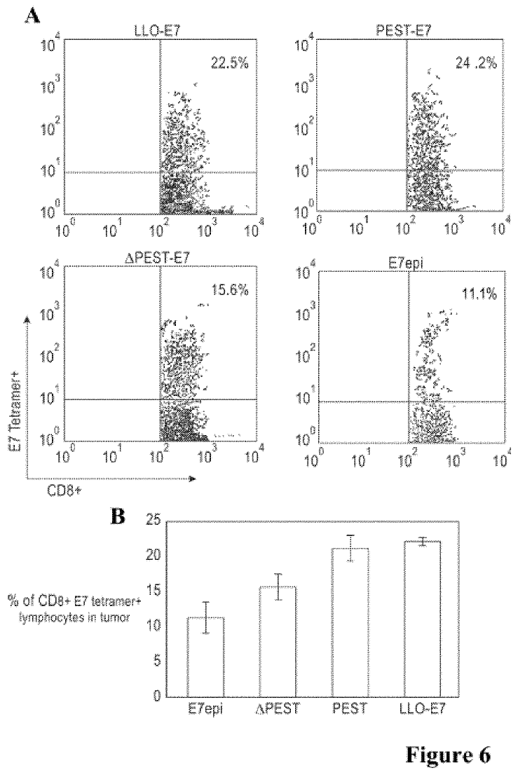

In another experiment, tumor-bearing mice were administered Lm-LLO-E7, Lm-PEST-E7, Lm-ΔPEST-E7, or Lm-E7epi, and levels of E7-specific lymphocytes within the tumor were measured. Mice were treated on days 7 and 14 with 0.1 LD₅₀ of the 4 vaccines. Tumors were harvested on day 21 and stained with antibodies to CD62L, CD8, and with the E7/Db tetramer. An increased percentage of tetramer-positive lymphocytes within the tumor were seen in mice vaccinated with Lm-LLO-E7 and Lm-PEST-E7 (FIG. 6A). This result was reproducible over three experiments (FIG. 6B).

Thus, Lm-LLO-E7, Lm-ActA-E7, and Lm-PEST-E7 are each efficacious at induction of tumor-infiltrating CD8⁺ T cells and tumor regression.

### Example 4: Passaging of Listeria Vaccine Vectors Through Mice Elicits Increased Immune Responses to Heterologous and Endogenous Antigens

### Materials and Experimental Methods

### Bacterial Strains

L. monocytogenes strain 10403S, serotype 1 (ATCC, Manassas, Va.) was the wild type organism used in these studies and the parental strain of the constructs described below. Strain 10403S has an LD₅₀ of approximately 5×10⁴ CFU when injected intraperitoneally into BALB/c mice. “Lm-Gag” is a recombinant LM strain containing a copy of the HIV-1 strain HXB (subtype B laboratory strain with a syncytia-forming phenotype) gag gene stably integrated into the Listerial chromosome using a modified shuttle vector pKSV7. Gag protein was expressed and secreted by the strain, as determined by Western blot. All strains were grown in brain-heart infusion (BHI) broth or agar plates (Difco Labs, Detroit, Mich.).

### Bacterial Culture

Bacteria from a single clone expressing the passenger antigen and/or fusion protein were selected and cultured in BHI broth overnight. Aliquots of this culture were frozen at 70° C. with no additives. From this stock, cultures were grown to 0.1-0.2 O.D. at 600 nm, and aliquots were again frozen at −70° C. with no additives. To prepare cloned bacterial pools, the above procedure was used, but after each passage a number of bacterial clones were selected and checked for expression of the target antigen, as described herein. Clones in which expression of the foreign antigen was confirmed were used for the next passage.

### Passage of Bacteria in Mice

6-8 week old female BALB/c (H-2d) mice were purchased from Jackson Laboratories (Bar Harbor, Me.) and were maintained in a pathogen-free microisolator environment. The titer of viable bacteria in an aliquot of stock culture, stored frozen at −70 OC, was determined by plating on BHI agar plates on thawing and prior to use. In all, 5×10³ bacteria were injected intravenously into BALB/c mice. After 3 days, spleens were harvested, homogenized, and serial dilutions of the spleen homogenate were incubated in BHI broth overnight and plated on BHI agar plates. For further passage, aliquots were again grown to 0.1-0.2 O.D., frozen at −70° C., and bacterial titer was again determined by serial dilution. After the initial passage (passage 0), this sequence was repeated for a total of 4 times.

### Intracellular Cytokine Stain for IFN-Gamma

Lymphocytes were cultured for 5 hours in complete RPMI-10 medium supplemented with 50 U/ml human recombinant IL-2 and 1 microliter/ml Brefeldin A (Golgistop™; PharMingen, San Diego, Calif.) in the presence or absence of either the cytotoxic T-cell (CTL) epitope for HIV-GAG (AMQMLKETI; SEQ ID No: 25), Listeria LLO (GYKDGNEYI; SEQ ID No: 26) or the HPV virus gene E7 (RAHYNIVTF (SEQ ID No: 24), at a concentration of 1 micromole. Cells were first surface-stained, then washed and subjected to intracellular cytokine stain using the Cytofix/Cytoperm kit in accordance with the manufacturer's recommendations (PharMingen, San Diego, Calif.). For intracellular IFN-gamma stain, FITC-conjugated rat anti-mouse IFN-gamma monoclonal antibody (clone XMG 1.2) and its isotype control Ab (rat IgG1; both from PharMingen) was used. In all, 10⁶ cells were stained in PBS containing 1% Bovine Serum Albumin and 0.02% sodium azide (FACS Buffer) for 30 minutes at 4° C. followed by 3 washes in FACS buffer. Sample data were acquired on either a FACScan™ flowcytometer or FACSCalibur™ instrument (Becton Dickinson, San Jose, Calif.). Three-color flow cytometry for CD8 (PERCP conjugated, rat anti-mouse, clone 53-6.7 Pharmingen, San Diego, Calif.), CD62L (APC conjugated, rat anti-mouse, clone MEL-14), and intracellular IFN-gamma was performed using a FACSCalibur™ flow cytometer, and data were further analyzed with CELLQuest software (Becton Dickinson, Mountain View, Calif.). Cells were gated on CD8 high and CD62L^{low} before they were analyzed for CD8+ and intracellular IFN-gamma staining.

### Results

### Passaging in Mice Increases the Virulence of Recombinant Listeria Monocytogenes

Three different constructs were used to determine the impact of passaging on recombinant Listeria vaccine vectors. Two of these constructs carry a genomic insertion of the passenger antigen: the first comprises the HIV gag gene (Lm-Gag), and the second comprises the HPV E7 gene (Lm-E7). The third (Lm-LLO-E7) comprises a plasmid with the fusion gene for the passenger antigen (HPV E7) fused with a truncated version of LLO and a gene encoding PrfA, the positive regulatory factor that controls Listeria virulence factors. This plasmid was used to complement a prfA negative mutant so that in a live host, selection pressures would favor conservation of the plasmid, because without it the bacterium is avirulent. All 3 constructs had been propagated extensively in vitro for many bacterial generations.

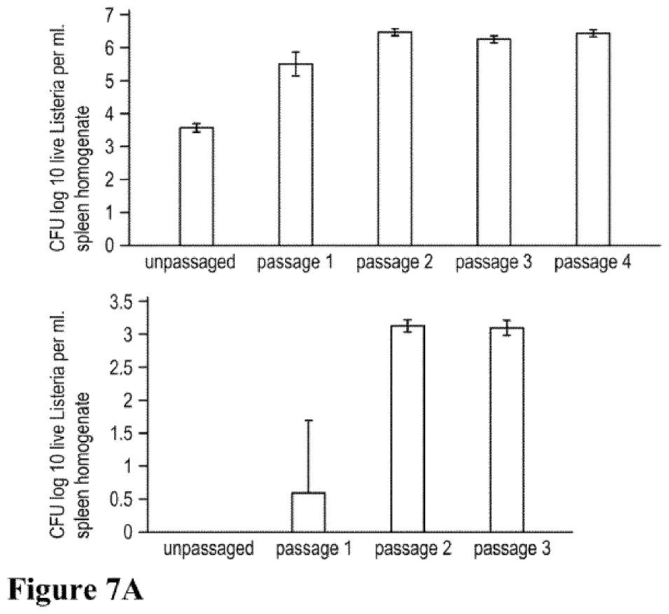

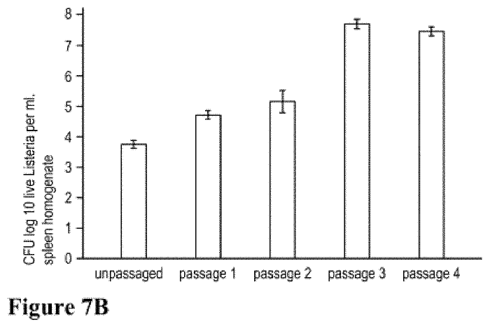

Passaging the bacteria resulted in an increase in bacterial virulence, as measured by numbers of surviving bacteria in the spleen, with each of the first 2 passages. For Lm-Gag and Lm-LLO-E7, virulence increased with each passage up to passage 2 (FIG. 7A). The plasmid-containing construct, Lm-LLO-E7, demonstrated the most dramatic increase in virulence. Prior to passage, the initial immunizing dose of Lm-LLO-E7 had to be increased to 10⁷ bacteria and the spleen had to be harvested on day 2 in order to recover bacteria (whereas an initial dose of 10³ bacteria for Lm-Gag was harvested on day 3). After the initial passage, the standard dosage of Lm-LLO-E7 was sufficient to allow harvesting on day 3. For Lm-E7, virulence increased by 1.5 orders of magnitude over unpassaged bacteria (FIG. 7B).

Thus, passage through mice increases the virulence of Listeria vaccine strains.

Passaging Increases the Ability of L. monocytogenes to Induce CD8⁺ T Cells

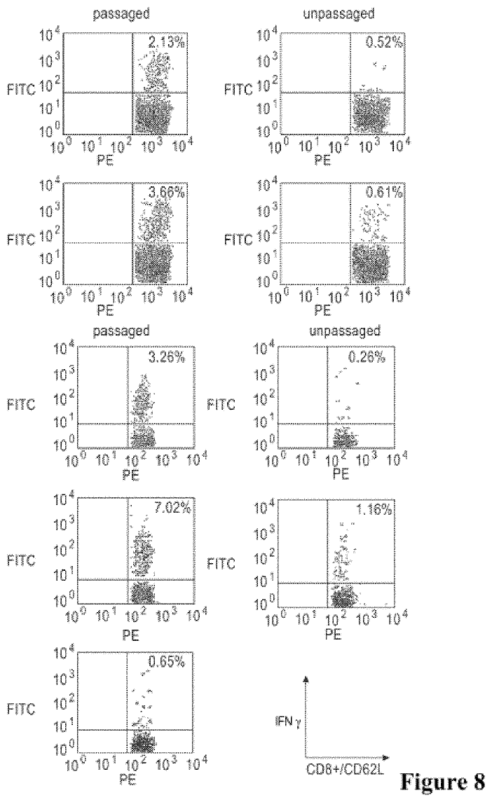

Next, the effect of passaging on induction of antigen-specific CD8⁺ T cells was determined by intracellular cytokine staining with immunodominant peptides specific for MHC-class I using HIV-Gag peptide AMQMLKETI (SEQ ID No: 25) and LLO 91-99 (GYKDGNEYI; SEQ ID No: 26). Injection of 10³ CFU passaged bacteria (Lm-Gag) into mice elicited significant numbers of HIV-Gag-specific CD8⁺ T cells, while the same dose of non-passaged Lm-Gag induced no detectable Gag-specific CD8⁺ T cells. Even increasing the dose of unpassaged bacteria 100-fold did not compensate for their relative avirulence; in fact, no detectable Gag-specific CD8⁺ T cells were elicited even at the higher dose. The same dose increase with passaged bacteria increased Gag-specific T cell induction by 50% (FIG. 8). The same pattern of induction of antigen-specific CD8⁺ T cells was observed with LLO-specific CD8⁺ T cells, showing that these results were not caused by the properties of the passenger antigen, since they were observed with LLO, an endogenous Listeria antigen.

Thus, passage through mice increases the immunogenicity of Listeria vaccine strains.

### Example 5: A PrfA-Containing Plasmid is Stable in an LM Strain with a PrfA Deletion in the Absence of Antibiotics

### Materials and Experimental Methods

### Bacteria

L. monocytogenes strain XFL7 contains a 300 base pair deletion in the prfA gene XFL7 carries pGG55 which partially restores virulence and confers CAP resistance, and is described in United States Patent Application Publication No. 200500118184.

Development of Protocol for Plasmid Extraction from Listeria

1 mL of Listeria monocytogenes Lm-LLO-E7 research working cell bank vial was inoculated into 27 mL BH1 medium containing 34 μg/mL CAP and grown for 24 hours at 37° C. and 200 rpm.

Seven 2.5 mL samples of the culture were pelleted (15000 rpm for 5 minutes), and pellets were incubated at 37° C. with 50 μl lysozyme solution for varying amounts of time, from 0-60 minutes.

Lysozyme solution:
29 μl 1 M dibasic Potassium Phosphate
21 μl 1 M monobasic Potassium Phosphate
500 μl 40% Sucrose (filter sterilized through 0.45/μm filter)
450 μl water
60 μl lysozyme (50 mg/mL)

After incubation with the lysozyme, the suspensions were centrifuged as before and the supernatants discarded. Each pellet was then subjected to plasmid extraction by a modified version of the QIAprep Spin Miniprep Kit® (Qiagen, Germantown, Md.) protocol. The changes to the protocol were as follows:
1. The volumes of buffers PI, P2 and N3 were all increased threefold to allow complete lysis of the increased biomass.
2. 2 mg/mL of lysozyme was added to the resuspended cells before the addition of P2. The lysis solution was then incubated at 37° C. for 15 minutes before neutralization.
3. The plasmid DNA was resuspended in 30 μL rather than 50 μL to increase the concentration.

In other experiments, the cells were incubated for 15 min in P1 buffer+Lysozyme, then incubated with P2 (lysis buffer) and P3 (neutraliztion buffer) at room temperature.

Equal volumes of the isolated plasmid DNA from each subculture were run on an 0.8% agarose gel stained with ethidium bromide and visualized for any signs of structural or segregation instability.

The results showed that plasmid extraction from L. monocytogenes Lm-LLO-E7 increases in efficiency with increasing incubation time with lysozyme, up to an optimum level at approximately 50 minutes incubation.

These results provide an effective method for plasmid extraction from Listeria vaccine strains.

### Replica Plating

Dilutions of the original culture were plated onto plates containing LB or TB agar in the absence or presence of 34 μg/mL CAP. The differences between the counts on selective and non-selective agar were used to determine whether there was any gross segregational instability of the plasmid.

### Results

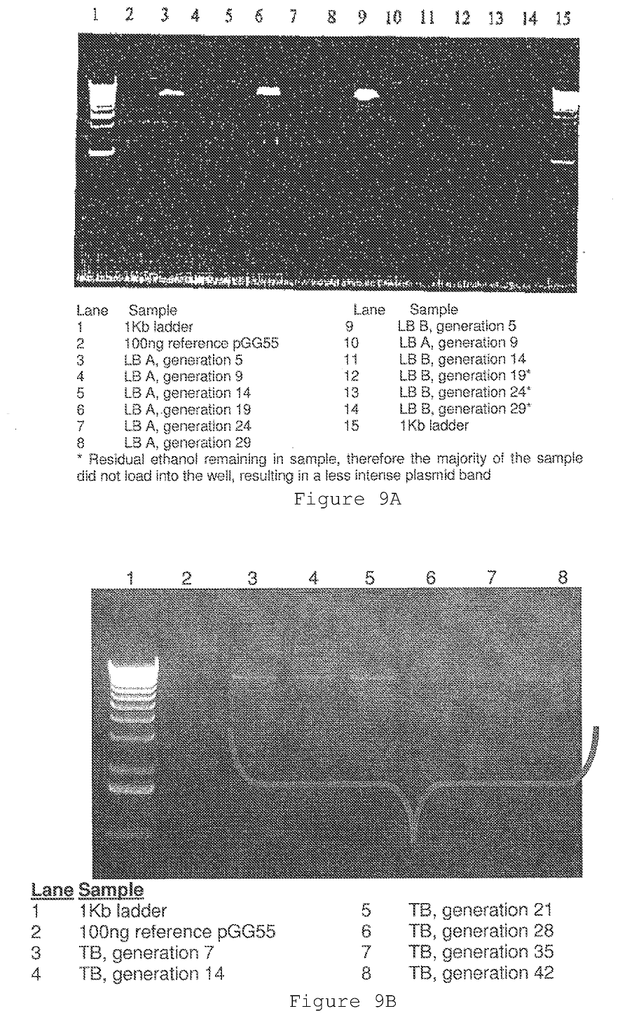

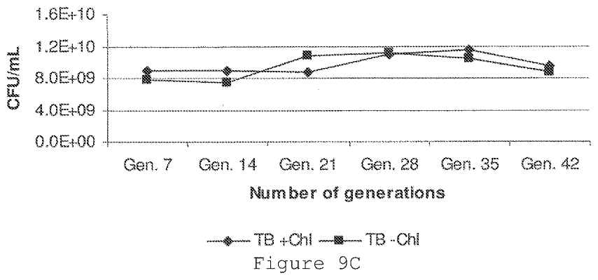

The genetic stability (i.e. the extent to which the plasmid is retained by or remains stably associated with the bacteria in the absence of selection pressure; e.g. antibiotic selection pressure) of the pGG55 plasmid in L. monocytogenes strain XFL7 in the absence of antibiotic was assessed by serial sub-culture in both Luria-Bertani media (LB: 5 g/L NaC, 10 g/ml soy peptone, 5 g/L yeast extract) and Terrific Broth media (TB: 10 g/L glucose, 11.8 g/L soy peptone, 23.6 g/L yeast extract, 2.2 g/L KH₂PO₄, 9.4 g/L K₂HPO₄), in duplicate cultures. 50 mL of fresh media in a 250 mL baffled shake flask was inoculated with a fixed number of cells (1 ODmL), which was then subcultured at 24 hour intervals. Cultures were incubated in an orbital shaker at 37° C. and 200 rpm. At each subculture the OD₆₀₀ was measured and used to calculate the cell doubling time (or generation) elapsed, until 30 generations were reached in LB and 42 in TB. A known number of cells (15 ODmL) at each subculture stage (approximately every 4 generations) were pelleted by centrifugation, and the plasmid DNA was extracted using the Qiagen QIAprep Spin Miniprep® protocol described above. After purification, plasmid DNA was subjected to agarose gel electrophoresis, followed by ethidium bromide staining. While the amount of plasmid in the preps varied slightly between samples, the overall trend was a constant amount of plasmid with respect to the generational number of the bacteria (FIGS. 9A-B). Thus, pGG55 exhibited stability in strain XFL7, even in the absence of antibiotic.

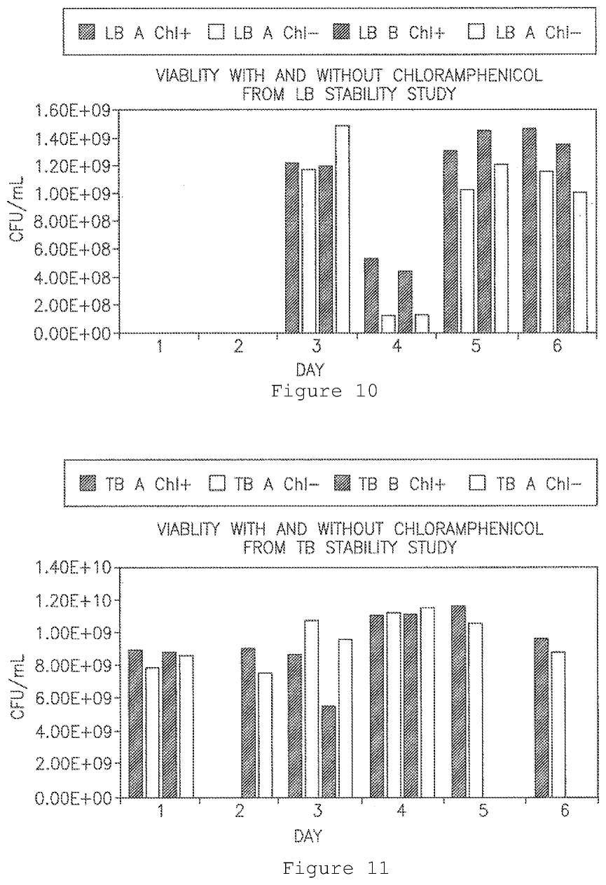

Plasmid stability was also monitored during the stability study by replica plating on agar plates at each stage of the subculture. Consistent with the results from the agarose gel electrophoresis, there was no overall change in the number of plasmid-containing cells throughout the study in either LB or TB liquid culture (FIGS. 10 and 11, respectively).

These findings demonstrate that PrfA-encoding plasmids exhibit stability in the absence of antibiotic in Listeria strains containing mutations in prfA.

### Materials and Methods (Examples 6-10)

PCR Reagents:

The primers used for amplification of the prfA gene and discrimination of the D133V mutation are shown in Table 1. Stock solutions of the primers ADV451, 452 and 453 were prepared by diluting the primers in TE buffer to 400 μM. An aliquot of the stock solution was further diluted to 20 μM in water (PCR grade) to prepare a working solution. Primers were stored at −20° C. The reagents used in the PCR are shown in Table 2.

TABLE 1
Primers ADV451, 452 and 453.

| Primer | Orientation | Sequence (5′→ 3′) | Specificity |
|---|---|---|---|
| ADV451 | Forward | CCTAGCTAAATTTAATGT | D133V |
| | | (SEQ ID NO: 28) | mutation |
| ADV452 | Forward | CCTAGCTAAATTTAATGA | Wild-type |
| | | (SEQ ID NO: 29) | sequence |
| ADV453 | Reverse | TAATTTTCCCCAAGTAGCAGG | Shared |
| | | (SEQ ID NO: 30) | sequence |

TABLE 2
PCR reagents.

| | | | Catalog |
|---|---|---|---|
| | Description | Provider | number |
| 1 | 0.2 ml thin-walled PCR tubes: GeneAmp | Applied | N801- |
| | autoclaved reaction tube with cap | Biosystems | 0612 |
| 2 | Water (PCR reagent) | Sigma | W1754 |
| 3 | Taq DNA Polymerase with 10× reaction | Sigma | D1806 |
| | buffer containing 15 mM MgCl₂ | | |
| 4 | Set of deoxynucleotides (dNTPs), 10 mM | Sigma | D7295 |
| | each | | |
| 5 | Primers ADV451, ADV452 and ADV453 | Invitrogen | |
| 6 | Template DNA, midipreparations of pGG55 | | |
| | Plasmids | | |
| 7 | Thermal cycler PTC200 (48 wells block) | MJ Research | |

### Plasmid DNA Preparation

pGG55 plasmids with (pGG55 D133V) and without (pGG55 WT) the prfA mutation were extracted and purified by midipreparations either from E. coli or Listeria monocytogenes using the PureLink™ HiPure Plasmid Midiprep Kit (Invitrogen, K2100-05), according to the manufacturer's instructions. For plasmid purification from Listeria, bacterial strains carrying the pGG55 D133V or WT plasmids were streak plated from frozen stocks in BHI agar plates supplemented with chloramphenicol (25 μg/ml). A single colony from each strain was grown in 5 ml of selective medium (BHI broth with 25 μg/ml of chloramphenicol) for 6 hours with vigorous shaking at 37° C. and subinoculated 1:500 in 100 ml of selective medium for overnight growth under similar conditions. Bacteria from the overnight culture were harvested by centrifugation at 4,000×g for 10 minutes and resuspended buffer R3 (resuspension buffer) containing 2 mg/ml of lysozyme (Sigma, L7001). The bacteria suspension was incubated for at least 1 hour at 37° C. before proceeding to the regular protocol. Concentration and purity of the eluted plasmids were measured in a spectrophotometer at 260 nm and 280 nm. To prepare the template DNAs, the pGG55 D133V and WT plasmids were resuspended in water to a final concentration of 1 ng/μl from the midiprep stock solution. For the pGG55 WT plasmid, serial 10-fold dilutions from the 1 ng/μl solution were prepared, corresponding to dilutions from 10⁻¹ to 10⁻⁷.

prfA Specific PCR Protocol to Test Clinical Grade Material

The reaction mixture contained 1×PCR buffer, 1.5 mM MgCl₂, 0.8 mM dNTPs, 0.4 μM of each primer, 0.05 U/μl of Taq DNA polymerase and 0.04 ng/μl of the pGG55 D133V template plasmid. For each test, 10 tubes were required and the key components in each tube in a 25 μl reaction are shown in the Table 3. For the PCR reaction, a master mix was prepared with enough reagents for 11 reactions as shown in Table 4, and 24 μl of this PCR mix was added to each tube. Subsequently, a total of 1 μl of the serially diluted pGG55 WT plasmid was added to the corresponding tubes: 1 ng in tube 3; 100 μg in tube 4; 10 μg in tube 5; 1 μg in tube 6; 100 fg in tube 7; 10 fg in tube 8; 1 fg in tube 9; 0.1 fg in tube 10. This serial dilution was used to calibrate a standard curve to determine the method sensitivity. Additionally, 0.5 μl of water and 0.5 μl of primer ADV451 (20 μM stock) were added in tube 1, and 1 μl of water added in tube 2, completing 25 μl of final volume. The quantities of each reagent per tube for a 25 μl reaction are shown in Table 5. The PCR cycling conditions used in the reaction are shown in Table 6.

After conclusion of the PCR reaction, 5 μl of gel-loading buffer (6×, with bromophenol blue) was added to each sample and 10 μl were analyzed by electrophoresis in 1.2% agarose gel in TBE buffer. The gel dimensions were 7 cm×7 cm×1 cm with a 15 sample wells (1 mm×2 mm) comb. The gel was run at 100 V for −30 minutes, until the bromophenol blue dye reached the middle of the gel. The gel was stained in ethidium bromide (0.5 μg/ml) for 20 minutes, destaining in water for 10 minutes. The gel is visualized by illumination with UV light and photographed. The image was analyzed using a band densitometry software (Quantity One version 4.5.1, BioRad).

TABLE 3
Set of individual PCR reactions to validate the method to detect the
presence of wild-type prfA sequence in Lm-LLO-E7 samples.

| | | | | | Expected |
|---|---|---|---|---|---|
| Tube | Primer A | Primer B | Template DNA | Function | result |
| 1 | ADV451 | ADV453 | 1 ng of pGG55 | Positive control for | Positive |
| | | | (D133V) | the ADV451 reaction | |
| 2 | ADV452 | ADV453 | 1 ng of pGG55 | Negative control for | Negative |
| | | | (D133V) | the ADV452 reaction | |
| | | | | (specificity) | |
| 3 | ADV452 | ADV453 | 1 ng of pGG55 | Positive control for | Positive |
| | | | (wild-type) + 1 ng | the ADV452 reaction | |
| | | | of pGG55 (D133V) | | |
| 4 | ADV452 | ADV453 | 100 pg of pGG55 | Test the sensitivity of | Positive |
| | | | (wild-type) + 1 ng | the reaction | |
| | | | of pGG55 (D133V) | | |
| 5 | ADV452 | ADV453 | 10 pg of pGG55 | Test the sensitivity of | Positive |
| | | | (wild-type) + 1 ng | the reaction | |
| | | | of pGG55 (D133V) | | |
| 6 | ADV452 | ADV453 | 1 pg of pGG55 | Test the sensitivity of | Positive |
| | | | (wild-type) + 1 ng | the reaction | |
| | | | of pGG55 (D133V) | | |
| 7 | ADV452 | ADV453 | 100 fg of pGG55 | Test the sensitivity of | Positive |
| | | | (wild-type) + 1 ng | the reaction | |
| | | | pGG55 (D133V) | | |
| 8 | ADV452 | ADV453 | 10 fg of pGG55 | Test the sensitivity of | Positive |
| | | | (wild-type) + | the reaction | |
| | | | pGG55 (D133V) | | |
| 9 | ADV452 | ADV453 | 1 fg of pGG55 | Test the sensitivity of | Weakly |
| | | | (wild-type) + | the reaction | positive |
| | | | pGG55 (D133V) | | |
| 10 | ADV452 | ADV453 | 0.1 fg of pGG55 | Test the sensitivity of | To be |
| | | | (wild-type) + | the reaction | determined |
| | | | pGG55 (D133V) | | |

TABLE 4
Master PCR mix preparation.

| Reagent | Quantity (μl) |
|---|---|
| Water | 206.25 |
| Taq DNA Polymerase 10x reaction buffer | 27.5 |
| containing 15 mM MgCl₂ | |
| Deoxynucleotides (dNTPs) 10 mM each | 5.5 |
| Primers ADV452 (20 μM in water) | 5.5 |
| Primers ADV453 (20 μM in water) | 5.5 |
| pGG55 D133V (Lm-LLO-E7) plasmid (1 ng/μl) | 11 |
| Taq DNA Polymerase (5 U/μl) | 2.75 |
| Total | 264 |

TABLE 5
PCR protocol for validation of the method to detect the presence of
wild-type prfA sequence using primers ADV451, 452 and 453.

| Reagent | PCR |
|---|---|
| Water | 18.75 μl |
| PCR Buffer 10x + MgCl₂ 15 mM | 2.5 μl |
| Deoxynucleotides mix (dATP, dCTP, dGTP and dTTP) | 0.5 μl |
| 10 mM each | |
| Primer ADV452 (20 μM) | 0.5 μl |
| Primer ADV453 (20 μM) | 0.5 μl |
| Taq DNA polymerase (5 U/μl) | 0.25 μl |
| Template DNA (1 ng/μl) pGG55 D133V | 1 μl |
| Template DNA pGG55 WT (tubes 3 to 10)^{a} | 1 μl |
| Final volume per tube^{b} | 25 μl |

^{a}pGG55 WT (1 ng in tube 3; 100 pg in tube 4; 10 pg in tube 5; 1 pg in tube 6; 100 fg in tube 7; 10 fg in tube 8; 1 fg in tube 9; 0.1 fg in tube 10).
^{b}In tube 1, add 0.5 μl of water and 0.5 μl of primer ADV451 (20 μM stock); in tube 2 add 1 μl of water.

TABLE 6
PCR cycling conditions to detect the presence of wild-type prfA
sequence using primers ADV451, 452 and 453.

| Step | Temperature | Time | Number of cycles |
|---|---|---|---|
| 1. | 94° C. | 2 minutes and 30 seconds | 1 |
| 2. | 94° C. | 30 seconds | 1 |
| 3. | 53° C. | 30 seconds | 1 |
| 4. | 72° C. | 30 seconds | 1 |
| 5. | Repeat steps 2 to 4 | | 12 |
| 6. | 94° C. | 30 seconds | 1 |
| 7. | 50° C. | 30 seconds | 1 |
| 8. | 72° C. | 30 seconds | 1 |
| 9. | Repeat steps 6 to 8 | | 23 |
| 10. | 72° C. | 10 minutes | 1 |

### Sequencing:

Sequencing of the plasmids was done using the dideoxy sequencing method. The plasmids pGG55 D133V and pGG55 WT were mixed at different ratios (1:1, 1:10, 1:100, 1:1,000 and 1:10,000). The total amount of plasmid in the mixture was kept constant (500 gig) and the plasmid containing the wild-type sequence was 10-fold serially diluted in relation to the D133V plasmid to determine the sensitivity of the method.

### Results

### Example 6: Sequencing is not a Sensitive Method to Detect the Reversion of the D133V Mutation

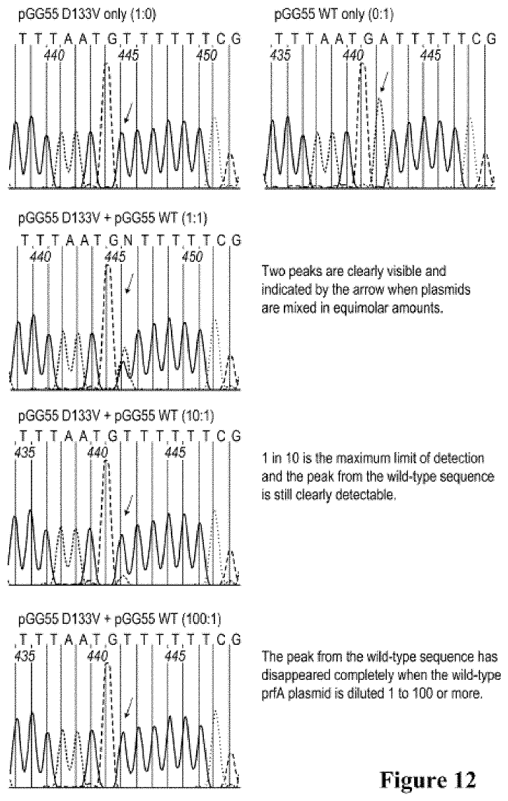

To estimate the sensitivity of sequencing in detecting the wild-type prfA sequence, the pGG55 D133V and WT plasmids were mixed at the different ratios and sequenced. The results are shown in FIG. 12 and reveal that sequencing has a high specificity in discriminating the prfA D133V mutation (FIG. 12). On the other hand, the sensitivity is low and the maximum dilution of wild-type prfA pGG55 plasmid with a detectable peak in the sequence was 1 in 10 (FIG. 12). In conclusion, although sequencing is very specific, the sensitivity of the method is low and not appropriate to screen for the presence of rare events such as revertants of the prfA D133V mutation in Lm-LLO-E7 samples.

### Example 7: Development of a Highly Specific and Sensitive PCR Method to Detect Reversion of the D133V Mutation

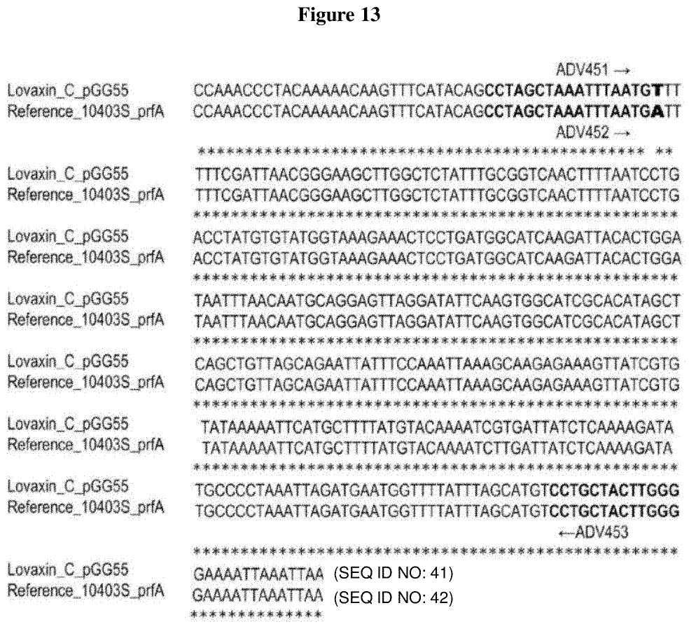

Given the low sensitivity of sequencing to detect rare events, it became imperative to develop a more sensitive method with similar specificity to detect reversion of the D133V mutation to wild-type. To achieve this goal, we designed a PCR-based method that specifically amplifies the wild-type sequence and is sensitive enough to detect at least 1 wild-type copy of prfA in 10,000,000 copies of the D133V mutated sequence. We designed 3 primers for this method: ADV451, ADV452 and ADV453 (Table 1). Both ADV451 and ADV452 are forward primers and differ in the last nucleotide at the 3′ position to discriminate the A-T (D133V) mutation at position 398 of the prfA gene. The ADV453 primer is the reverse primer located approximately 300 bp downstream the annealing site of the ADV451 and ADV452 primers (FIG. 13). The expected PCR band obtained with the primers ADV451 or ADV452 and ADV453 is 326 bp. Under stringent conditions, the ADV451 primer should only amplify the pGG55 D133V plasmid, whereas the ADV452 would be specific to the wild-type prfA sequence.

### Example 8: Specificity of the PCR Method

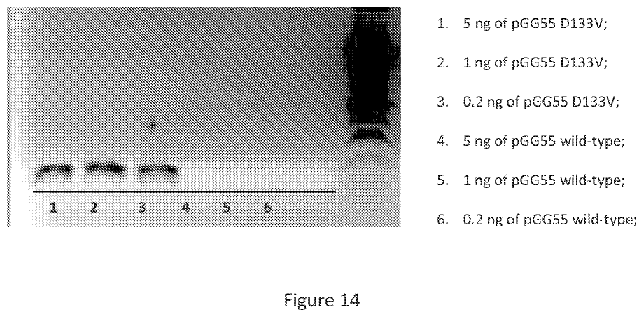

The reaction using the primer ADV451 was very specific and amplified the mutated D133V prfA sequence (lanes 1 to 3), but not the wild-type sequence (lanes 4 to 6). However, a very faint band can be detected in lane 4, when 5 ng of template DNA was used, but not with 1 ng (FIG. 14).

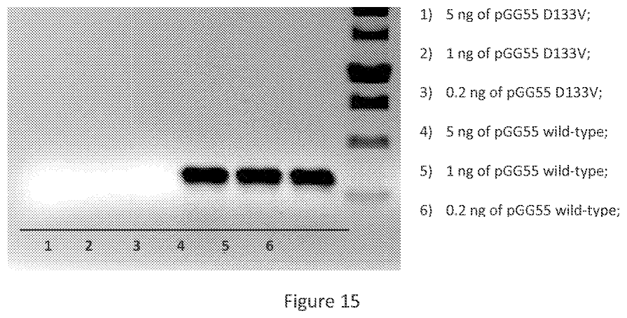

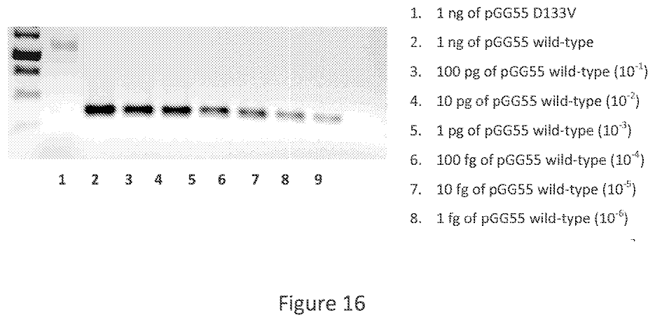

As shown in FIG. 15, the reaction with the ADV452 primer only amplified the wild-type prfA sequence (lanes 4, 5 and 6), and no bands were detected when the pGG55 carrying the D133V PrfA mutation was used as a template (lanes 1, 2 and 3), even when using 5 ng of plasmid in the reaction (FIG. 16). In conclusion, the PCR reactions with primers ADV451 and ADV452 are very specific and able to discriminate the A↔T (D133V) mutation at position 398 of the prfA gene in the pGG55 plasmid. Based on these results, we selected the amount of 1 ng as the standard amount of template DNA to be used in the reaction.

### Example 9: Sensitivity of the PCR Method

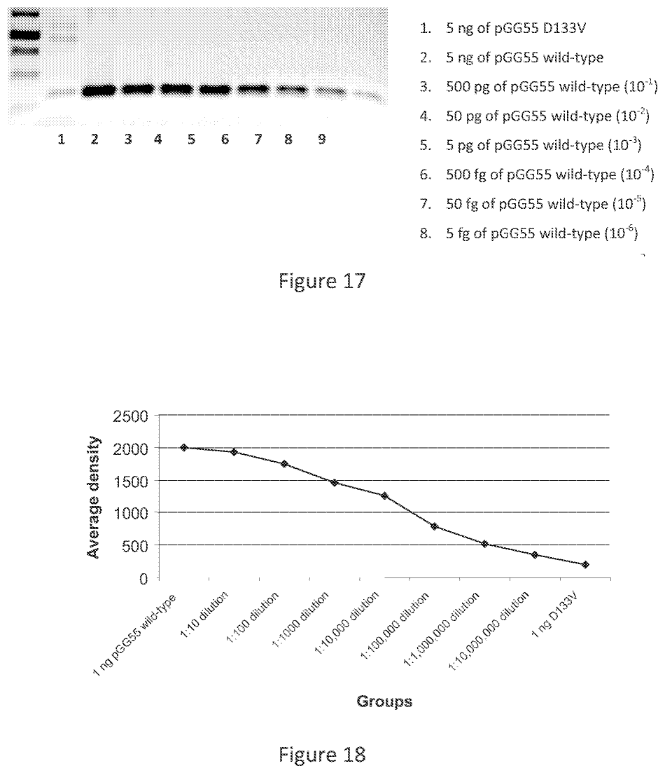

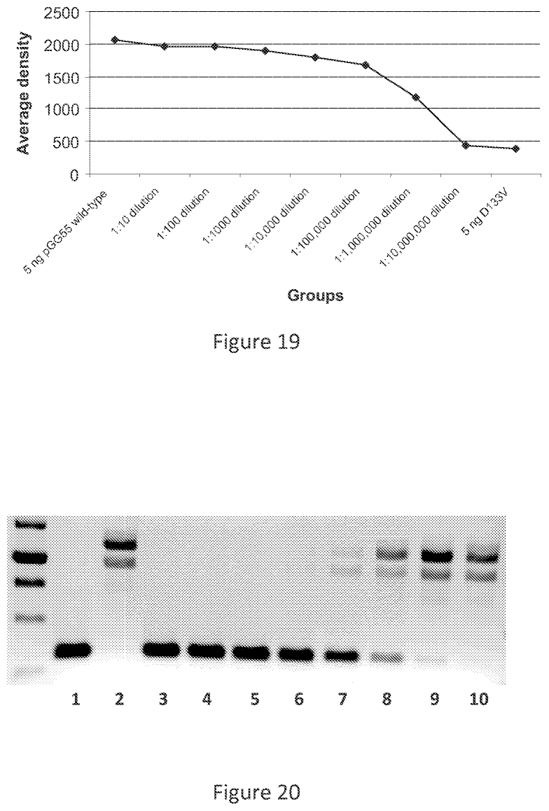

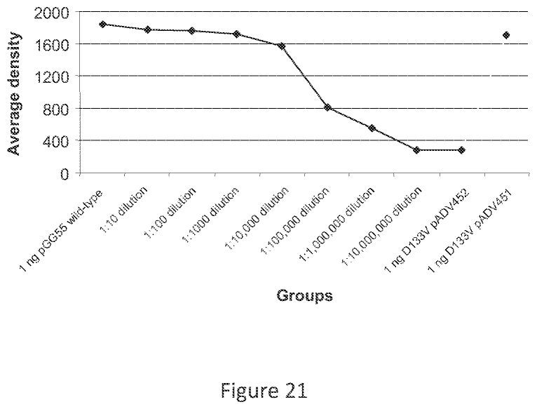

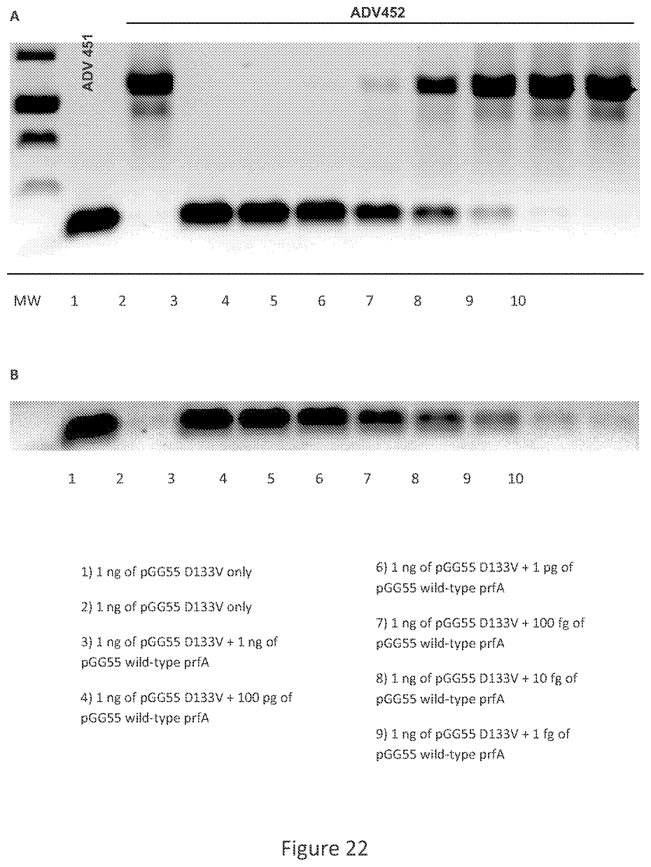

The sensitivity of the reaction was tested using 1 ng of template DNA. For the plasmid carrying the wild-type prfA sequence, decreasing amounts of DNA (corresponding to 10-fold dilutions from 10⁻¹ to 10⁻⁷), were also included in the reaction to estimate the sensitivity. In these reactions only the primers ADV452 and ADV453 were used. In a PCR reaction with 30 cycles (10 cycles with annealing temperature of 53° C. and an additional 20 cycles with annealing temperature of 50° C.), the sensitivity of the method was 1 in 100,000 (data not shown). As shown in FIG. 5, increasing the number of PCR cycles to 37 improved the visual sensitivity of the method to 10⁻⁶ for the detection of D133V revertants, without significantly compromising the specificity. A clear band was visible at the 10⁻⁶ dilution, corresponding to a detection level of 1 copy of the wild-type sequence in a million of the D133V mutant, when 1 ng of plasmid was used as the initial amount of DNA. Only a very weak band can be visualized in lanes 1 and 9 after longer exposure, reassuring the robust specificity of the method. On the other hand, when starting with 5 ng of DNA, a band could be easily detected at the 10⁻⁷ dilution, increasing the sensitivity of the PCR. However, a similar band in intensity could also be detected with the pGG55 D133V plasmid, indicating the specificity limit of the method (FIG. 17). This band observed with the pGG55 D133V plasmid is likely due to non-specific amplification of the D133V mutation with primer ADV452 that can significantly accumulate with the increased number of cycles. These results indicate that the sensitivity limit for this method, without significantly compromising the specificity, is situated between 1 to 1,000,000 and 1 to 10,000,000.

### Example 10: Recombinant Listeria Expressing a Fusion Protein of LLO to E7(Lm-LLO-E7)

This strain is approx. 4-5 logs more attenuated than the wild-type parent strain 10403S and secretes the fusion protein tLLO-E7. This immunotherapy is based on the backbone XFL7, which is derived from 10403S by the irreversible deletion in the virulence gene transcription activator prfA. PrfA regulates the transcription of several virulence genes such as Listeriolysin O (LLO), ActA, PlcA (phospholipase A), PlcB (phospholipase B) etc that are required for in vivo intracellular growth and survival of L. monocytogenes. The plasmid pGG55 is retained by the Lm-LLO-E7 in vitro by means of selection with ‘chloramphenicol’. However for in vivo retention of the plasmid by Lm-LLO-E7, it carries a copy of mutated prfA (D133V), which has been demonstrated to be less active than wild-type PrfA in DNA binding and activating the transcription of virulence genes. We have observed that complementation with mutated PrfA resulted in approx. 40 fold reduction in the amount of secreted LLO from Lm-LLO-E7 when compared to wild-type strain 10403S. This implicates that possibly the strain Lm-LLO-E7 exhibits a reduced expression of the virulence genes that are regulated by PrfA such as actA, inlA, inlB, inlC, plcB etc. In Lm-LLO-E7, the complementation with mutated copy of prfA possibly causes a reduction in the expression of different virulence genes that are regulated by PrfA resulting in overall attenuation of approx. 4-5 logs.

### Example 11: Construction of Attenuated Listeria Strain-LmddΔactA

A recombinant Lm that secretes PSA fused to tLLO (Lm-LLO-PSA) was developed, which elicits a potent PSA-specific immune response associated with regression of tumors in a mouse model for prostate cancer, wherein the expression of tLLO-PSA is derived from a plasmid based on pGG55 (Table 7), which confers antibiotic resistance to the vector. for a strain for the PSA vaccine based on the pADV142 plasmid was also developed. This strain, has no antibiotic resistance markers, and is referred as LmddA-142 (Table 7). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 7
Plasmids and strains

| Plasmids | Features |
|---|---|
| pGG55 | pAM401/pGB354 shuttle plasmid with gram(−) and |
| | gram(+) cm resistance, LLO-E7 expression cassette and a |
| | copy of LmprfA gene |
| pTV3 | Derived from pGG55 by deleting cm genes and inserting |
| | the Lmdal gene |
| pADV119 | Derived from pTV3 by deleting the prfA gene |
| pADV134 | Derived from pADV119 by replacing the Lmdal gene by |
| | the Bacillusdal gene |
| pADV142 | Derived from pADV134 by replacing HPV16 e7 with klk3 |
| pADV172 | Derived from pADV134 by replacing HPV16 e7 with hmw- |
| | maa₂₁₆₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| Lmdd-143 | 10403S dal⁽⁻⁾ dat⁽⁻⁾ with klk3 fused to the hly gene in the |
| | chromosome |
| LmddA-143 | 10403S dal⁽⁻⁾ dat⁽⁻⁾ actA⁽⁻⁾ with klk3 fused to the hly |
| | gene in the chromosome |
| LmddA-172 | 10403S dal⁽⁻⁾ dat⁽⁻⁾ actA⁽⁻⁾ pADV172 |
| Lmdd- | Lmdd-143 pADV134 |
| 143/134 | |
| LmddA- | LmddA-143 pADV134 |
| 143/134 | |
| Lmdd- | Lmdd-143 pADV172 |
| 143/172 | |
| LmddA- | LmddA-143 pADV172 |
| 143/172 | |

The sequence of the plasmid pAdv142 (6523 bp) was as follows:

(SEQ ID NO: 34)
cggagtgtatactggcttactatgttggcactgatgagggtgtcagtgaa
gtgcttcatgtggcaggagaaaaaaggctgcaccggtgcgtcagcagaat
atgtgatacaggatatattccgcttcctcgctcactgactcgctacgctc
ggtcgttcgactgcggcgagcggaaatggcttacgaacggggcggagatt
tcctggaagatgccaggaagatacttaacagggaagtgagagggccgcgg
caaagccgtttttccataggctccgcccccctgacaagcatcacgaaatc
tgacgctcaaatcagtggtggcgaaacccgacaggactataaagatacca
ggcgtttccccctggcggctccctcgtgcgctctcctgttcctgcctttc
ggtttaccggtgtcattccgctgttatggccgcgtttgtctcattccacg
cctgacactcagttccgggtaggcagttcgctccaagctggactgtatgc
acgaaccccccgttcagtccgaccgctgcgccttatccggtaactatcgt
cttgagtccaacccggaaagacatgcaaaagcaccactggcagcagccac
tggtaattgatttagaggagttagtcttgaagtcatgcgccggttaaggc
taaactgaaaggacaagttttggtgactgcgctcctccaagccagttacc
tcggttcaaagagttggtagctcagagaaccttcgaaaaaccgccctgca
aggcggttttttcgttttcagagcaagagattacgcgcagaccaaaacga
tctcaagaagatcatcttattaatcagataaaatatttctagccctcctt
tgattagtatattcctatcttaaagttacttttatgtggaggcattaaca
tttgttaatgacgtcaaaaggatagcaagactagaataaagctataaagc
aagcatataatattgcgtttcatctttagaagcgaatttcgccaatatta
taattatcaaaagagaggggtggcaaacggtatttggcattattaggtta
aaaaatgtagaaggagagtgaaacccatgaaaaaaataatgctagttttt
attacacttatattagttagtctaccaattgcgcaacaaactgaagcaaa
ggatgcatctgcattcaataaagaaaattcaatttcatccatggcaccac
cagcatctccgcctgcaagtcctaagacgccaatcgaaaagaaacacgcg
gatgaaatcgataagtatatacaaggattggattacaataaaaacaatgt
attagtataccacggagatgcagtgacaaatgtgccgccaagaaaaggtt
acaaagatggaaatgaatatattgttgtggagaaaaagaagaaatccatc
aatcaaaataatgcagacattcaagttgtgaatgcaatttcgagcctaac
ctatccaggtgctctcgtaaaagcgaattcggaattagtagaaaatcaac
cagatgttctccctgtaaaacgtgattcattaacactcagcattgatttg
ccaggtatgactaatcaagacaataaaatagttgtaaaaaatgccactaa
atcaaacgttaacaacgcagtaaatacattagtggaaagatggaatgaaa
aatatgctcaagcttatccaaatgtaagtgcaaaaattgattatgatgac
gaaatggcttacagtgaatcacaattaattgcgaaatttggtacagcatt
taaagctgtaaataatagcttgaatgtaaacttcggcgcaatcagtgaag
ggaaaatgcaagaagaagtcattagttttaaacaaatttactataacgtg
aatgttaatgaacctacaagaccttccagatttttcggcaaagctgttac
taaagagcagttgcaagcgcttggagtgaatgcagaaaatcctcctgcat
atatctcaagtgtggcgtatggccgtcaagtttatttgaaattatcaact
aattcccatagtactaaagtaaaagctgcttttgatgctgccgtaagcgg
aaaatctgtctcaggtgatgtagaactaacaaatatcatcaaaaattctt
ccttcaaagccgtaatttacggaggttccgcaaaagatgaagttcaaatc
atcgacggcaacctcggagacttacgcgatattttgaaaaaaggcgctac
ttttaatcgagaaacaccaggagttcccattgcttatacaacaaacttcc
taaaagacaatgaattagctgttattaaaaacaactcagaatatattgaa
acaacttcaaaagcttatacagatggaaaaattaacatcgatcactctgg
aggatacgttgctcaattcaacatttcttgggatgaagtaaattatgatc
tcgagattgtgggaggctgggagtgcgagaagcattcccaaccctggcag
gtgcttgtggcctctcgtggcagggcagtctgcggcggtgttctggtgca
cccccagtgggtcctcacagctgcccactgcatcaggaacaaaagcgtga
tcttgctgggtcggcacagcctgtttcatcctgaagacacaggccaggta
tttcaggtcagccacagcttcccacacccgctctacgatatgagcctcct
gaagaatcgattcctcaggccaggtgatgactccagccacgacctcatgc
tgctccgcctgtcagagcctgccgagctcacggatgctgtgaaggtcatg
gacctgcccacccaggagccagcactggggaccacctgctacgcctcagg
ctggggcagcattgaaccagaggagttcttgaccccaaagaaacttcagt
gtgtggacctccatgttatttccatgacgtgtgtgcgcaagttcaccctc
agaaggtgaccaagttcatgctgtgtgctggacgctggacagggggcaaa
agcacctgctcgggtgattctgggggcccacttgtctgttatggtgtgct
tcaaggtatcacgtcatggggcagtgaaccatgtgccctgcccgaaaggc
cttccctgtacaccaaggtggtgcattaccggaagtggatcaaggacacc
atcgtggccaaccccTAAcccgggccactaactcaacgctagtagtggat
ttaatcccaaatgagccaacagaaccagaaccagaaacagaacaagtaac
attggagttagaaatggaagaagaaaaaagcaatgatttcgtgtgaataa
tgcacgaaatcattgcttatttttttaaaaagcgatatactagatataac
gaaacaacgaactgaataaagaatacaaaaaaagagccacgaccagttaa
agcctgagaaactttaactgcgagccttaattgattaccaccaatcaatt
aaagaagtcgagacccaaaatttggtaaagtatttaattactttattaat
cagatacttaaatatctgtaaacccattatatcgggtttttgaggggatt
tcaagtctttaagaagataccaggcaatcaattaagaaaaacttagttga
ttgccttttttgttgtgattcaactttgatcgtagcttctaactaattaa
ttttcgtaagaaaggagaacagctgaatgaatatcccttttgttgtagaa
actgtgcttcatgacggcttgttaaagtacaaatttaaaaatagtaaaat
tcgctcaatcactaccaagccaggtaaaagtaaaggggctatttttgcgt
atcgctcaaaaaaaagcatgattggcggacgtggcgttgttctgacttcc
gaagaagcgattcacgaaaatcaagatacatttacgcattggacaccaaa
cgtttatcgttatggtacgtatgcagacgaaaaccgttcatacactaaag
gacattctgaaaacaatttaagacaaatcaataccttctttattgatttt
gatattcacacggaaaaagaaactatttcagcaagcgatattttaacaac
agctattgatttaggttttatgcctacgttaattatcaaatctgataaag
gttatcaagcatattttgttttagaaacgccagtctatgtgacttcaaaa
tcagaatttaaatctgtcaaagcagccaaaataatctcgcaaaatatccg
agaatattttggaaagtctttgccagttgatctaacgtgcaatcattttg
ggattgctcgtataccaagaacggacaatgtagaattttttgatcccaat
taccgttattctttcaaagaatggcaagattggtctttcaaacaaacaga
taataagggctttactcgttcaagtctaacggttttaagcggtacagaag
gcaaaaaacaagtagatgaaccctggtttaatctcttattgcacgaaacg
aaattttcaggagaaaagggtttagtagggcgcaatagcgttatgtttac
cctctctttagcctactttagttcaggctattcaatcgaaacgtgcgaat
ataatatgtttgagtttaataatcgattagatcaacccttagaagaaaaa
gaagtaatcaaaattgttagaagtgcctattcagaaaactatcaaggggc
taatagggaatacattaccattctttgcaaagcttgggtatcaagtgatt
taaccagtaaagatttatttgtccgtcaagggtggtttaaattcaagaaa
aaaagaagcgaacgtcaacgtgttcatttgtcagaatggaaagaagattt
aatggcttatattagcgaaaaaagcgatgtatacaagccttatttagcga
cgaccaaaaaagagattagagaagtgctaggcattcctgaacggacatta
gataaattgctgaaggtactgaaggcgaatcaggaaattttctttaagat
taaaccaggaagaaatggtggcattcaacttgctagtgttaaatcattgt
tgctatcgatcattaaattaaaaaaagaagaacgagaaagctatataaag
gcgctgacagcttcgtttaatttagaacgtacatttattcaagaaactct
aaacaaattggcagaacgccccaaaacggacccacaactcgatttgttta
gctacgatacaggctgaaaataaaacccgcactatgccattacatttata
tctatgatacgtgtttgtttttctttgctggctagcttaattgcttatat
ttacctgcaataaaggatttcttacttccattatactcccattttccaaa
aacatacggggaacacgggaacttattgtacaggccacctcatagttaat
ggtttcgagccttcctgcaatctcatccatggaaatatattcatccccct
gccggcctattaatgtgacttttgtgcccggcggatattcctgatccagc
tccaccataaattggtccatgcaaattcggccggcaattttcaggcgttt
tcccttcacaaggatgtcggtccctttcaattttcggagccagccgtccg
catagcctacaggcaccgtcccgatccatgtgtctttttccgctgtgtac
tcggctccgtagctgacgctctcgccttttctgatcagtttgacatgtga
cagtgtcgaatgcagggtaaatgccggacgcagctgaaacggtatctcgt
ccgacatgtcagcagacgggcgaaggccatacatgccgatgccgaatctg
actgcattaaaaaagccttttttcagccggagtccagcggcgctgttcgc
gcagtggaccattagattctttaacggcagcggagcaatcagctctttaa
agcgctcaaactgcattaagaaatagcctctttctttttcatccgctgtc
gcaaaatgggtaaatacccctttgcactttaaacgagggttgcggtcaag
aattgccatcacgttctgaacttcttcctctgtttttacaccaagtctgt
tcatccccgtatcgaccttcagatgaaaatgaagagaaccttttttcgtg
tggcgggctgcctcctgaagccattcaacagaataacctgttaaggtcac
gtcatactcagcagcgattgccacatactccgggggaaccgcgccaagca
ccaatataggcgccttcaatccctttttgcgcagtgaaatcgcttcatcc
aaaatggccacggccaagcatgaagcacctgcgtcaagagcagcctttgc
tgtttctgcatcaccatgcccgtaggcgtttgctttcacaactgccatca
agtggacatgttcaccgatatgttttttcatattgctgacattttccttt
atcgcggacaagtcaatttccgcccacgtatctctgtaaaaaggttttgt
gctcatggaaaactcctctctttttcagaaaatcccagtacgtaattaag
tatttgagaattaattttatattgattaatactaagtttacccagttttc
acctaaaaaacaaatgatgagataatagctccaaaggctaaagaggacta
taccaactatttgttaattaa.

This plasmid was sequenced at Genewiz facility from the E. coli strain on 2-20-08.

### Example 12: Insertion of the Human klk3 Gene in Frame to the hly Gene in the Lmdd and Lmndda Strains

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal datAactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 8. The upstream and downstream DNA regions of actA were cloned in the pNEB193 at the EcoRI/PstIrestriction site and from this plasmid, the EcoRI/PstIwas further cloned in the temperature sensitive plasmid pKSV7, resulting in ΔactA/pKSV7 (pAdv120).

TABLE 8
Sequence of primers that was used for the
amplification of DNA sequences upstream and
downstream of actA

| | | SEQ |
|---|---|---|
| | | ID |
| Primer | Sequence | NO: |
| Adv271-actAF1 | cgGAATTCGGATCCgcgccaaatcattggtt | 35 |
| | gattg | |
| Adv272-actAR1 | gcgaGTCGACgtcggggttaatcgtaatgca | 36 |
| | attggc | |
| Adv273-actAF2 | gcgaGTCGACccatacgacgttaattcttgc | 37 |
| | aatg | |
| Adv274-actAR2 | gataCTGCAGGGATCCttcccttctcggtaa | 38 |
| | tcagtcac | |

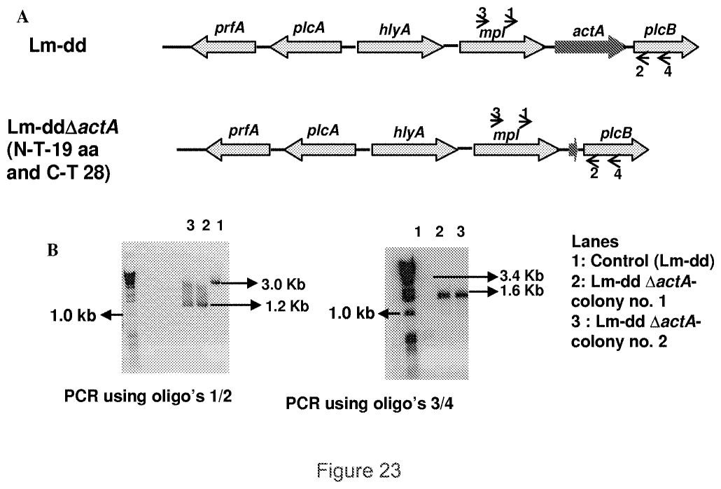

The deletion of the gene from its chromosomal location was verified using primers that bind externally to the actA deletion region, which are shown in FIG. 23 as primer 3 (Adv 305-tgggatggccaagaaattc, SEQ ID NO: 39) and primer 4 (Adv304-ctaccatgtcttccgttgcttg; SEQ ID NO: 40). The PCR analysis was performed on the chromosomal DNA isolated from Lmdd and LmddΔactA. The sizes of the DNA fragments after amplification with two different sets of primer pairs 1/2 and 3/4 in Lmdd chromosomal DNA was expected to be 3.0 Kb and 3.4 Kb. On the other hand, the expected sizes of PCR using the primer pairs 1/2 and 3/4 for the LmddΔactA was 1.2 Kb and 1.6 Kb. Thus, PCR analysis in FIG. 23 confirms that the 1.8 kb region of actA was deleted in the LmddΔactA strain. DNA sequencing was also performed on PCR products to confirm the deletion of actA containing region in the strain, LmddΔactA.

### Example 13: Construction of the Antibiotic-Independent Episomal Expression System for Antigen Delivery by Lm Vectors

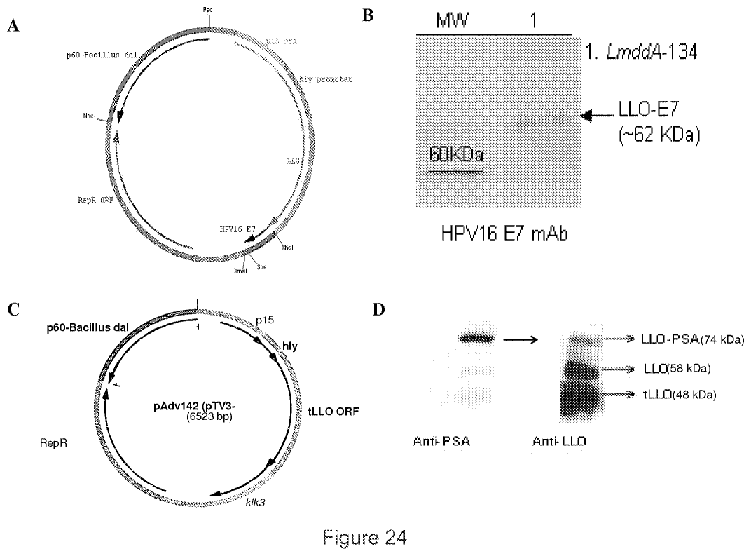

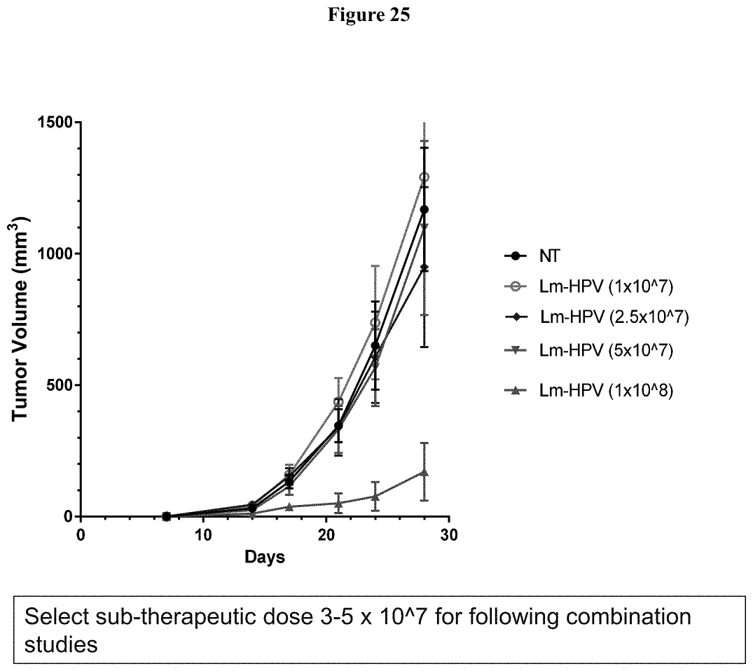

The antibiotic-independent episomal expression system for antigen delivery by Lm vectors (pAdv142) is the next generation of the antibiotic-free plasmid pTV3 (Verch et al., Infect Immun, 2004. 72 (11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 24A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 24B). The Lmdd system derived from the 10403S wild-type strain lacks antibiotic resistance markers, except for the Lmdd streptomycin resistance.

Further, pAdv134 was restricted with XhoI/XmaI to clone human PSA, k/k3 resulting in the plasmid, pAdv142. The new plasmid, pAdv142 (FIG. 24C, Table 7) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 24C).

The plasmid pAdv142 was transformed to the Listeria background strains, Lmdd actA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 24D). There was stable expression and secretion of LLO-PSA fusion protein by the strain, Lm-ddA-LLO-PSA after two in vivo passages.

### Example 14: Combination of Listeria-Based HPV-E7 Cancer Vaccine with Anti-Cd137 Agonistic Antibody or Anti-CTLA-4 Blocking Antibody Provides an Effective Immunotherapy to HPV+ Tumor Regression and Survival in a Mouse Model

Axalimogene filolisbac (AXAL), also referred to as Lm-HPV, Lm-LLO-E7, ADXS11-001, is a live attenuated Listeria monocytogenes (Lm)-based immunotherapy that expresses the full length E7 protein of human papillomavirus (HPV) 16. AXAL currently being investigated in multiple clinical trials for cervical (Phase-3), anal (Phase-2) and head & neck cancer (Phase-1/2) either as a mono therapy or in combination with checkpoint inhibitors (PD-1 or PD-L1) (Mkrtichyan M, et al., “Anti-PD-1 antibody significantly increases therapeutic efficacy of Listeria monocytogenes (Lm)-LLO immunotherapy”. J ImmunoTherapy of Cancer 2013, 1:15). This Listeria-based immunotherapy acts by inducing the de novo generation of tumor antigen-specific T cells that infiltrate and destroy the tumor and by reducing the numbers and activities of immunosuppressive regulatory T cells (Tregs) and myeloid-derived suppressor cells (MDSCs) in the tumor microenvironment. In order to identify additional immune modulators which may have synergy with AXAL, in this study we evaluated antibodies (Abs) for T cell co-inhibitory or co-stimulatory receptors (checkpoint inhibitors CTLA-4, PD-1, TIM-3, LAG3 and co-stimulators CD137, OX40, GITR and CD40) with or without AXAL in a HPV+TC1 syngeneic mouse tumor model. Results of tumor growth inhibition (TGI) and survival were compared and the antibodies+AXAL with superior performance were further characterized by immune phenotyping the tumor microenvironment (TME).

### Materials and Methods

C57BL/6 female mice (6-8 wk old) were purchased from Jackson Laboratories. TC1 cells, derived from C57BL/6 mouse lung epithelial cells and co-transfected with HPV16 E6 & E7 and activated ras oncogene, were obtained from ATCC. To establish primary tumors, 2×10 TC-1 cells were injected subcutaneously in the mice hind flank and allowed to grow for 8 to 11 days prior to the start of treatment.

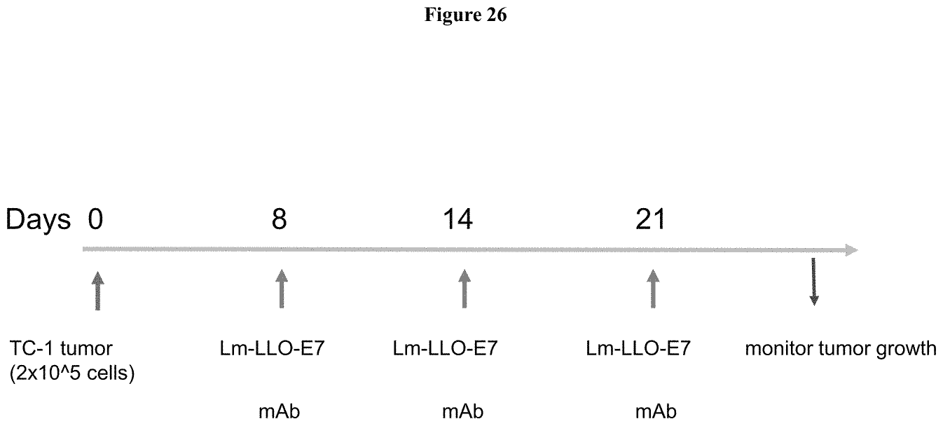

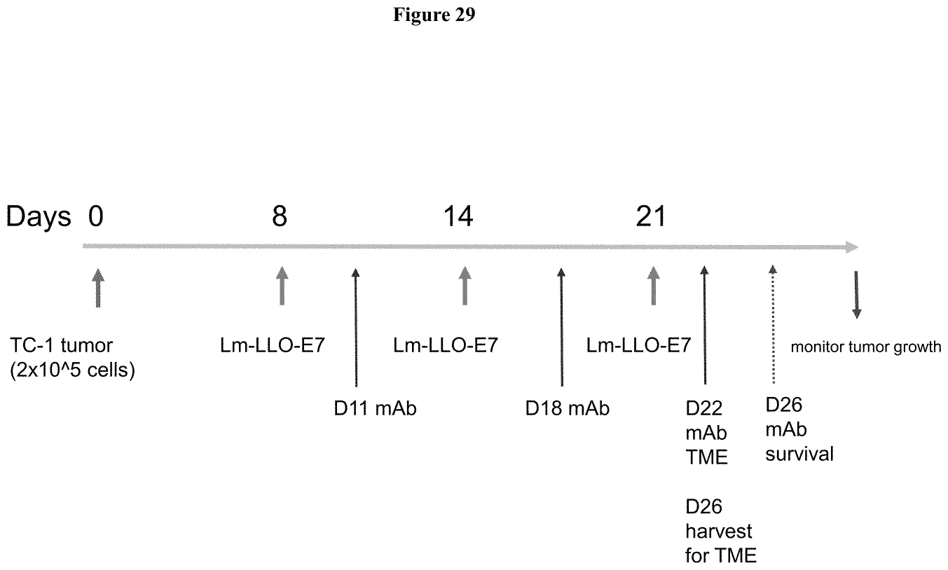

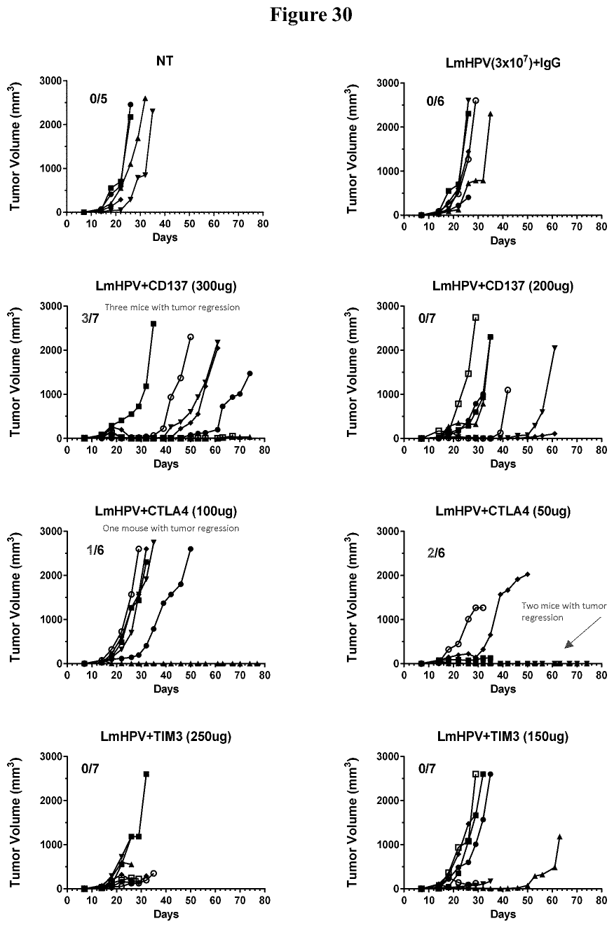

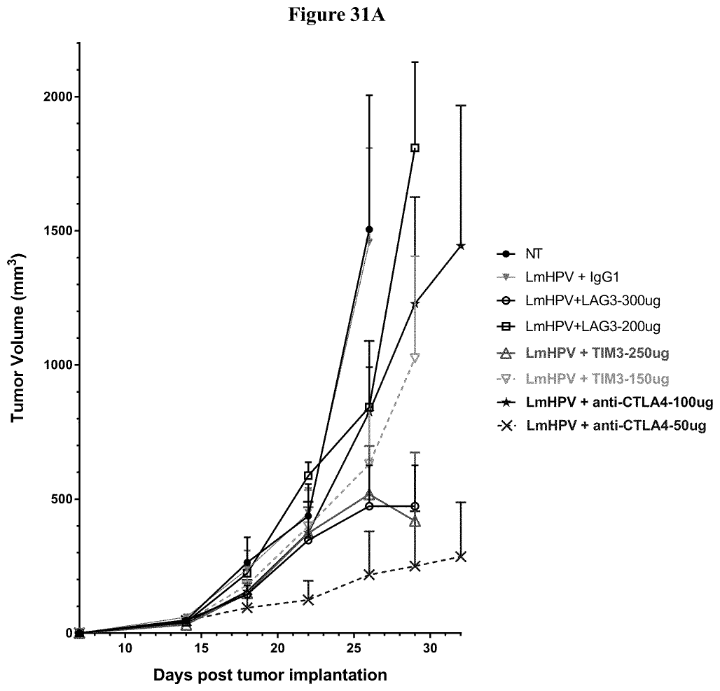

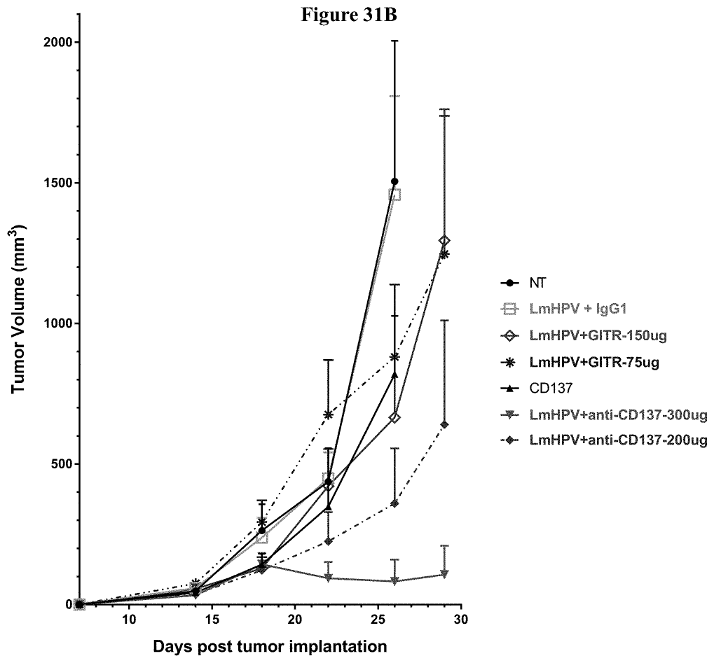

To reveal potential synergy between AXAL and antibody-based immunotherapies, a dose titration was performed to determine a subtherapeutic dose of AXAL. A dose range of 3 to 5×10⁷ CFU was found to exhibit a nominal therapeutic benefit. Lm-tLLO, an Lm vector that does not express any tumor-associated protein, was used as a vector control. Lm-HPV vaccine was injected intraperitoneally (i.p.) at 3-5×107 CFU/mouse weekly for total 3 doses. Abs were either administered simultaneously with the Lm-HPV vaccine (FIG. 26) or staggered from the administration of the Lm-HPV vaccine (FIG. 29). All Abs were obtained from BioXcell (Lebanon, N.H.) and given to mice i.p. The clone names and the concentration(s) tested for each antibody are listed in Table 9. Flow cytometry was used to immune phenotype the tumor infiltrating lymphocytes (TILs), spleen and tumor-draining lymph node (TDLN).

TABLE 9
Clone names and concentrations of antibodies used in study

| Antibody Target | Clone | Concentration(s) Tested (μg/dose) |
|---|---|---|
| CD40 | FGK4.5 | 100 |
| CD137 | LOB12.3 | 200/300 |
| CTLA-4 | 9H10 | 50/100 |
| LAG3 | C9B7W | 200/300 |
| TIM3 | RMT3-23 | 150/250 |

### Results

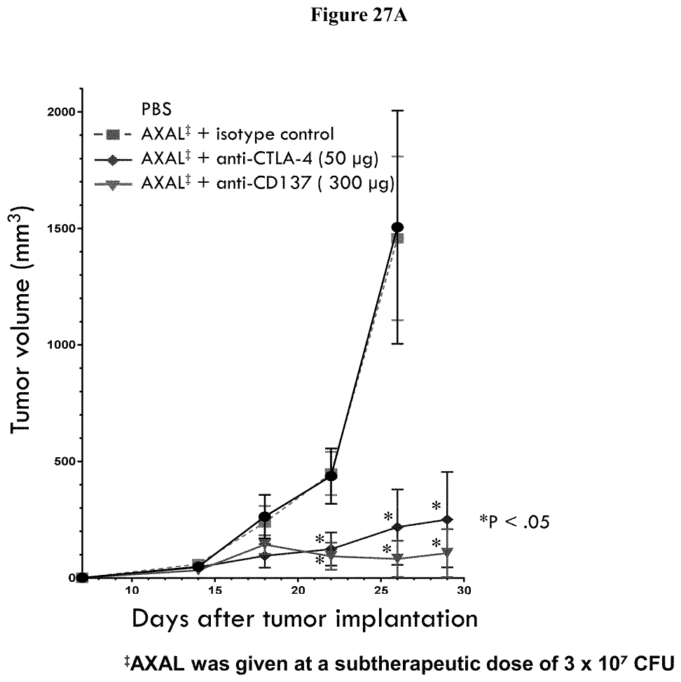

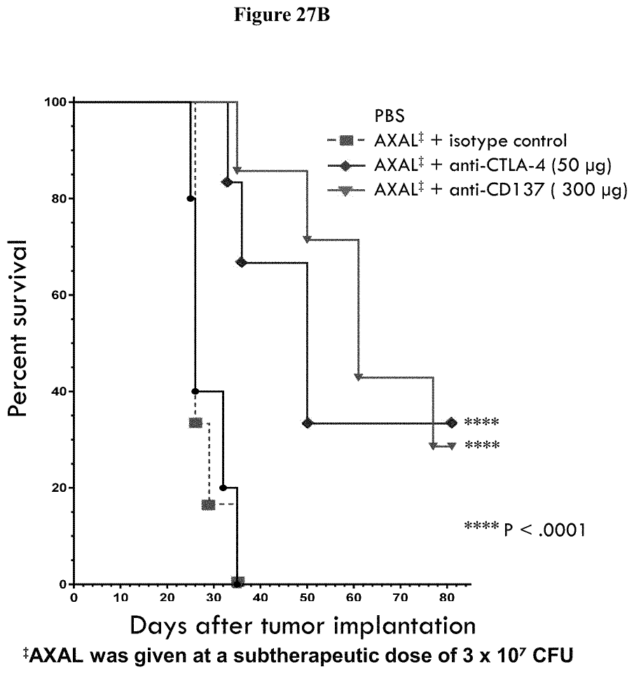

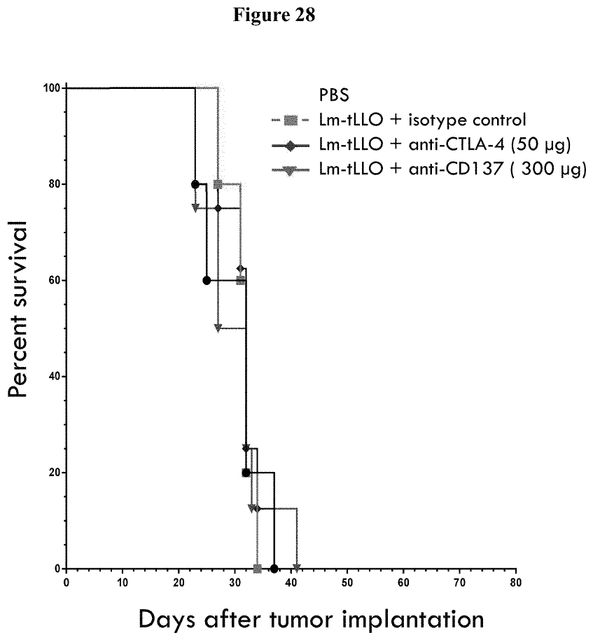

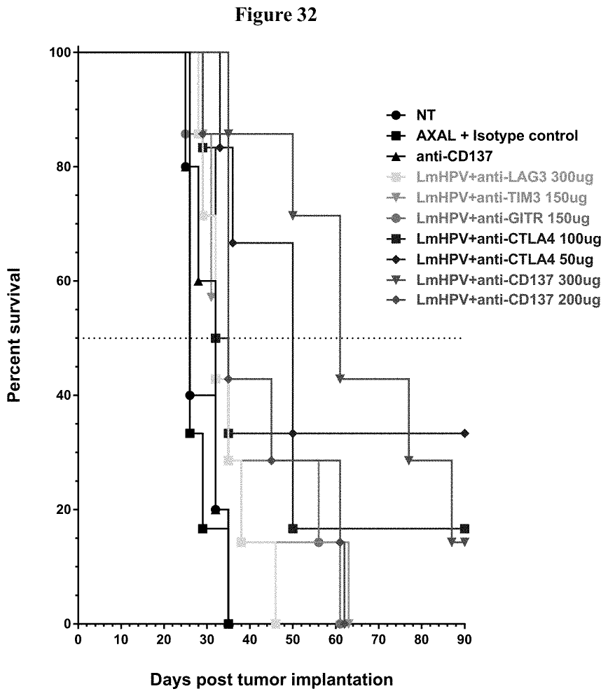

Of the 5 mAbs tested, anti-CD137 mAb and anti-CTLA-4 mAb were the most effective at synergizing with AXAL to eradicate established TC-1 tumors and to provide long-term survival (ie, >80 days post tumor implantation). Complete tumor regression was observed in 28% of the mice treated with AXAL+anti-CD137 mAb and in 33% of the mice treated with AXAL+anti-CTLA-4 mAb (FIGS. 27A and 27B). These anti-tumor effects were dependent upon the expression of the HPV-E7 protein, as mice receiving anti-CD137 mAb or anti-CTLA-4 mAb in combination with the Lm-tLLO vector (which does not express any tumor-associated protein) had survival curves similar to the survival curve of PBS-treated mice (FIG. 28). Staggered administration (FIG. 29) wherein the first dose of mAb is administered about 72 hours after the administration of the first dose of AXAL produced the highest percent survival compared to other dosing schemes (FIG. 32). The subtherapeutic doses of AXAL, anti-CD137 mAb, and anti-CTLA-4 mAb, all have no therapeutic benefit when given alone. Surprisingly, the data shows that when a combination of AXAL and either anti-CD137 or anti-CTLA-4 mAb is given, there is a synergistic effect which provides effective anti-tumor immunity in a mouse TC-1 tumor model. This synergistic effect is particularly prevalent in the staggered administration dosing scheme. Without being bound to a particular theory, the administration of AXAL before the administration of the mAb may serve as an activating agent. CD137 and CTLA-4 expression, for example, may be induced upon T cell activation. Therefore, administering the anti-CD137 mAb or anti-CTLA-4 mAb about 72 hours after the administration of AXAL may allow for the generation of more cells expressing the target receptor (i.e., CD137 and CTLA-4) and thereby amplifying the effect of the mAb.

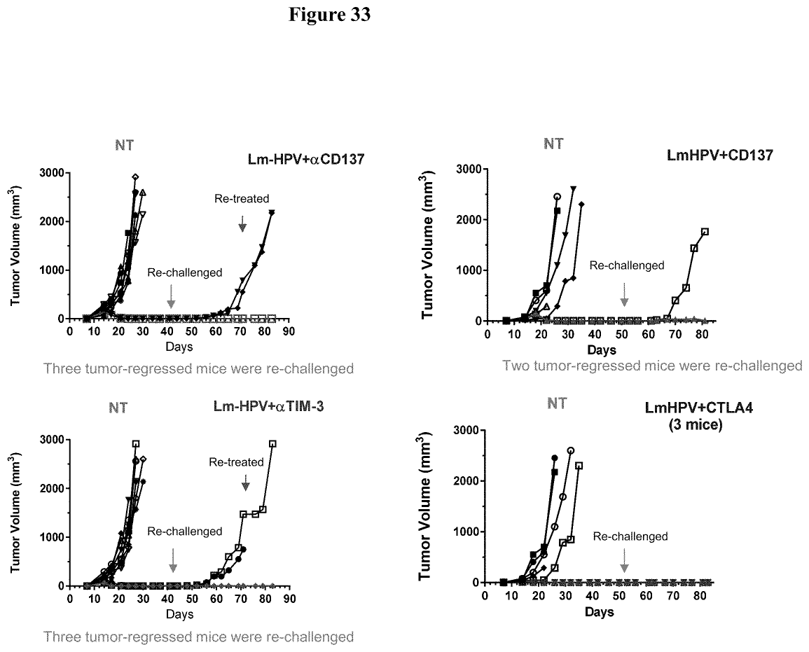

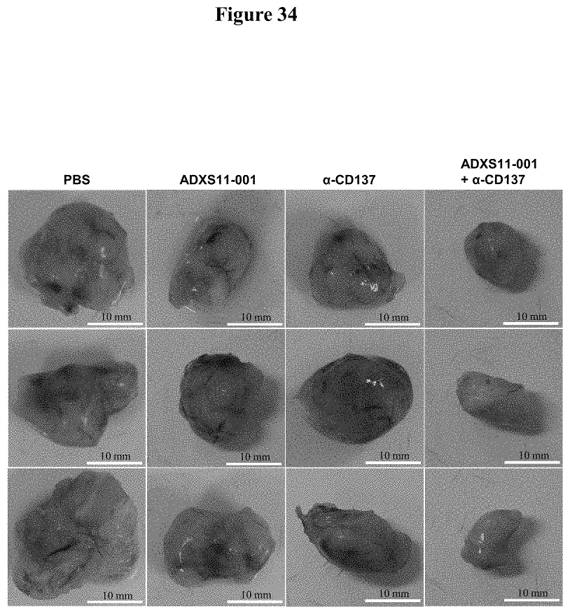

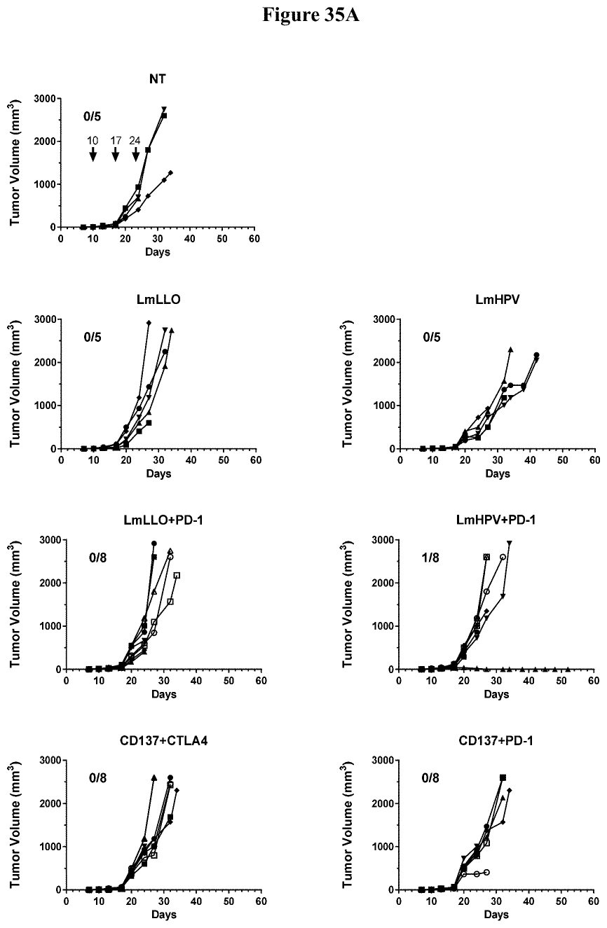

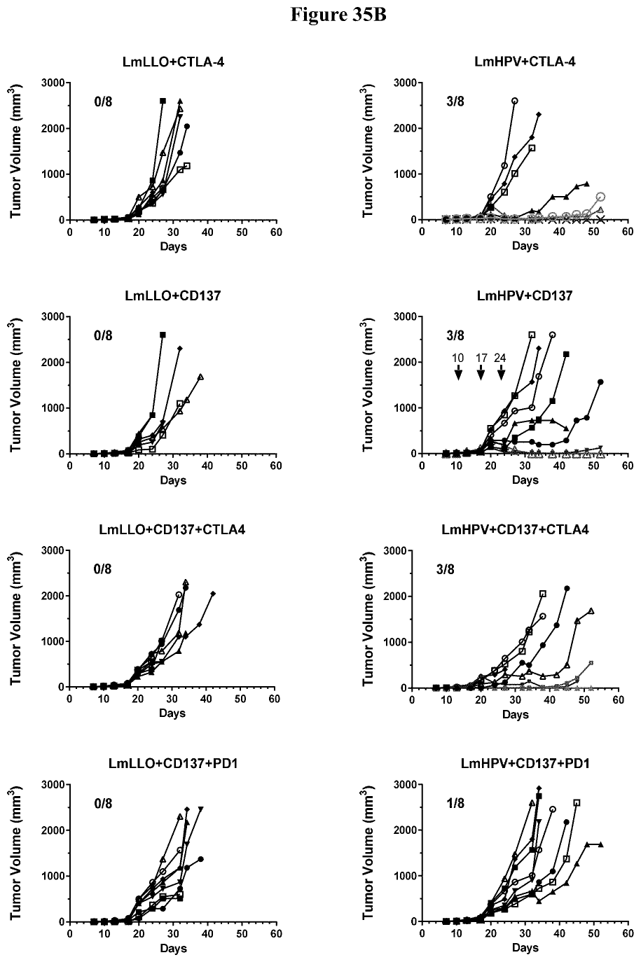

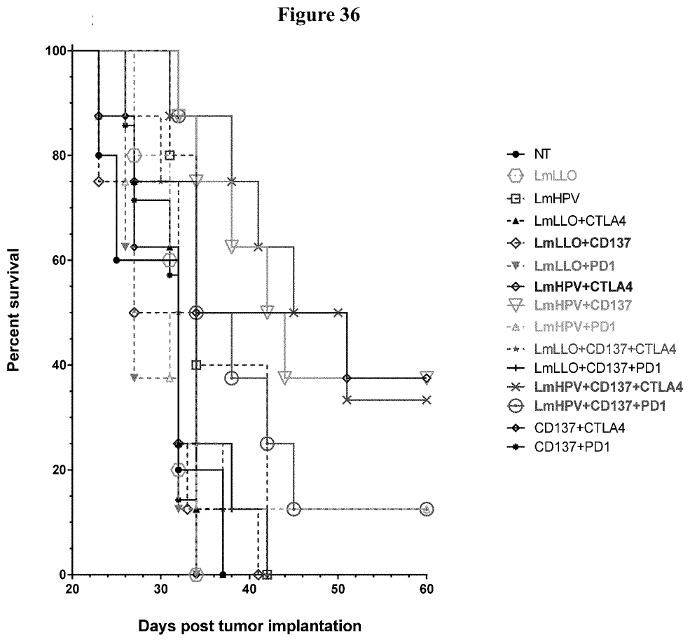

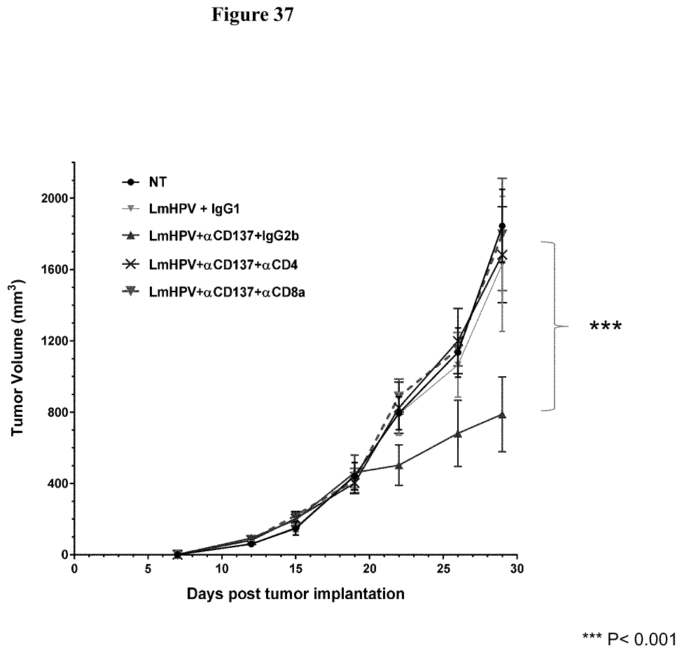

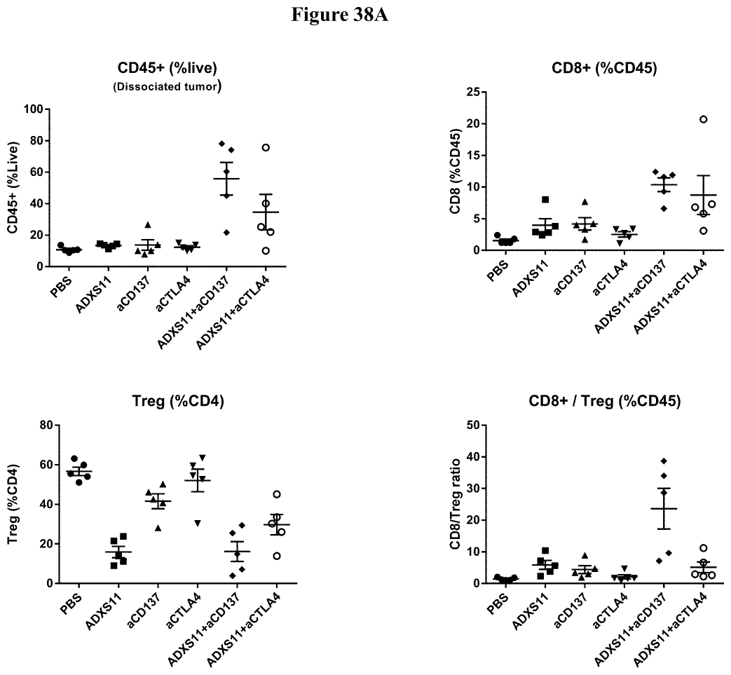

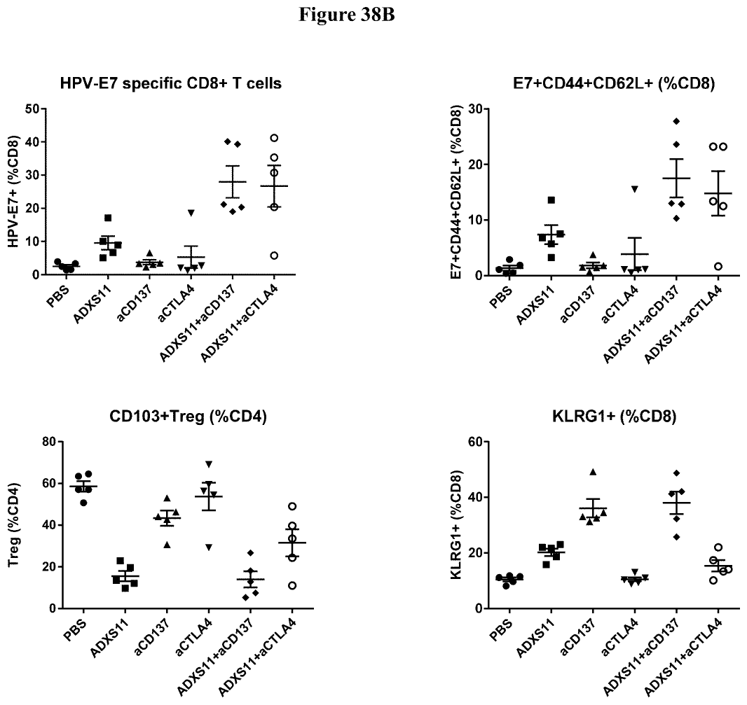

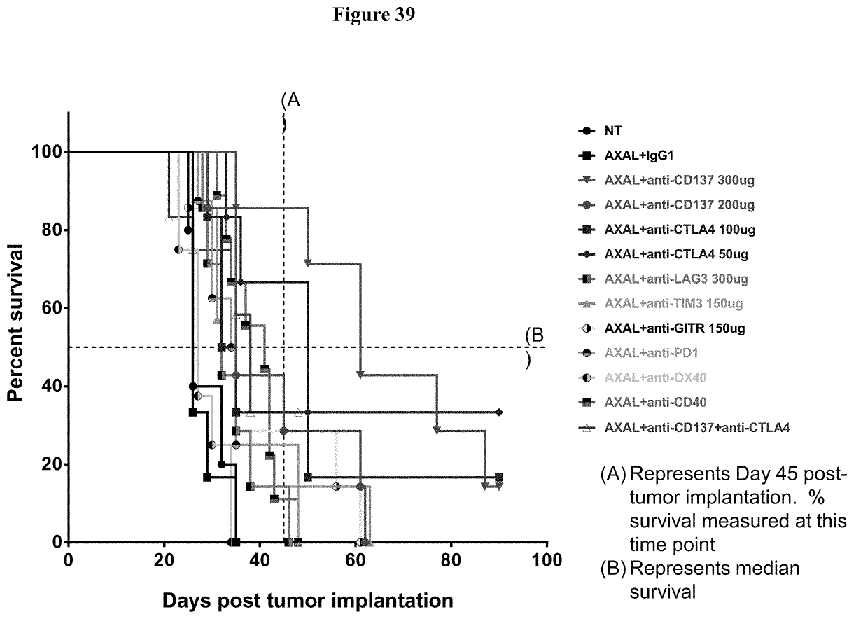

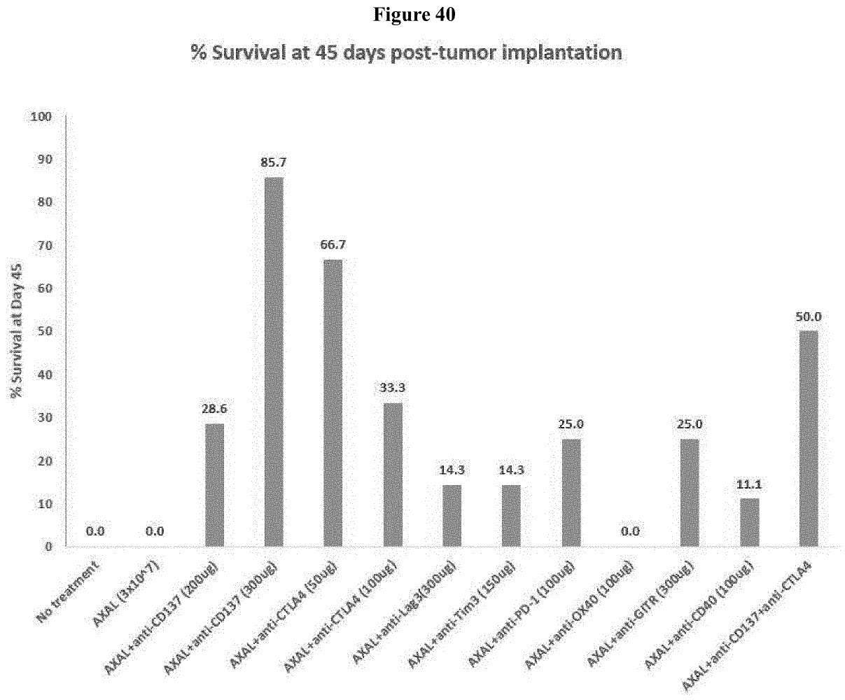

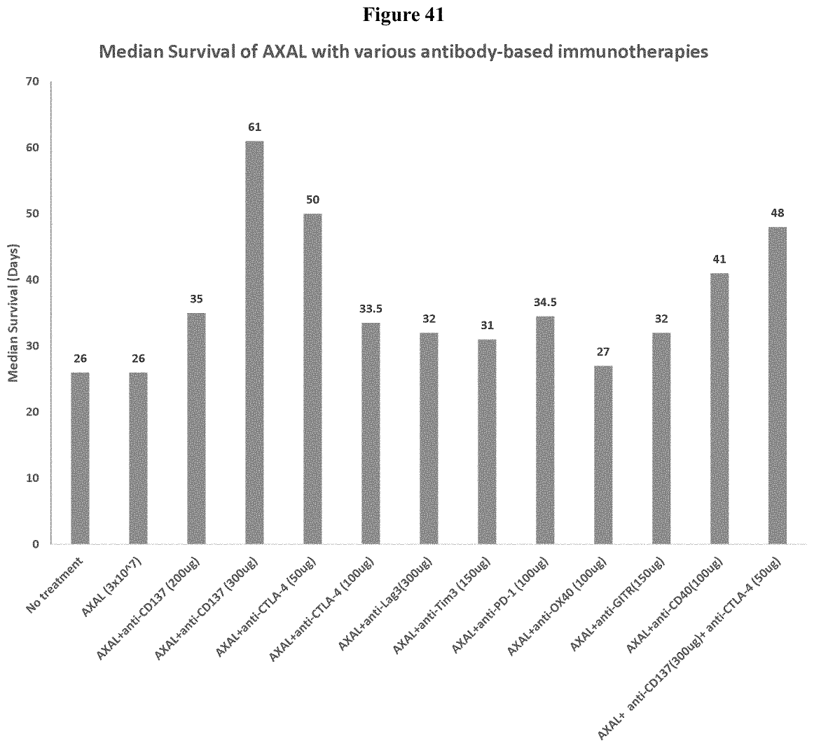

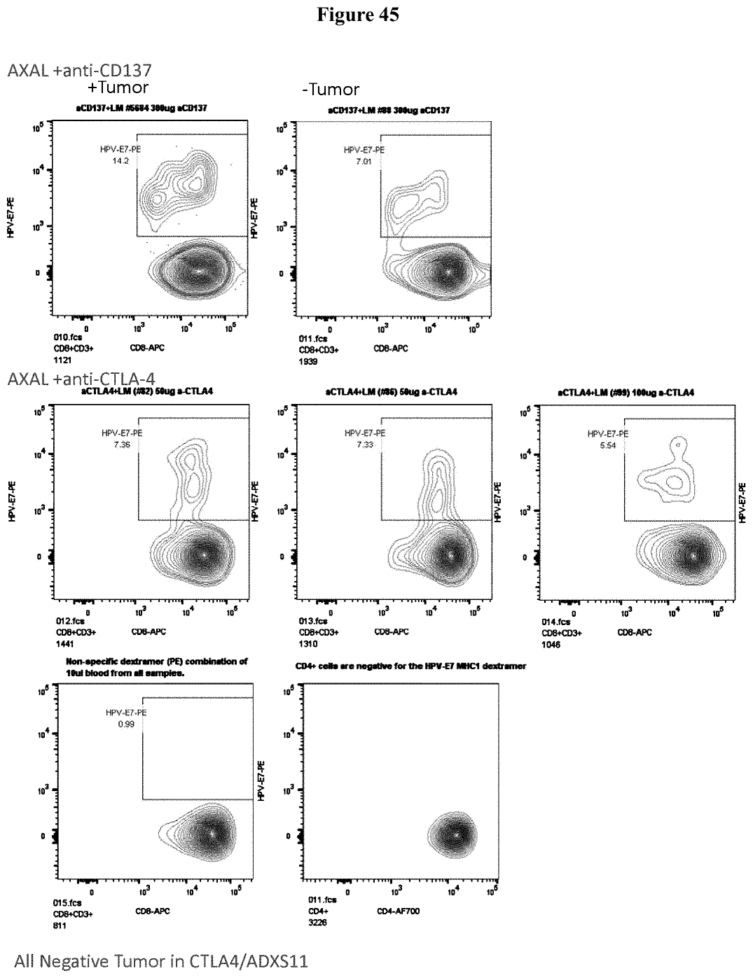

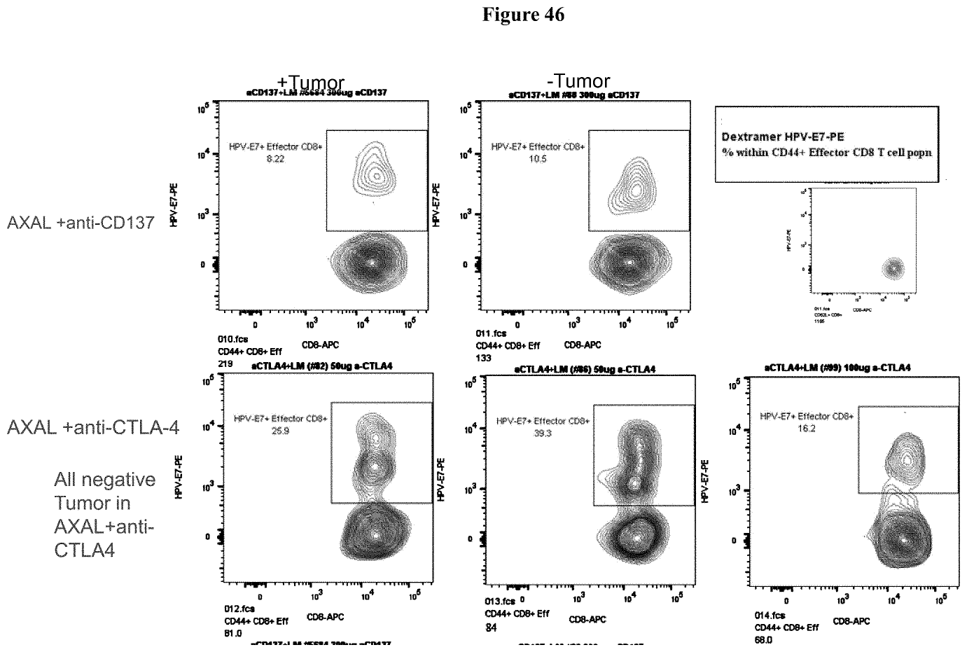

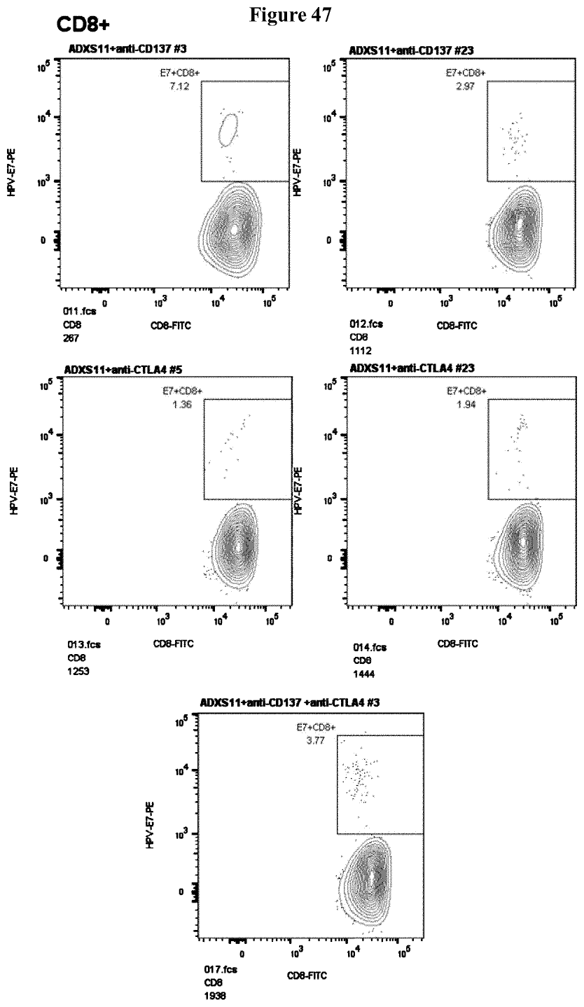

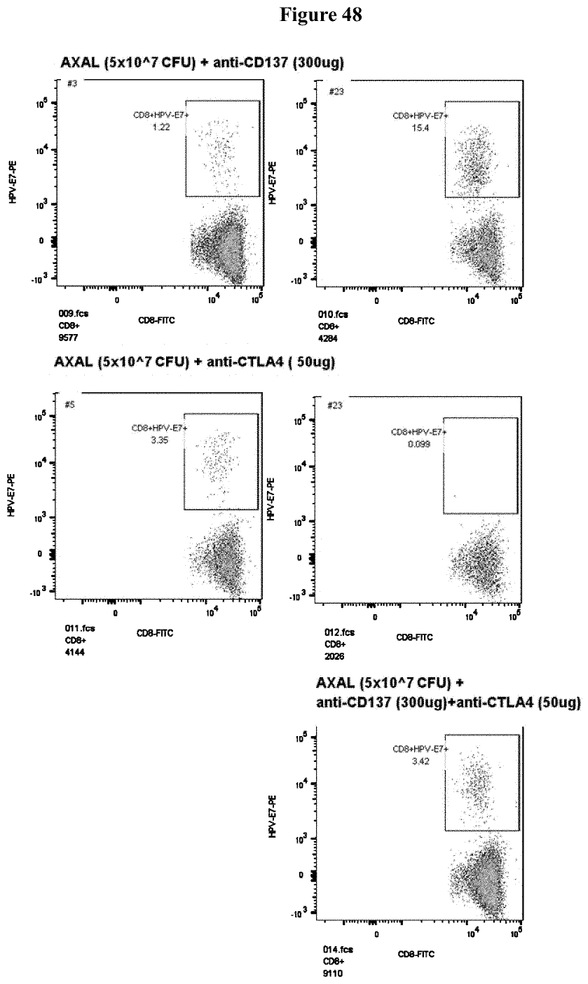

Combination therapy with CD137 antibody generates a larger percentage of CD8+ T cells than other treatments (FIG. 39A) and would be expected to generate a larger percentage of memory T cells as well. Mice with complete tumor regression after CD137+Lm-HPV treatment, were re-challenged 6-7 weeks post-tumor implantation with TC-1 cells. Of the five mice re-challenged, two mice remained tumor-free for an additional 6-7 weeks until the study was terminated; other three had delayed or slower tumor growth compared with the untreated control group. CTLA-4+Lm-HPV treatment resulted in 3 mice with complete tumor regression. These mice were re-challenged 7 weeks post-tumor implantation and remain tumor-free (FIG. 33). This suggests that combination treatment with CTLA-4 generates a larger population of memory T cells compared to other treatments. The presence of HPV-E7 specific CD8+ T Cells in blood of re-challenged mice was analyzed and results are shown in FIGS. 45-48 and Table 10 below.

TABLE 10
Percentage of HPV-E7-specificCD8 T cells 3 weeks after rechallenge

| | HPV-E7+ | |
|---|---|---|
| | CD8 Effector (% | HPV-E7+ |
| SampleID | CD8) | CD8+ (% CD8) |
| AXAL + anti-CD137 #3 | 1.08 | 1.22 |
| AXAL + anti-CD137 #23 | 14.8 | 15.4 |
| AXAL + anti-CTLA4 #5 | 2.90 | 3.35 |
| AXAL + anti-CTLA4 #23 | 0.049 | 0.099 |
| AXAL + anti-CD137 + anti- | 3.00 | 3.42 |
| CTLA4 | | |

AXAL + anti-CTLA-4 #23 tumor regrew after rechallenge. Observed very few E7+ CD8+ T cells.

Furthermore, immune phenotyping the TME in the CD137+Lm-HPV treatment group showed increased TILs, CD8/Treg ratio, and decreased highly immune-suppressive CD103+Tregs compared to treatment with either single agent alone. Additionally, increased PD-L1 expression on tumor cells and increased PD-1 expression on CD8+ T-cells was observed. The following are upregulated by LmHPV & LmHPV+CD137: complement. chemokine. integrin and adhesion, inner immunity (i.e., TLRs, NK, NKG2D, Granzyme). IFN regulation and signaling, interleukins & signaling (IL-1Ra, IL-4R, IL-10Ra, IL-12, IL17Ra, IL-18, CSF-1 & R, CSF2RB, STAT, JAKs, Lck, MAPK), T. B, DC, NK cell co-stim or checkpoint: CTL-4, ICOS, CD27, CD86, HVEM, BTLA, CD40, TGFb and its receptor, markers: F4/80, FoxP3.

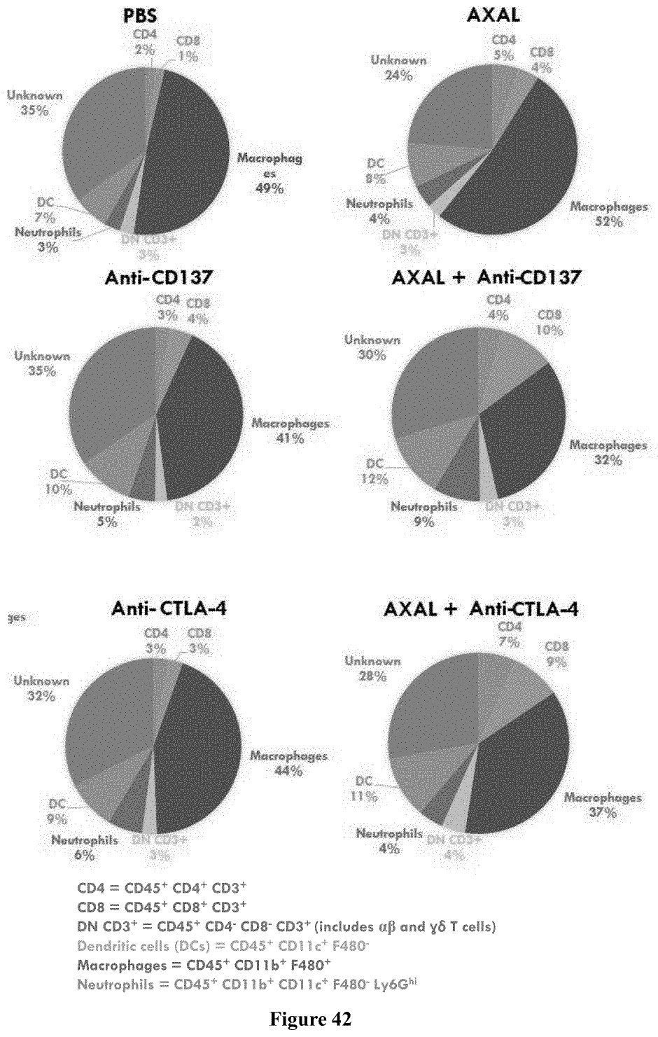

To elucidate the mechanism(s) by which AXAL+anti-CD137 mAb and AXAL+anti-CTLA-4 mAb mediate tumor control, phenotypic analysis of the tumor-infiltrating cells was performed on day 25, 7 days after the last AXAL and/or mAb treatment. The distribution of immune cell types within the tumor differed slightly between the two combination therapies (FIG. 42). With the combination of AXAL+anti-CD137 mAb, there were increases in the percentages of CD8⁺ T cells, dendritic cells (DCs), and neutrophils compared with AXAL alone, anti-CD137 mAb alone, or PBS treatment. With the combination of AXAL+anti-CTLA-4 mAb, there were increases in the percentages of CD8⁺ T cells, CD4⁺ T cells, and DCs compared with AXAL alone, anti-CTLA-4 mAb alone, or PBS treatment. Notably, both AXAL+anti-CD137 mAb and AXAL+anti-CTLA-4 mAb reduced the percentage of macrophages in the tumor compared with the single-agent therapies.

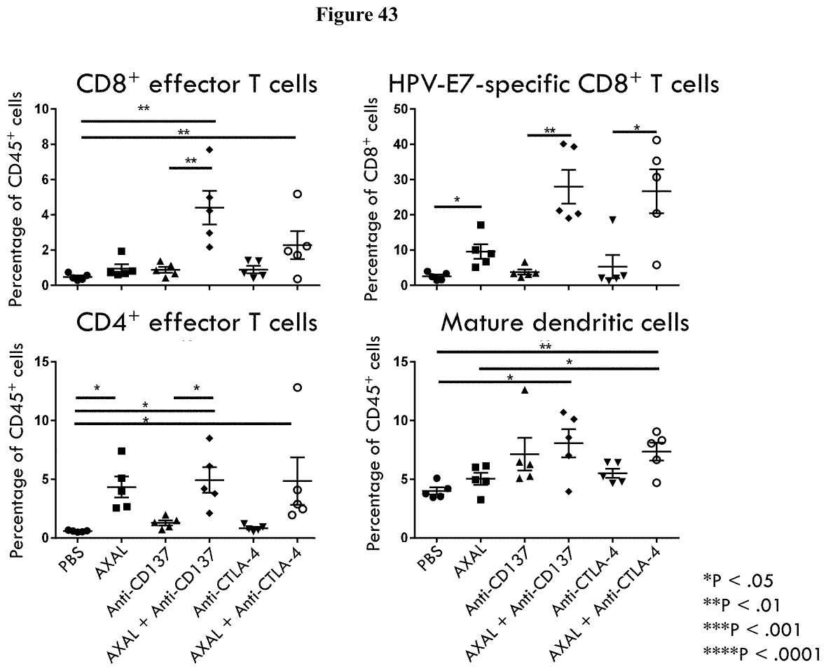

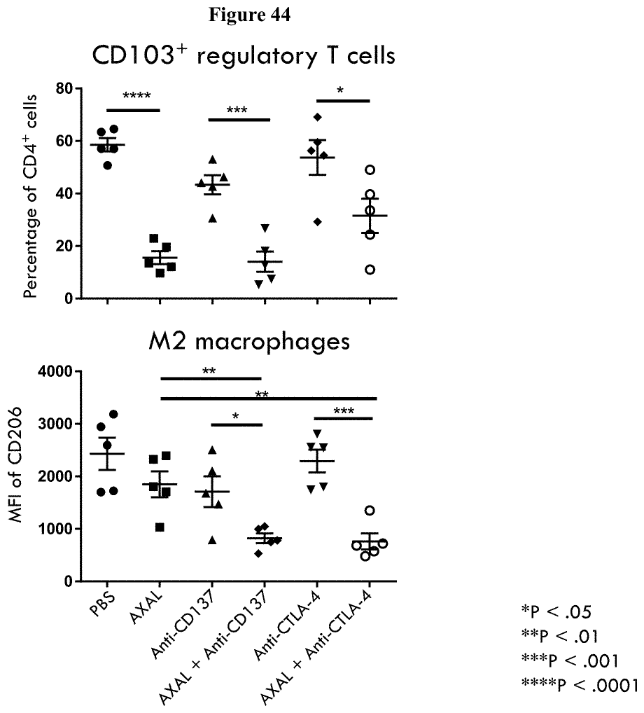

Quantitation of the relative percentages of effector and suppressor cell subsets in the tumor revealed similarities between the two combination therapies (FIGS. 43 and 44). Specifically, increases in the percentages of total effector CD8⁺ T cells and HPV-E7-specific CD8⁺ T cells, increase in the percentage of total effector CD4⁺ effector T cells, increase in the percentages of mature (ie, CD86⁺ MHC Class II⁺) DCs, decrease in the percentages of CD103⁺ regulatory T cells (CD4⁺ CD25⁺ Foxp3⁺), and decrease in the percentage of immunosuppressive M2 macrophages (based on CD206 expression levels, which are measured by mean fluorescence intensity or MFI).

The combined immunotherapy demonstrated superior antitumor efficacy with prolong survival and tumor regression in a HPV+ tumor mouse model.

### Example 15: CD137 as a Target for Combination Therapy with Advaxis' Lm-Based Immunotherapies

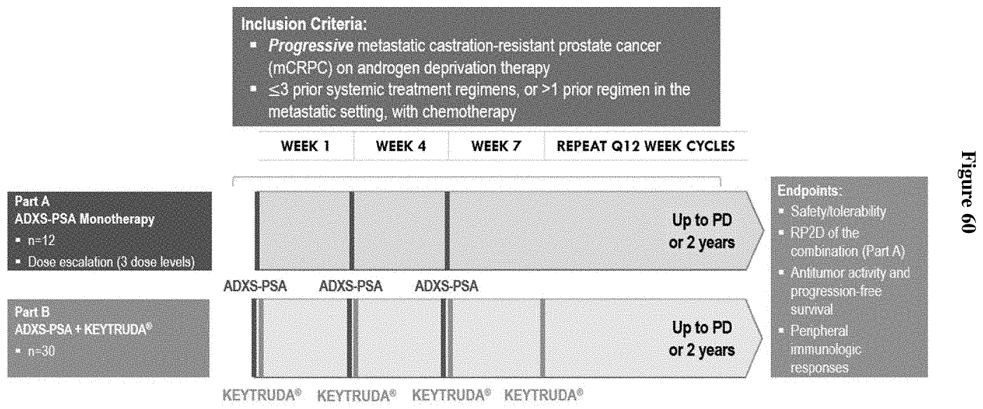

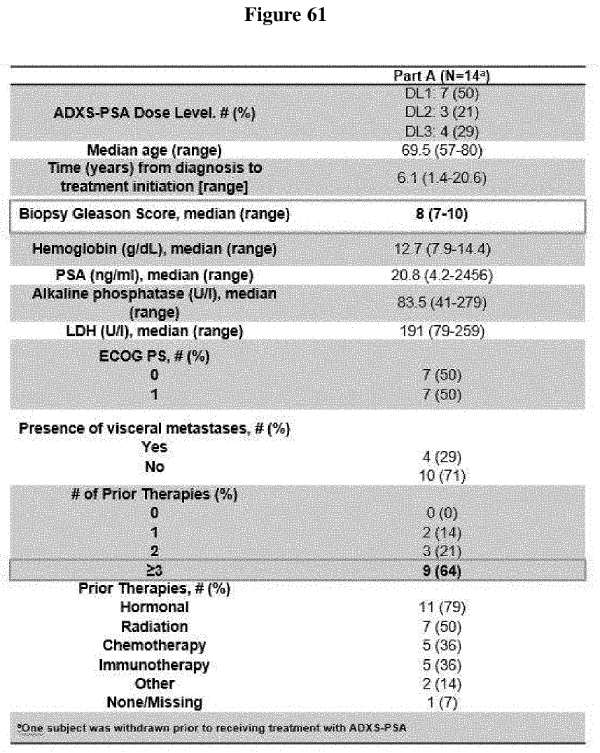

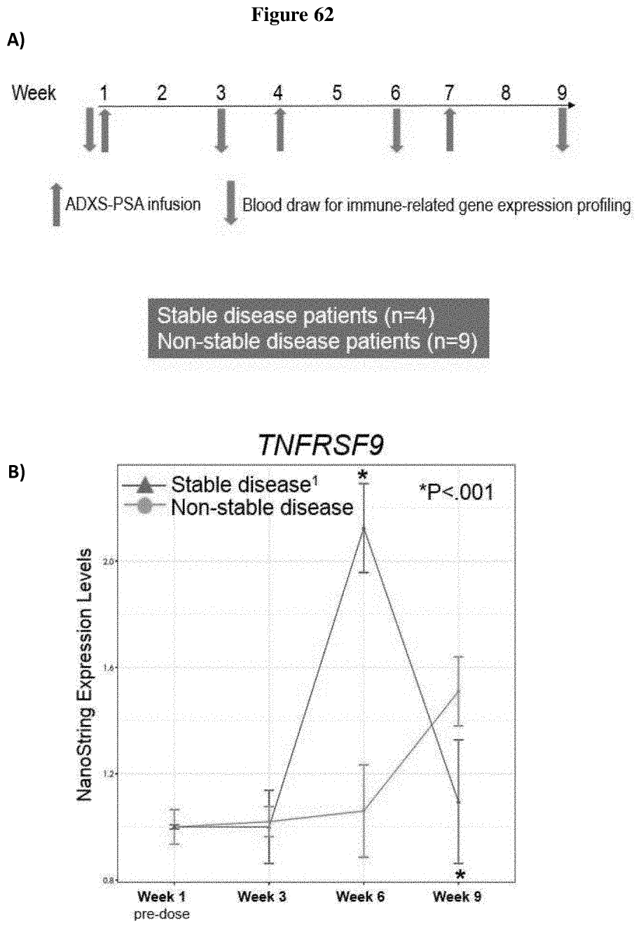

Peripheral blood mononuclear cells (PBMCs) of subjects from the ADXS-PSA monotherapy arm of the KEYNOTE-046 trial (FIG. 60) were analyzed for expression levels of TNFSF9 and PDCDJ (FIG. 62A). Key baseline demographics of study participants in Part A are shown in FIG. 61.

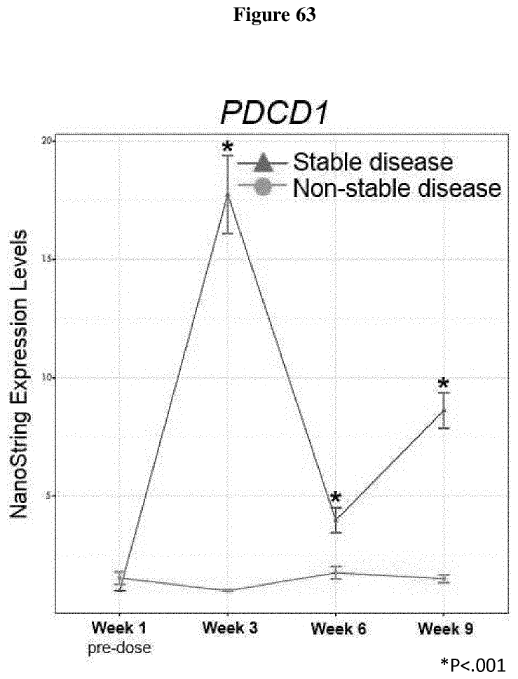

ADXS-PSA monotherapy upregulates expression of TNFRSF9, the gene encoding CD137, in all ADXS-PSA-treated mCRPC patients (i.e., stable disease and non-stable disease metastatic castration-resistant prostate cancer (mCRPC) patients) (FIG. 62B). In contrast to TNFRSF9, only stable disease patients upregulate expression of PDCD1, the gene encoding PD-1, following ADXS-PSA treatment (FIG. 63).

This finding suggests that combining Lm-based immunotherapies (e.g., ADXS-PSA) with an agonistic anti-CD137 mAb may provide clinical benefit to more cancer patients.

### Example 16: Effects of Combining AXAL with Anti-CD137 mAbs on Tumor Growth and Animal Survival

To evaluate the efficacy of Lm-based immunotherapies alone or in combination with another immunotherapy in a murine human papillomavirus (HPV)+ tumor model, the effects of combining AXAL and anti-CD137 on tumor growth and animal survival was studied. Axalimogene filolisbac (AXAL) is a live attenuated Listeria monocytogenes (Lm)-based immunotherapy that expresses and secretes the full length E7 protein of HPV 16.

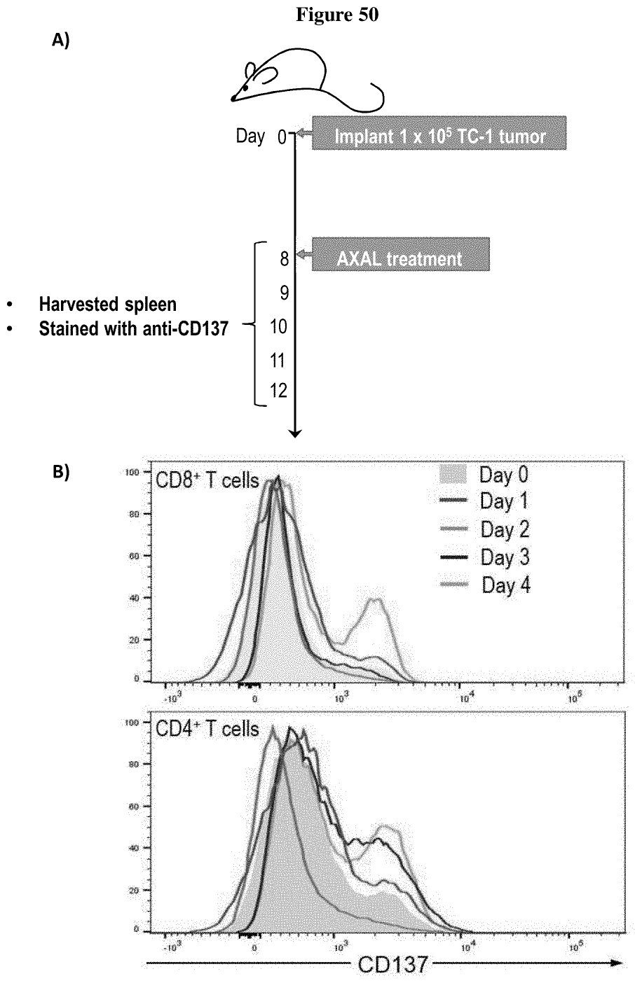

The timing of anti-CD137 mAb treatment when combined with AXAL was optimized based on the kinetics of CD137 expression on T cells following AXAL treatment (FIGS. 50A and B). It was found that AXAL treatment induces CD137 expression on both CD4 and CD8 T cells within 4 days.

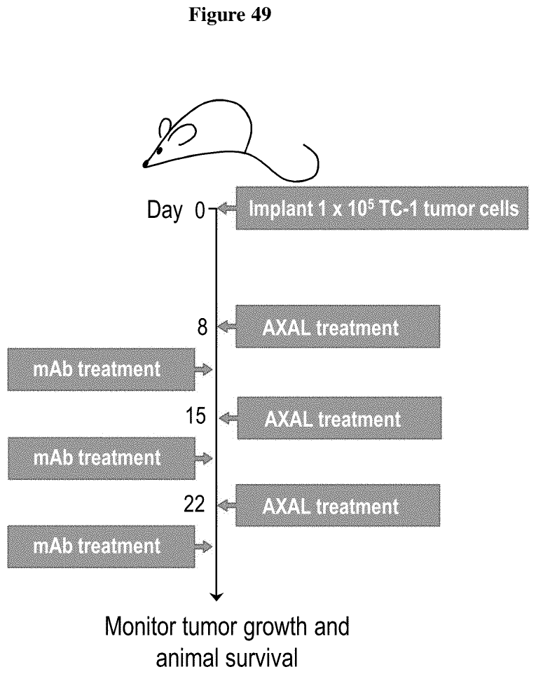

On Day 0 mice were injected with 1×10⁵ TC-1 tumor cells that were tested to be negative for mycoplasma. Starting on Day 11, the mice were administered Lm-treatment and mAb treatment according to the administration schedule in FIG. 49. TC-1 tumor cells are derived from a C57BL/6 lung epithelial cell line that was immortalized with E6 and E7 of HPV 16 and transformed with an activated ras oncogene.

Starting on Day 11, the mice were administered Lm-treatment and mAb treatment according to the administration schedule in FIG. 49.

The groups tested in the experiment were as follows:

PBS

XLF7 (1E8)+isotype

AXAL (1E8)+isotype

Anti-CD137 (150 μg)*

Anti-CD137 (300 μg)

AXAL+anti-CD137 (150 μg)

AXAL+anti-CD137 (300 μg)

*Clone LOB 12.3

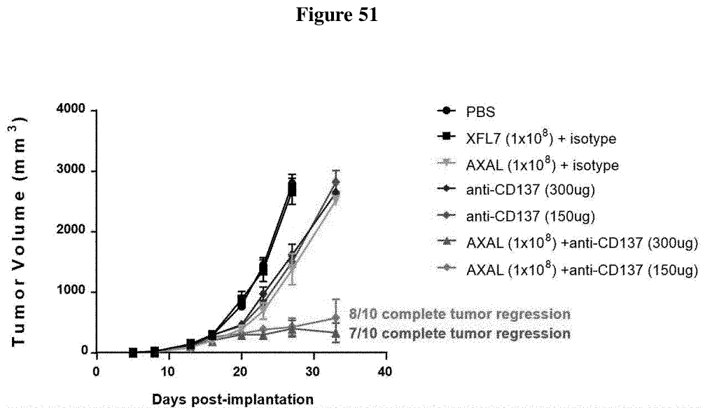

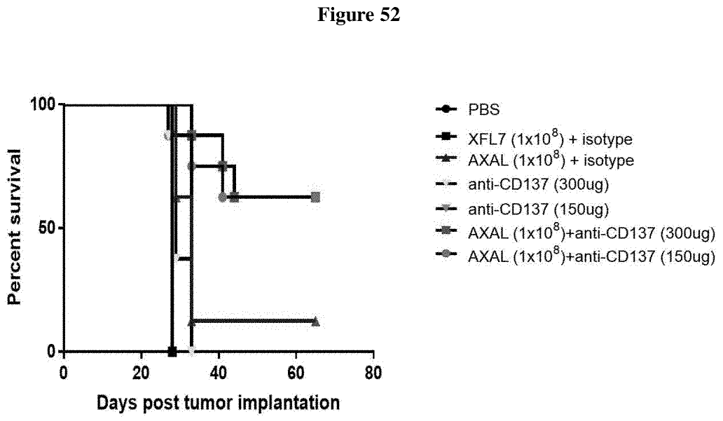

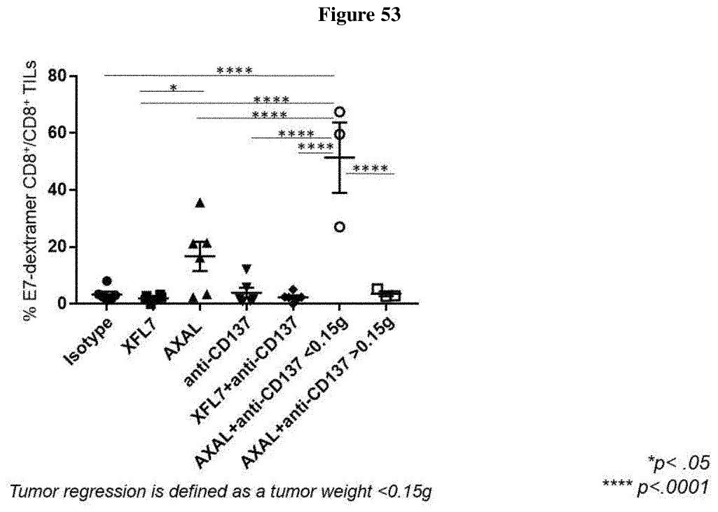

It was found that both dosages of anti-CD137 mAb synergize with AXAL to inhibit tumor growth (FIG. 51) and increase animal survival (FIG. 52). Complete tumor regression was observed in 80% of mice receiving AXAL+150 μg anti-CD137 mAb and in 70% of mice receiving AXAL+300 μg anti-CD137 mAb. Additionally, it was found that tumor regression induced by AXAL and anti-CD137 mAb is associated with increased levels of tumor-infiltrating HPV-E7-specific CD8+ T cells (FIG. 53). Therefore, Lm-based vectors combined with an agonistic anti-CD137 mAb synergize to provide effective antitumor immunity in the mouse TC-1 tumor tumor model.

### Example 17: Effects of Combining AXAL with Anti-CD137 mAb on the Generation of Durable Antitumor T Cell Responses

To evaluate the durability of antitumor T cell responses, mice with complete tumor regression after anti-CD137 mAb+AXAL treatment were re-challenged in the opposite flank with TC-1 tumor cells that were tested to be negative for mycoplasma 16 weeks post-primary tumor implantation. Of the four mice re-challenged, all remained tumor-free in both flanks for at least 5 additional weeks. The presence of HPV-E7-specific CD8+ T Cells in blood of re-challenged mice was analyzed pre- and post-re-challenge and results are shown in Table 11 below. Together, these findings show that treatment with anti-CD137 mAb after immunization with AXAL generates durable antitumor T cell responses that are effective at preventing tumor recurrence.

TABLE 11
Percentage of HPV-E7-specific T cells pre- and post-tumor re-challenge

| | % HPV-E7-specific/CD8+ | % HPV-E7-specific/CD8+ |
|---|---|---|
| Mouse ID | 1-day pre-challenge | 7-days post-challenge |
| Combo #1 | 3.4 | 1.9 |
| Combo #4 | 7.9 | 10.3 |
| Combo #7 | 2.4 | 7.1 |
| Combo #10 | 5.9 | 5.6 |
| Naive | 0.04 | 0.3 |

### Example 18: Effects of Combining AXAL with Anti-CD137 and Anti-CTLA-4/Anti-PD-1 mAbs on Tumor Growth and Animal Survival

To evaluate whether adding a checkpoint inhibitor (i.e., anti-CTLA-4/anti-PD-1 mAbs) to the AXAL+anti-CD137 mAb combination would enhance immune response, on Day 0 mice were injected with 1×10⁵ TC-1 tumor cells that were tested to be negative for mycoplasma. Starting on Day 11, the mice were administered Lm-treatment and mAb treatment according to the administration schedule in FIG. 49.

The groups tested in the experiment were as follows:

XLF7 (1E8)+isotypes

AXAL (1E8)+isotypes

Anti-CD 137 (150 μg) (LOB 12.3 clone)

Anti-PD-1 (100 μg) (RMP1-14 clone)

Anti-CTLA-4 (50 μg) (9H10 clone)

Anti-CD137+anti-PD-1

Anti-CD137+anti-CTLA-4

AXAL+anti-CD137

AXAL+anti-PD-1

AXAL+anti-CTLA-4

AXAL+anti-CD137+anti-PD-1

AXAL+anti-CD137+anti-CTLA-4

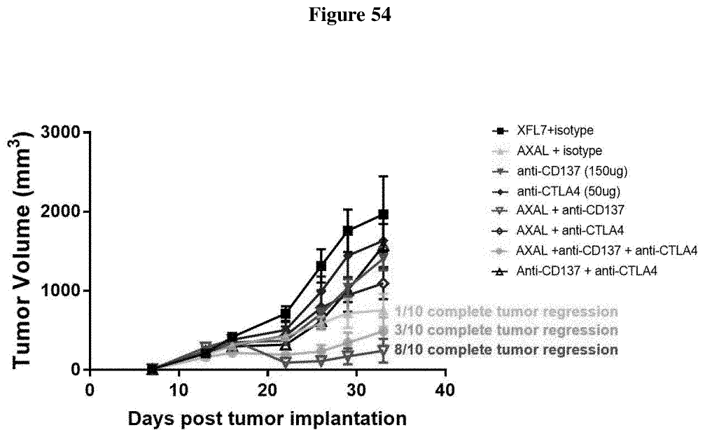

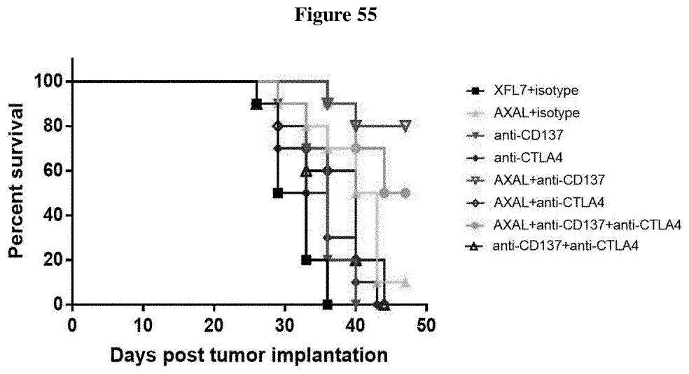

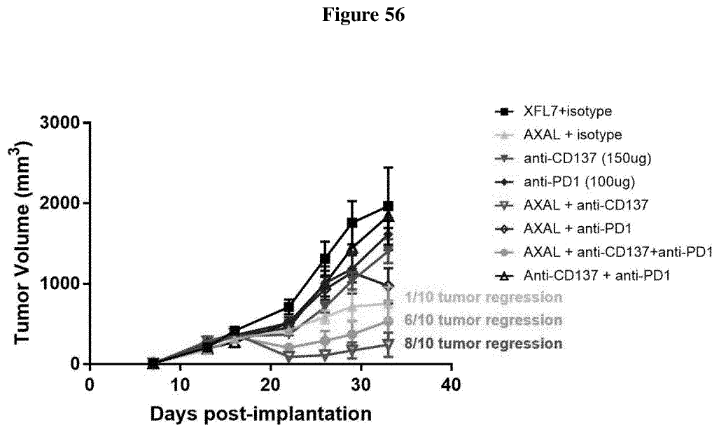

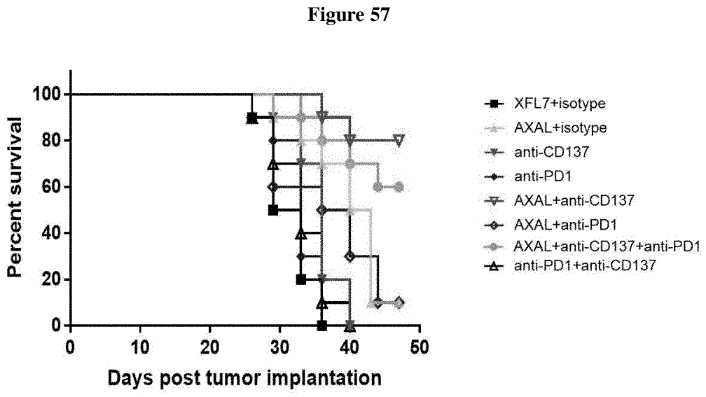

It was found that that the AXAL+anti-CD137 mAb+anti-CTLA-4 mAb triple combo is not more effective at tumor growth inhibition than AXAL+anti-CD137 mAb (FIG. 54) or increasing animal survival (FIG. 55). Likewise, it was found that that the AXAL+anti-CD137 mAb+anti-PD-1 mAb triple combo is not more effective at tumor growth inhibition than AXAL+anti-CD137 mAb (FIG. 56) or increasing animal survival (FIG. 57).

Therefore, adding a checkpoint inhibitor to the AXAL+anti-CD137 mAb combination does not enhance the ability of AXAL+anti-CD137 mAb to inhibit tumor growth effectively.

### Example 19: Anti-CD137 Synergizes with a Lm-Based Vector in a Murine Colorectal Cancer Model

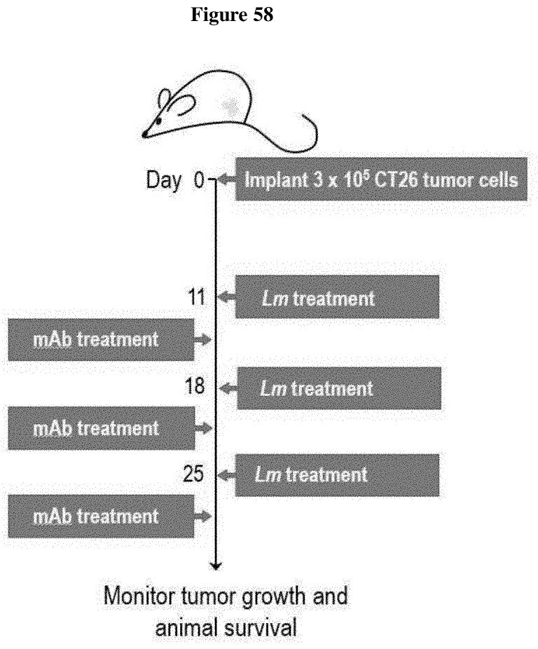

The synergy between anti-CD137 and a Lm-based vector was further tested in a second murine tumor model. The CT26 Tumor Model is a murine colorectal cancer model. On Day 0, mice were implanted with 3×10³ CT26 tumor cells. Starting on Day 11, the mice were administered Lm-treatment and mAb treatment according to the administration schedule in FIG. 58.

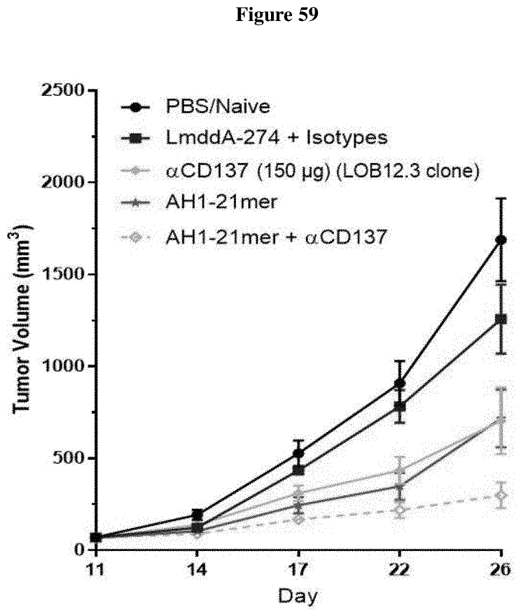

An enhanced reduction in tumor size was found in the combination therapy group (FIG. 59). Therefore, Lm-based vectors combined with an agonistic anti-CD137 mAb synergize to provide effective antitumor immunity in the mouse CT26 tumor models.

Having described embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed:

1. A method of promoting an antigen-specific memory T-cell population comprising, administering to a subject an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CD137 antibody or a functional fragment thereof.

2. A method for preventing reoccurrence of a tumor in a subject in need thereof, the method comprising administering to the subject an effective amount of a combination comprising, i. a immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CD137 antibody or a functional fragment thereof.

3. A method for generating a durable antitumor T cell response in a subject in need thereof, the method comprising administering to the subject an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CD137 antibody or a functional fragment thereof.

4. A method for preventing metastasis in a cancer patient at risk for metastasis, the method comprising administering to the patient an effective amount of a combination comprising, i. an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof, and ii. an effective amount of a composition comprising an anti-CD137 antibody or a functional fragment thereof.

5. The method of any one of claims 1-4, wherein a first dose of the composition comprising an anti-CD137 antibody or a functional fragment thereof is administered about 96 hours after the administration of a first of the immunogenic composition comprising a recombinant Listeria strain.

6. The method of any one of claims 1-4, wherein a first dose of the composition comprising an anti-CD137 antibody or a functional fragment thereof is administered about 72 hours after the administration of a first of the immunogenic composition comprising a recombinant Listeria strain.

7. The method of any one of claims 1-4, wherein a first dose of the composition comprising an anti-CD137 antibody or a functional fragment thereof is administered about 48 hours after the administration of a first dose the immunogenic composition comprising a recombinant Listeria strain.

8. The method of any one of claims 1-7, wherein the immunogenic composition comprising a recombinant Listeria strain is administered at a dose of about 1×10⁹ CFU.

9. The method of any one of claims 1-8, wherein the composition comprising an anti-CD137 antibody or a functional fragment thereof is administered at a dose between about 0.1 mg/kg and about 5 mg/kg.

10. The method of any one of claims 1-9, wherein the subject has a progression free survival of at least 3 months.

11. The method of any one of claims 1-10, wherein the heterologous antigen is a tumor-associated antigen.

12. The method of claim 11, wherein the tumor-associated antigen is a human papilloma virus (HPV) E7 antigen.

13. The method of claim 12, wherein the tumor-associated antigen is a HPV-16 E7 antigen.

14. The method of any one of claims 1-13, wherein the truncated LLO protein comprises SEQ ID NO: 2.

15. The method of any one of claims 1-14, wherein the recombinant Listeria strain is a recombinant Listeria monocytogenes strain.

16. The method of any one of claims 1-15, wherein the nucleic acid is in an extrachromosomal plasmid in the recombinant Listeria strain.

17. The method of claim 16, wherein the plasmid is stably maintained in the recombinant Listeria strain.

18. The method of any one of claims 1-17, wherein the Listeria strain comprises a mutation, deletion or inactivation in the endogenous prfA gene.

19. The method claim 18, wherein the prfA gene encodes a PrfA protein comprising a D133V mutation.

20. The method of any one of claims 1-19, wherein the nucleic acid further comprises a second open reading frame encoding a metabolic enzyme

21. The method of claim 20, wherein the metabolic enzyme complements the mutation, deletion or inactivation.

22. A kit, comprising a first container and a second container, wherein the first container comprises at least one dose of a composition comprising an anti-CD137 antibody or a functional fragment thereof, wherein the second container comprises at least one dose of an immunogenic composition comprising a recombinant Listeria strain comprising a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to a heterologous antigen or fragment thereof.

23. The kit of claim 22, wherein the composition comprising an anti-CD137 antibody or a functional fragment thereof is at a dose between about 0.1 mg/kg and about 5 mg/kg, and wherein the immunogenic composition comprising a recombinant Listeria strain is at a dose of about 1×10⁹ CFU.


SEQUENCE LISTING (42 sequences)

SEQ ID NO: 1 - protein, 32 aa, Listeria monocytogenes
KENSISSMAPPASPPASPKTPIEKKHADEIDK

SEQ ID NO: 2 - protein, 441 aa, Artificial Sequence
  N-terminal fragment of an LLO protein
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 3 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 4 - protein, 416 aa, Artificial Sequence
  LLO fragment
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 5 - protein, 390 aa, Artificial Sequence
  N-terminal fragment of an ActA protein
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 6 - DNA, 1170 bp, Artificial Sequence
  recombinant nucleotide encoding a fragment of an ActA protein
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 7 - protein, 14 aa, Artificial Sequence
  PEST amino acid sequence
KTEEQPSEVNTGPR

SEQ ID NO: 8 - protein, 28 aa, Artificial Sequence
  PEST amino acid sequence
KASVTDTSEGDLDSSMQSADESTPQPLK

SEQ ID NO: 9 - protein, 20 aa, Artificial Sequence
  PEST amino acid sequence
KNEEVNASDFPPPPTDEELR

SEQ ID NO: 10 - protein, 33 aa, Artificial Sequence
  PEST amino acid sequence
RGGIPTSEEFSSLNSGDFTDDENSETTEEEIDR

SEQ ID NO: 11 - protein, 17 aa, Streptococcus pyogenes
KQNTASTETTTTNEQPK

SEQ ID NO: 12 - protein, 17 aa, Streptococcus equisimilis
KQNTANTETTTTNEQPK

SEQ ID NO: 13 - protein, 98 aa, Artificial Sequence
  E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 14 - protein, 105 aa, Artificial Sequence
  E7 protein
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 15 - protein, 158 aa, Artificial Sequence
  E6 protein
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 16 - protein, 158 aa, Artificial Sequence
  E6 protein
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 17 - DNA, 22 bp, Artificial Sequence
  Primer
ggctcgagcatggagatacacc

SEQ ID NO: 18 - DNA, 28 bp, Artificial Sequence
  primer
ggggactagtttatggtttctgagaaca

SEQ ID NO: 19 - DNA, 31 bp, Artificial Sequence
  primer
gggggctagccctcctttgattagtatattc

SEQ ID NO: 20 - DNA, 28 bp, Artificial Sequence
  primer
ctccctcgagatcataatttacttcatc

SEQ ID NO: 21 - DNA, 27 bp, Artificial Sequence
  primer
cccgtcgaccagctcttcttggtgaag

SEQ ID NO: 22 - DNA, 25 bp, Artificial Sequence
  primer
gcggatcccatggagatacacctac

SEQ ID NO: 23 - DNA, 22 bp, Artificial Sequence
  primer
gctctagattatggtttctgag

SEQ ID NO: 24 - protein, 9 aa, Artificial Sequence
  H-2Db-restricted epitope from HPV E7
RAHYNIVTF

SEQ ID NO: 25 - protein, 9 aa, Artificial Sequence
  Epitope for HIV-GAG
AMQMLKETI

SEQ ID NO: 26 - protein, 9 aa, Artificial Sequence
  Epitope for Listeria LLO
GYKDGNEYI

SEQ ID NO: 27 - DNA, 55 bp, Artificial Sequence
  primer
gactacaaggacgatgaccgacaagtgataacccgggatctaaataaatccgttt

SEQ ID NO: 28 - DNA, 18 bp, Artificial Sequence
  primer
cctagctaaatttaatgt

SEQ ID NO: 29 - DNA, 18 bp, Artificial Sequence
  primer
cctagctaaatttaatga

SEQ ID NO: 30 - DNA, 21 bp, Artificial Sequence
  primer
taattttccccaagtagcagg

SEQ ID NO: 31-33 - skipped (3 entries)

SEQ ID NO: 34 - DNA, 6523 bp, Artificial Sequence
  plasmid pAdv142
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 35 - DNA, 36 bp, Artificial Sequence
  primer
cggaattcggatccgcgccaaatcattggttgattg

SEQ ID NO: 36 - DNA, 37 bp, Artificial Sequence
  primer
gcgagtcgacgtcggggttaatcgtaatgcaattggc

SEQ ID NO: 37 - DNA, 35 bp, Artificial Sequence
  primer
gcgagtcgacccatacgacgttaattcttgcaatg

SEQ ID NO: 38 - DNA, 39 bp, Artificial Sequence
  primer
gatactgcagggatccttcccttctcggtaatcagtcac

SEQ ID NO: 39 - DNA, 19 bp, Artificial Sequence
  primer
tgggatggccaagaaattc

SEQ ID NO: 40 - DNA, 22 bp, Artificial Sequence
  primer
ctaccatgtcttccgttgcttg

SEQ ID NO: 41 - DNA, 364 bp, Artificial Sequence
  Lovaxin_C_pGG55
ccaaaccctacaaaaacaagtttcatacagcctagctaaatttaatgttttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatcgtgattatctcaaaagatatgcccctaaattagatgaatggttttatttagcatgtcctgctacttggggaaaattaaattaa

SEQ ID NO: 42 - DNA, 364 bp, Listeria monocytogenes
ccaaaccctacaaaaacaagtttcatacagcctagctaaatttaatgatttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatcttgattatctcaaaagatatgcccctaaattagatgaatggttttatttagcatgtcctgctacttggggaaaattaaattaa